(19) United States
(12) Patent Application Publication
(10) Pub. No.: US 2016/0152995 A1
(43) Pub. Date: Jun. 2, 2016

(54) METHODS AND COMPOSITIONS TO IMPROVE THE SPREAD OF CHEMICAL SIGNALS IN PLANTS

(71) Applicants: PIONEER HI-BRED INTERNATIONAL, INC., Johnston, IA (US); E.I. DU PONT DE NEMOURS AND COMPANY, Wilmington, DE (US)

(72) Inventors: Kevin E. McBride, Davis, CA (US); Brian McGonigle, Wilmington, DE (US); Narendra S. Yadav, Wilmington, DE (US)

(21) Appl. No.: 14/775,531

(22) PCT Filed: Mar. 11, 2014
(86) PCT No.: PCT/US14/23451

§ 371 Date: Sep. 11, 2015

Related U.S. Application Data
(60) Provisional application No. 61/776,296, filed on Mar. 11, 2013.

(51) Int. Cl. C12N 15/82

(52) U.S. Cl. CPC C12N 15/8238; C12N 15/8218

(57) ABSTRACT

Compositions and methods are provided which employ a chemical-gene switch which may comprise at least three components. The first component comprises a polynucleotide encoding a chemically-regulated transcriptional repressor; the second component comprises a repressible promoter operably linked to a polynucleotide of interest, and the third component comprises a gene silencing construct operably linked to a second repressible promoter, wherein the gene silencing construct encodes a silencing element that decreases the level of mRNA encoding the chemically-regulated transcriptional repressor. Expression of the polynucleotide of interest and the silencing construct is controlled by providing the appropriate chemical ligand. Transient induction from the chemical ligand leads to the production of the silencing element, and the destruction of the mRNA encoding the chemically-regulated transcriptional repressor.

### REFERENCE TO SEQUENCE LISTING

The Sequence Listing submitted 11 Mar. 2014, as a text file named 36446_0068P1_Seq_List.txt, created on 5 Mar. 2014, and having a size of 2,258,550 bytes is hereby incorporated by reference pursuant to 37 C.F.R. §1.52(e)(5).

### FIELD OF THE INVENTION

The invention relates to the field of molecular biology, more particularly to the regulation of gene expression.

### BACKGROUND

The tetracycline operon system, comprising repressor and operator elements, was originally isolated from bacteria. The operon system is tightly controlled by the presence of tetracycline, and self-regulates the level of expression of tetA and tetR genes. The product of tetA removes tetracycline from the cell. The product of tetR is the repressor protein that binds to the operator elements with a K_{d} of about 10 pM in the absence of tetracycline, thereby blocking expression or tetA and tetR.

This system has been modified to control expression of other polynucleotides of interest, and/or for use in other organisms, mainly for use in animal systems. Tet operon based systems have had limited use in plants, at least partially due to problems with the inducers which are typically antibiotic compounds, and sensitive to light. Moreover, other chemical-gene switches employed in plants require the chemical ligand to contact and penetrate the cell for the switch to be activated. This limits the extent to which a chemical-gene switch can be activated in tissues or organisms not easily contacted with the chemical ligand.

There is a need to regulate expression of sequences of interest in organisms. Chemical-gene switch compositions and methods to regulate expression in response to compounds, such as sulfonylurea compounds, are provided.

### SUMMARY

Compositions and methods are provided which employ a chemical-gene switch. The chemical-gene switch disclosed herein comprises at least three components. The first component comprises a polynucleotide encoding a chemically-regulated transcriptional repressor; the second component comprises a repressible promoter operably linked to a polynucleotide of interest, and the third component comprises a gene silencing construct operably linked to a second repressible promoter, wherein the gene silencing construct encodes a silencing element that decreases the level of the chemically-regulated transcriptional repressor. Expression of the polynucleotide of interest and the silencing construct is controlled by providing the appropriate chemical ligand. Transient induction from the chemical ligand leads to the production of the silencing element, and the destruction of the mRNA encoding the chemically-regulated transcriptional repressor. The presence of the silencing element maintains a state of de-repression. Since, in some embodiments, the silencing elements are cell non-autonomous, the state of de-repression becomes more distributed throughout the plant beyond where the chemical ligand reaches.

### BRIEF DESCRIPTION OF DRAWINGS

FIG. 1 provides a non-limiting example of a sulfonylurea chemical-gene switch.

FIG. 2 provides a non-limiting example for modifying a sulfonylurea chemical-gene switch with siRNA.

FIG. 3 provides a non-limiting schematic for optimizing the dosage of repressor transcript for siRNA efficacy thru repressor auto-regulation.

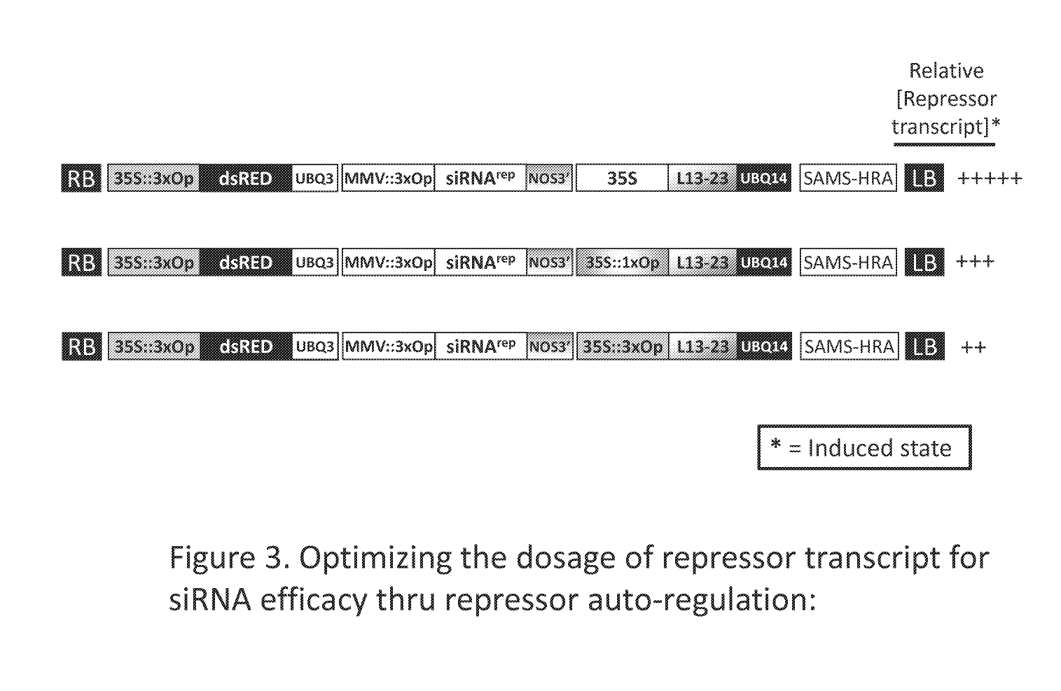

FIG. 4 provides a non-limiting example of targeting the repressor EsR (L13-23) transcript.

FIG. 5 demonstrates induction in test and control transgenic tobacco plants.

FIG. 6 shows extended and more thorough Ethametsulfuron induction in tobacco seedlings.

FIG. 7 demonstrates long term derepression in tobacco plants induced with Ethametsulfuron during germination.

FIG. 8 provides a summary of source diversity, library design, hit diversity, and population bias for several generations of sulfonylurea repressor shuffling libraries. A dash (“-”) indicates no amino acid diversity introduced at that position in that library. An X indicates that the library oligonucleotides were designed to introduce complete amino acid diversity (any of 20 amino acids) at that position in that library. Residues in bold indicate bias during selection with larger font size indicating a greater degree of bias in the selected population. Residues in parentheses indicate selected random mutations. The phylogenetic diversity pool was derived from a broad family of 34 tetracycline repressor sequences.

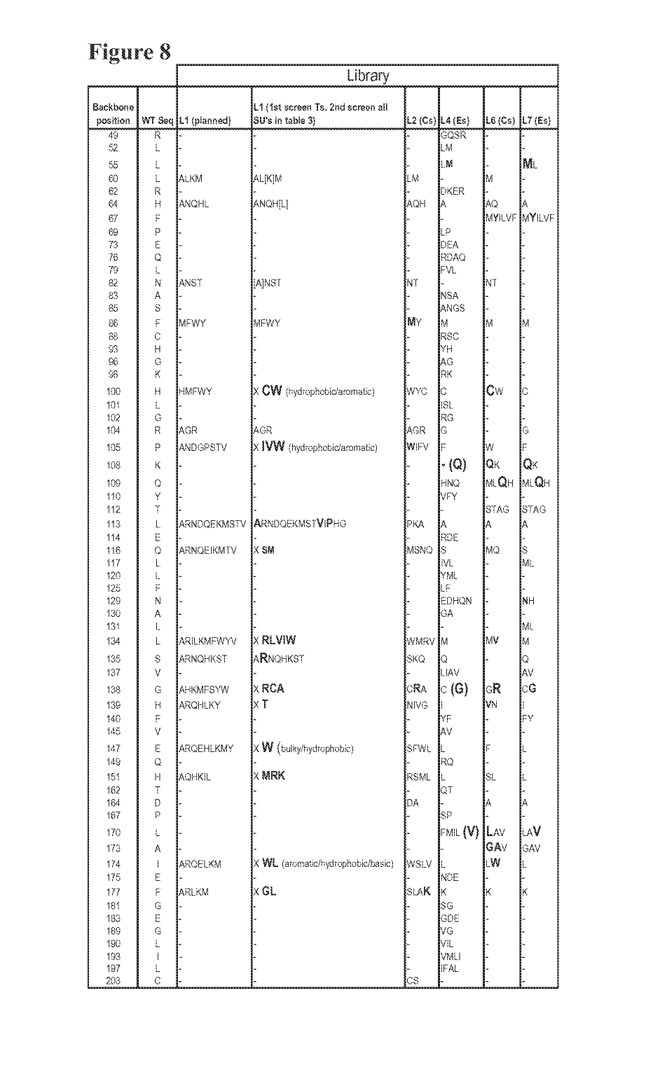

FIG. 9 provides a summary of source diversity, library design, hit diversity, and population bias for several generations of sulfonylurea repressor shuffling libraries Description of libraries L10, L11, L12, L13, L15 and resulting sequence incorporation biases. A dash (“-”) indicates no amino acid diversity introduced at that position in that library. An X indicates that the library oligonucleotides were designed to introduce complete amino acid diversity (any of 20 amino acids) at that position in that library. Residues in bold indicate bias during selection with larger font size indicating a greater degree of bias in the selected population. Residues in parentheses indicate selected mutations.

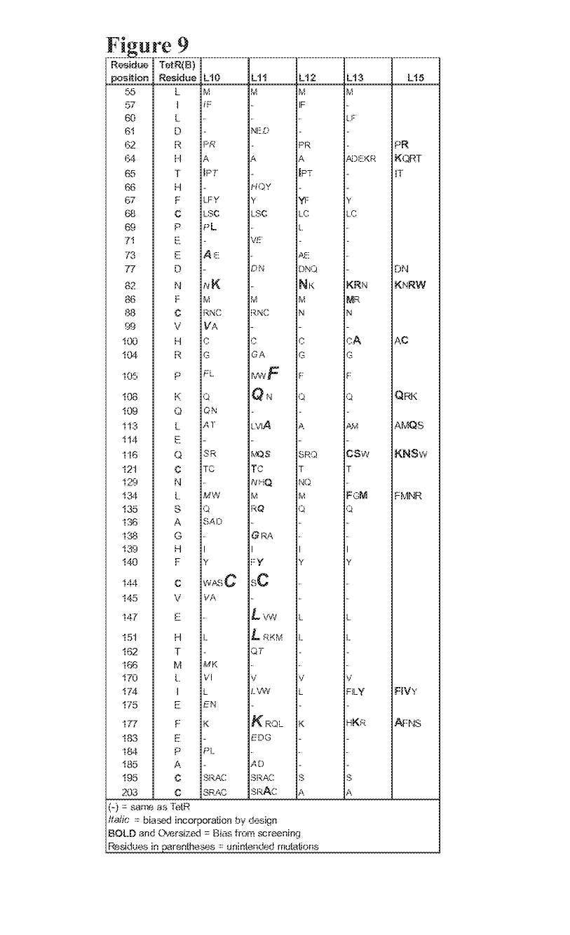

FIG. 10 provides B-galactosidase assays of hits from saturation mutagenesis at position D178.

FIG. 11 shows the proximity of residues L131 and T134 to the sulfonylurea differentiating side groups of Chlorsulfuron bound CsR(CsL4.2-20).

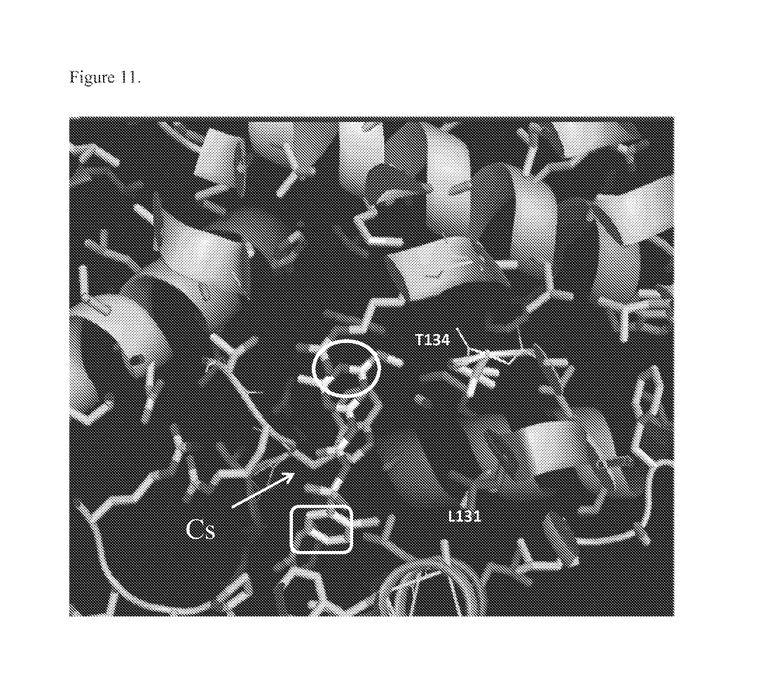

### DETAILED DESCRIPTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

One of the main limitations of any chemically inducible system in multicellular organisms is the penetration and even distribution of the inducer throughout all tissues (due to variable movement or metabolism). The result is the possibility of uneven (or lack of) targeted gene induction in the tissues or cell types of interest. To address this limitation, methods and compositions are provided which employ additional genetic factors to affect the spread of de-repression.

Specifically, the compositions and methods disclosed herein employ a chemical-gene switch. The chemical-gene switch, disclosed herein comprises at least three components. The first component comprises a polynucleotide encoding a chemically-regulated transcriptional repressor; the second component comprises a repressible promoter operably linked to a polynucleotide of interest, and the third component comprises a gene silencing construct operably linked to a second repressible promoter, wherein the gene silencing construct encodes a silencing element that decreases the level of the chemically-regulated transcriptional repressor. Expression of the polynucleotide of interest and the silencing construct is controlled by providing the appropriate chemical ligand. Transient induction from the chemical ligand leads to the production of the silencing element, and a decrease in the level of the chemically-regulated transcriptional repressor. The presence of the silencing element maintains a state of de-repression. Since, in some embodiments, the silencing elements are cell non-autonomous, the state of de-repression becomes distributed throughout the plant beyond where the chemical ligand physically reaches.

As explained in further detail herein, the activity of the chemical-gene switch can be controlled by selecting the combination of elements used in the switch. These include, but are not limited to, the type of promoter operably linked to the chemically-regulated transcriptional repressor, the chemically-regulated transcriptional repressor, the repressible promoter operably linked to the polynucleotide of interest, the polynucleotide of interest, the repressible promoter operably linked to the gene silencing construct, and the gene silencing construct. Further control is provided by selection, dosage, conditions, and/or timing of the application of the chemical ligand.

### II. Components of the Chemical-Gene Switch

The compositions and methods disclosed herein employ a chemical-gene switch comprising a polynucleotide of interest construct; a chemically-regulated transcriptional repressor construct; and a gene silencing construct encoding a silencing element that decreases the level of the chemically-regulated transcriptional repressor. Each of these components is discussed in more detail below.

1. Polynucleotide Encoding a Chemically-Regulated Transcriptional Repressor

a. Chemically-Regulated Transcriptional Repressor

As used herein, a “chemically-regulated transcriptional repressor” comprises a polypeptide that contains a DNA binding domain and a ligand binding domain. In the absence of the chemical ligand, the chemically-regulated transcriptional repressor binds an operator of a promoter and represses the activity of the promoter and thereby represses expression of the polynucleotide operably linked to said promoter. In the presence of an effective concentration of the chemical ligand, the chemically-regulated transcriptional repressor will bind the chemical ligand. The ligand-bound chemically-regulated transcriptional repressor can no longer repress transcription from the promoter containing the operator. Variants and fragments of a chemically-regulated transcriptional repressor will retain this activity.

By “repress transcription” is intended to mean a reduction or an elimination of transcription of a given polynucleotide. Repression of transcription can therefore comprise the complete elimination of transcription from a given promoter or it can comprise a reduction in the amount of transcription from the promoter when compared to the level of transcription occurring from an appropriate control in the absence of the chemical ligand. A reduction can comprise any statistically significant decrease including, a decrease of at least 1%, 5%, 10%, 20%, 30%, 40%, 50%, 60%, 70%, 80%, 90%, 100%, 150%, 200% or greater or at least a 1, 2, 3, 4, 5, 6, 7, 8, 9, or 10 fold decrease.

A variety of chemically-regulated transcriptional repressors can be employed in the methods and compositions disclosed herein. In one embodiment, the chemically-regulated transcriptional repressor is a tetracycline transcriptional repressor (TetR), whose binding to an operator is influenced by tetracycline or a derivative thereof. In one embodiment, the chemically-regulated transcription repressor is from the tetracycline class A, B, C, D, E, G, H, J and Z of repressors. An example of the TetR(A) class is found on the Tn1721 transposon and deposited under GenBank accession X61307, cross-referenced under gi48198, with encoded protein accession CAA43639, cross-referenced under gi48195 and UniProt accession Q56321. An example of the TetR(B) class is found on the Tn10 transposon and deposited under GenBank accession X00694, cross-referenced under gi43052, with encoded protein accession CAA25291, cross-referenced under gi43052 and UniProt accession PO4483. An example of the TetR(C) class is found on the pSC101 plasmid and deposited under GenBank Accession M36272, cross-referenced under gi150945, with encoded protein accession AAA25677, cross-referenced under gi150946. An example of the TetR(D) class is found in Salmonella ordonez and deposited under GenBank Accession X65876, cross-referenced under gi49073, with encoded protein accession CAA46707, cross-referenced under gi49075 and UniProt accessions POACT5 and P09164. An example of the TetR(E) class was isolated from E. coli transposon Tn10 and deposited under GenBank Accession M34933, cross-referenced under gi155019, with encoded protein accession AAA98409, cross-referenced under gi155020. An example of the TetR(G) class was isolated from Vibrio anguillarium and deposited under GenBank Accession S52438, cross-referenced under gi262928, with encoded protein accession AAB24797, cross-referenced under gi262929. An example of the TetR(H) class is found on plasmid pMV111 isolated from Pasteurella multocida and deposited under GenBank Accession U00792, cross-referenced under gi392871, with encoded protein accession AAC43249, cross-referenced under gi392872. An example of the TetR(J) class was isolated from Proteus mirabilis and deposited under GenBank Accession AF038993, cross-referenced under gi4104704, with encoded protein accession AAD12754, cross-referenced under gi4104706. An example of the TetR(Z) class was found on plasmid pAGI isolated from Corynebacterium glutamicum and deposited under GenBank Accession AF121000, cross-referenced under gi4583389, with encoded protein accession AAD25064, cross-referenced under gi4583390. In other examples the wild type tetracycline repressor is a class B tetracycline repressor protein, or the wild type tetracycline repressor is a class D tetracycline repressor protein. The properties, domains, motifs and function of tetracycline transcriptional repressors are well known, as are standard techniques and assays to evaluate any derived repressor comprising one or more amino acid substitutions.

Numerous variants of TetR have been identified and/or derived and extensively studied. In the context of the tetracycline transcriptional repressor system, the effects of various mutations, modifications and/or combinations thereof have been used to extensively characterize and/or modify the properties of tetracycline repressors, such as cofactor binding, ligand binding constants, kinetics and dissociation constants, operator binding sequence constraints, cooperativity, binding constants, kinetics and dissociation constants and fusion protein activities and properties. Variants include TetR variants with a reverse phenotype of binding the operator sequence in the presence of tetracycline or an analog thereof, variants having altered operator binding properties, variants having altered operator sequence specificity and variants having altered ligand specificity and fusion proteins. See, for example, Isackson & Bertrand (1985) PNAS 82:6226-6230; Smith & Bertrand (1988) J Mol Biol 203:949-959; Altschmied et al. (1988) EMBO J7:4011-4017; Wissmann et al. (1991) EMBO J 10:4145-4152; Baumeister et al. (1992) J Mol Biol 226:1257-1270; Baumeister et al. (1992) Proteins 14:168-177; Gossen & Bujard (1992) PNAS 89:5547-5551; Wasylewski et al. (1996) J Protein Chem 15:45-58; Berens et al. (1997) J Biol Chem 272:6936-6942; Baron et al. (1997) Nucl Acids Res 25:2723-2729; Helbl & Hillen (1998) J Mol Biol 276:313-318; Urlinger et al. (2000) PNAS 97:7963-7968; Kamionka et al. (2004) Nucl Acids Res 32:842-847; Bertram et al. (2004) J Mol Microbiol Biotechnol 8:104-110; Scholz et al. (2003) J Mol Biol 329: 217-227; and US2003/0186281, each of which is herein incorporated by reference in its entirety.

The modular architecture of chemically-regulated transcriptional repressor proteins and the commonality of helix-turn-helix DNA binding domains allows for the creation of sulfonylurea-responsive repressor polypeptides. Thus, in some embodiments, the chemically-regulated transcription repressor comprises a sulfonylurea-responsive transcriptional repressor (SuR) polypeptide. As used herein, a “sulfonylurea-responsive transcriptional repressor” or “SuR” comprises any chemically-regulated transcriptional repressor polypeptide whose binding to an operator sequence is controlled by a ligand comprising a sulfonylurea compound or a derivative thereof. In the absence of the sulfonylurea chemical ligand, the SuR binds a given operator of a promoter and represses the activity of the promoter and thereby represses expression of the polynucleotide operably linked to said promoter. Upon interaction of the SuR with its chemical ligand, the SuR is no longer able to repress transcription of the promoter containing the operator.

The SuR can be designed to contain a variety of different DNA binding domains and thereby bind a variety of different operators and influence transcription. In one embodiment, the SuR polypeptide comprises a DNA binding domain that specifically binds to a tetracycline operator. Thus, in specific embodiments, the SuR polypeptide or the polynucleotide encoding the same can comprise a DNA binding domain, including but not limited to, an operator DNA binding domain from repressors included tet, lac, trp, phd, arg, LexA, phiCh1 repressor, lambda C1 and Cro repressors, phage X repressor, MetJ, phir1t rro, phi434 C1 and Cro repressors, RafR, gal, ebg, uxuR, exuR, ROS, SinR, PurR, FruR, P22 C2, TetC, AcrR, Betl, Bm3R1, EnvR, QacR, MtrR, TcmR, Ttk, YbiH, YhgD, and mu Ner, or DNA binding domains in Interpro families including, but not limited to, IPR001647, IPR010982, and IPR01199, or an active variant or fragment thereof. Thus, the DNA binding specificity can be altered by fusing a SuR ligand binding domain to an alternate DNA binding domain. For example, the DNA binding domain from TetR class D can be fused to a SuR ligand binding domain to create SuR polypeptides that specifically bind to polynucleotides comprising a class D tetracycline operator. In some examples, a DNA binding domain variant or derivative can be used. For example, a DNA binding domain from a TetR variant that specifically recognizes a tetO-4C operator or a tetO-6C operator could be used (Helbl & Hillen (1998) J Mol Biol 276:313-318; Helbl et al. (1998) J Mol Biol 276:319-324).

In some examples, the chemically-regulated transcriptional repressor, or the polynucleotide encoding the same, includes a SuR polypeptide comprising a ligand binding domain comprising at least one amino acid substitution to a wild type tetracycline repressor protein ligand binding domain fused to a heterologous operator DNA binding domain which specifically binds to a polynucleotide comprising the operator sequence or derivative thereof, wherein repressor-operator binding is regulated by the absence or presence of a sulfonylurea compound. In specific embodiments, the heterologous operator DNA binding domain comprises a tetracycline operator sequence or active variant or fragment thereof, such that the repressor-operator binding is regulated by the absence or presence of a sulfonylurea compound. Non-limiting examples of SuR polypeptides are set forth in U.S. Utility application Ser. No. 13/086,765, filed on Apr. 14, 2011 and in US Application Publication 2010-0105141, both of which are herein incorporated by reference in their entirety.

In some examples, the SuR polypeptides, or polynucleotide encoding the same, comprise an amino acid substitution in the ligand binding domain of a wild type tetracycline repressor protein. In class B and D wild type TetR proteins, amino acid residues 6-52 represent the DNA binding domain. The remainder of the protein is involved in dimerization, ligand binding and subsequent allosteric modification. For class B TetR residues 53-207 represent the ligand binding domain, while residues 53-218 comprise the ligand binding domain for the class D TetR. In some embodiments, the SuR polypeptides comprise at least one amino acid substitution in the ligand binding domain of a wild type TetR(B) protein.

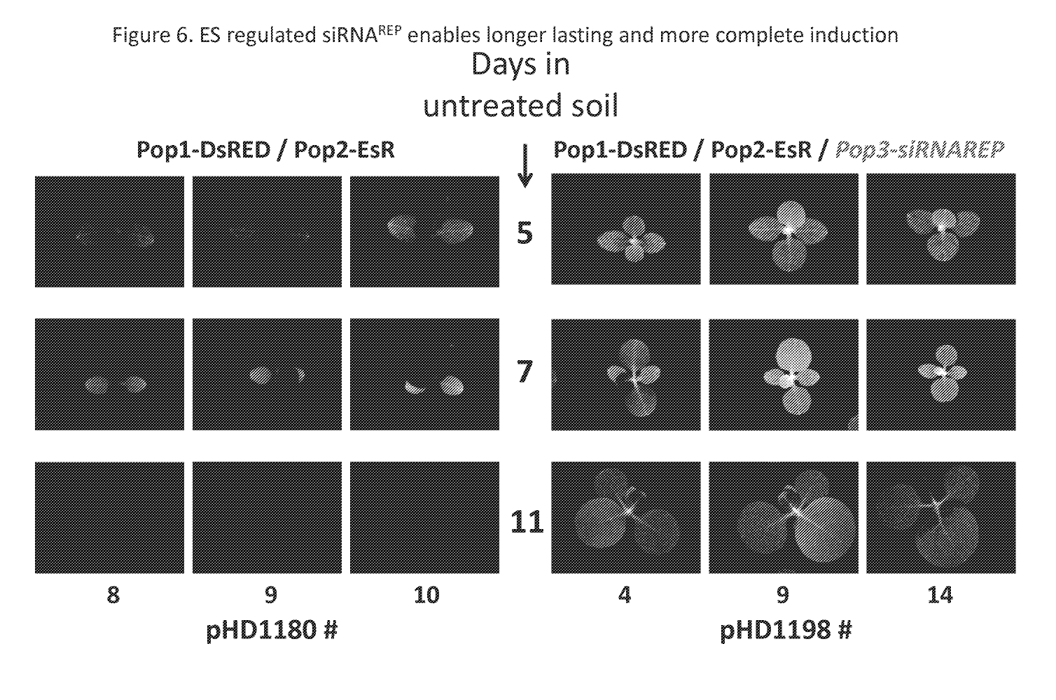

In some examples, the SuR polypeptide, or polynucleotide encoding the same, comprise an amino acid, or any combination of amino acids, corresponding to equivalent amino acid positions selected from the amino acid diversity shown in FIG. 6, wherein the amino acid residue position shown in FIG. 6 corresponds to the amino acid numbering of a wild type TetR(B). In some examples, the SuR polypeptides (or the polynucleotides encoding the same) comprise a ligand binding domain comprising at least 10%, 20%, 30%, 40%, 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid residues shown in FIG. 6, wherein the amino acid residue position corresponds to the equivalent position using the amino acid numbering of a wild type TetR(B). In some examples, the wild type TetR(B) is SEQ ID NO:1.

In other examples, the SuR polypeptide, or polynucleotide encoding the same, comprises a ligand binding domain comprising at least one amino acid substitution at a residue position selected from the group consisting of position 55, 60, 64, 67, 82, 86, 100, 104, 105, 108, 113, 116, 134, 135, 138, 139, 147, 151, 170, 173, 174, 177 and any combination thereof, wherein the amino acid residue position and substitution corresponds to the equivalent position using the amino acid numbering of a wild type TetR(B). In some examples, the SuR polypeptide further comprises at least one amino acid substitution at an amino acid residue position selected from the group consisting of 109, 112, 117, 131, 137, 140, 164 and any combination thereof. In some examples, the wild type TetR(B) is SEQ ID NO:1.

In other embodiments, the SuR polypeptide, or polynucleotide encoding the same, comprises at least about 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the ligand binding domain of a wild type TetR(B) exemplified by amino acid residues 53-207 of SEQ ID NO:1, wherein the sequence identity is determined over the full length of the ligand binding domain using a global alignment method. In some examples the global alignment method uses the GAP algorithm with default parameters for an amino acid sequence % identity and % similarity using GAP Weight of 8 and Length Weight of 2, and the BLOSUM62 scoring matrix.

In other examples, the SuR polypeptide, or polynucleotide encoding the same, comprises at least about 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to a wild type TetR(B) exemplified by SEQ ID NO:1, wherein the sequence identity is determined over the full length of the polypeptide using a global alignment method. In some examples the global alignment method uses the GAP algorithm with default parameters for an amino acid sequence % identity and % similarity using GAP Weight of 8 and Length Weight of 2, and the BLOSUM62 scoring matrix.

Additional SuR polypeptides, or polynucleotide encoding the same, comprising at least about 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the ligand binding domain of a SuR polypeptide selected from the group consisting of SEQ ID NO:3-419, 863-870, 884-889, 1381-1568 and/or 2030-2110, wherein the sequence identity is determined over the full length of the ligand binding domain using a global alignment method. The ligand binding domain of SEQ ID NO: 3-419, 863-870, 884-889, 1381-1568 and/or 2030-2110 comprises amino acids 53-207. In some examples the global alignment method uses the GAP algorithm with default parameters for an amino acid sequence % identity and % similarity using GAP Weight of 8 and Length Weight of 2, and the BLOSUM62 scoring matrix.

In other examples, the SuR polypeptide, or polynucleotide encoding the same, have at least about 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to a SuR polypeptide selected from the group consisting of SEQ ID NO:3-419, 863-870, 884-889, 1381-1568 and/or 2030-2110, wherein the sequence identity is determined over the full length of the polypeptide using a global alignment method. In some examples the global alignment method uses the GAP algorithm with default parameters for an amino acid sequence % identity and % similarity using GAP Weight of 8 and Length Weight of 2, and the BLOSUM62 scoring matrix.

Non-limiting examples of SuR polypeptides, or polynucleotide encoding the same, comprise an amino acid sequence that can be optimally aligned with a polypeptide sequence of L7-1A04 (SEQ ID NO:220), L1-22 (SEQ ID NO:7), L1-29 (SEQ ID NO:10), L1-02 (SEQ ID NO:3), L1-07 (SEQ ID NO:4), L1-20 (SEQ ID NO:6), L1-44 (SEQ ID NO:13), L6-3A09 (SEQ ID NO:402), L6-3H02 (SEQ ID NO:94), L7-4E03 (SEQ ID NO:403), L10-84(B12) (SEQ ID NO:404), or L13-46 (SEQ ID NO:405) to generate a percent sequence identity of at least 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity, wherein the sequence identity is determined by BLAST alignment using the BLOSUM62 matrix, a gap existence penalty of 11, and a gap extension penalty of 1. In some examples, the SuR polypeptides, or polynucleotide encoding the same, comprise an amino acid sequence that can be optimally aligned with a polypeptide sequence of L7-1A04 (SEQ ID NO:220) to generate a percent sequence identity of at least 88% sequence identity, optimally aligned with a polypeptide sequence of L1-22 (SEQ ID NO:7) to generate a percent sequence identity of at least 92% sequence identity, optimally aligned with a polypeptide sequence of L1-07 (SEQ ID NO:4) to generate a percent sequence identity of at least 93% sequence identity, optimally aligned with a polypeptide sequence of L1-20 (SEQ ID NO:6) to generate a percent sequence identity of at least 93% sequence identity, optimally aligned with a polypeptide sequence of L1-44 (SEQ ID NO:13) to generate a percent sequence identity of at least 93% sequence identity, optimally aligned with a polypeptide sequence of L6-3H02 (SEQ ID NO:94) to generate a percent sequence identity of at least 90% sequence identity, optimally aligned with a polypeptide sequence of L10-84(B12) (SEQ ID NO:404) to generate a percent sequence identity of at least 86% sequence identity, or optimally aligned with a polypeptide sequence of L13-46 (SEQ ID NO:405) to generate a percent sequence identity of at least 86% sequence identity, wherein the sequence identity is determined by BLAST alignment using the BLOSUM62 matrix, a gap existence penalty of 11, and a gap extension penalty of 1. In some examples the percent identity is determined using a global alignment method using the GAP algorithm with default parameters for an amino acid sequence % identity and % similarity using GAP Weight of 8 and Length Weight of 2, and the BLOSUM62 scoring matrix.

In further embodiments, the SuR polypeptides, or polynucleotide encoding the same, comprise an amino acid sequence that can be optimally aligned with a polypeptide sequence of L7-1A04 (SEQ ID NO:220), L1-22 (SEQ ID NO:7), L1-29 (SEQ ID NO:10), L1-02 (SEQ ID NO:3), L1-07 (SEQ ID NO:4), L1-20 (SEQ ID NO:6), L1-44 (SEQ ID NO:13), L6-3A09 (SEQ ID NO:402), L6-3H02 (SEQ ID NO:94), L7-4E03 (SEQ ID NO:403), L10-84(B12) (SEQ ID NO:404), or L13-46 (SEQ ID NO:405) to generate a BLAST similarity score of at least 400, 425, 450, 475, 500, 525, 550, 575, 600, 625, 650, 675, 700, 750, 800, 850, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, or 1200 wherein the BLAST alignment used the BLOSUM62 matrix, a gap existence penalty of 11, and a gap extension penalty of 1.

In some examples, the SuR polypeptides, or polynucleotide encoding the same, comprise an amino acid sequence that can be optimally aligned with a polypeptide sequence of L1-29 (SEQ ID NO:10) to generate a BLAST similarity score of at least 1006, optimally aligned with a polypeptide sequence of L1-07 (SEQ ID NO:4) to generate a BLAST similarity score of at least 996, optimally aligned with a polypeptide sequence of L6-3A09 (SEQ ID NO:402) to generate a BLAST similarity score of at least 978, optimally aligned with a polypeptide sequence of L7-4E03 (SEQ ID NO:403) to generate a BLAST similarity score of at least 945, or optimally aligned with a polypeptide sequence of L13-46 (SEQ ID NO:405) to generate a BLAST similarity score of at least 819, wherein the BLAST alignment used the BLOSUM62 matrix, a gap existence penalty of 11, and a gap extension penalty of 1.

In some examples, the SuR polypeptides, or polynucleotide encoding the same, comprise a ligand binding domain from a polypeptide selected from the group consisting of SEQ ID NO:3-419, 863-870, 884-889, 1381-1568 and/or 2030-2110. In some examples, the SuR polypeptides, or polynucleotide encoding the same, comprise an amino acid sequence selected from the group consisting of SEQ ID NO:3-419. In some examples the isolated SuR polypeptide is selected from the group consisting of SEQ ID NO:3-419, 863-870, 884-889, 1381-1568 and/or 2030-2110, and the sulfonylurea compound is selected from the group consisting of a chlorsulfuron, an ethametsulfuron, a metsulfuron, a sulfometuron, a tribenuron, a chlorimuron, a nicosulfuron, a rimsulfuron and a thifensulfuron.

In non-limiting embodiments, the SuR polypeptides can have an equilibrium binding constant for a sulfonylurea compound greater than 0.1 nM and less than 10 μM. In some examples, the SuR polypeptide has an equilibrium binding constant for a sulfonylurea compound of at least 0.1 nM, 0.5 nM, 1 nM, 10 nM, 50 nM, 100 nM, 250 nM, 500 nM, 750 nM, 1 μM, 5 μM, 7 μM but less than 10 μM. In other examples, the SuR polypeptide has an equilibrium binding constant for a sulfonylurea compound of at least 0.1 nM, 0.5 nM, 1 nM, 10 nM, 50 nM, 100 nM, 250 nM, 500 nM, 750 nM but less than 1 μM. In some embodiments, the SuR polypeptide has an equilibrium binding constant for a sulfonylurea compound greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples, the sulfonylurea compound is a chlorsulfuron, an ethametsulfuron, a metsulfuron, a sulfometuron, a tribenuron, a chlorimuron, a nicosulfuron, a rimsulfuron and/or a thifensulfuron.

In some examples, the SuR polypeptides have an equilibrium binding constant for an operator sequence greater than 0.1 nM and less than 10 μM. In some examples the SuR polypeptide has an equilibrium binding constant for an operator sequence of at least 0.1 nM, 0.5 nM, 1 nM, 10 nM, 50 nM, 100 nM, 250 nM, 500 nM, 750 nM, 1 μM, 5 μM, 7 μM but less than 10 μM. In some examples, the SuR polypeptide has an equilibrium binding constant for an operator sequence of at least 0.1 nM, 0.5 nM, 1 nM, 10 nM, 50 nM, 100 nM, 250 nM, 500 nM, 750 nM but less than 1 μM. In some examples the SuR polypeptide has an equilibrium binding constant for an operator sequence greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples, the operator sequence is a Tet operator sequence. In some examples, the Tet operator sequence is a TetR(A) operator sequence, a TetR(B) operator sequence, a TetR(D) operator sequence, TetR(E) operator sequence, a TetR(H) operator sequence, or a functional derivative thereof.

Various chemical ligands, including exemplary sulfonylurea chemical ligands, and the level and manner of application are discussed in detail elsewhere herein.

b. Promoters for Expression of the Chemically-Regulated Transcriptional Repressor

The polynucleotide encoding the chemically-regulated transcriptional repressor is operably linked to a promoter that is active in a plant. Various promoters can be employed and non-limiting examples are set forth elsewhere herein. Briefly, the polynucleotide encoding the chemically-regulated transcriptional repressor can be operably linked to constitutive promoter, an inducible promoter, or tissue-preferred promoter. In specific embodiments, the chemically-regulated transcriptional repressor is operably linked to a non-constitutive promoter, including but not limited to a tissue-preferred promoter, an inducible promoter, a repressible promoter, a developmental stage preferred promoter, or a promoter having more than one of these properties. In some examples expression of the polynucleotide of interest is primarily regulated in roots, leaves, stems, flowers, silks, anthers, pollen, meristem, germline, seed, endosperm, embryos, or progeny.

In other embodiments, the chemically-regulated transcriptional repressor can be operably linked to a repressible promoter, thus allowing the chemically-regulated transcriptional repressor to auto-regulate its own expression. It has been mathematically predicted that negative auto-regulation would not only dampen fluctuations in gene expression but also enhance signal response time in regulatory circuits involving repressor molecules (Savageau (1974) Nature 252:542-549). This principle was demonstrated in E. coli using synthetic gene circuitry (Rosenfeld et al. (2002) J Mol Biol 323:785-793) and in yeast (Nevozhay (2009) Proc Natl Acad Sci USA 106:5123-5128). Thus, in specific embodiments, the polynucleotide encoding the chemically-regulated transcriptional repressor can be operably linked to a repressible promoter comprising at least one, two, three or more operators (including a tet operator, such as that set forth in SEQ ID NO:848 or an active variant or fragment thereof) regulating expression of said repressor. Non-limiting repressible promoters for expression of the chemically-regulated transcriptional repressor, include the repressible promoters set forth in SEQ ID NO:885, 856, 857, 858, 859, or 860 or active variants and fragments thereof.

2. Gene Silencing Construct

Another component of the chemical-gene switch disclosed herein comprises a polynucleotide comprising a gene silencing construct. The gene silencing construct encodes a silencing element that decreases the level of the chemically regulated transcriptional repressor. Thus, the presence of the silencing element maintains a state of de-repression. In specific embodiments, the silencing elements are cell non-autonomous, the state of de-repression becomes distributed in plant cells, tissues, organs or throughout the plant, beyond where the chemical ligand physically reaches.

As used herein, the term “cell non-autonomous” in intended that the silencing element initiates a diffusible signal that travels between cells. A cell non-autonomous signal includes both the expansion of the RNA silencing into neighboring plant cells in the form of a “local cell-to-cell” movement or it may occur over longer distances representing “extensive silencing”. Local cell-to-cell movement allows for the signal to spread about 10-15 cells beyond the site of initiation of the expression of the silencing element. This type of spread can occur, but is not limited to, spreading via the plasmodesmata. In other embodiments, the expansion of the silencing into neighboring plant cells results in “extensive silencing”. In such instances, the silencing occurs over distances greater than 10-15 cells from the original cell initiating the signal. In some instances, the signal extends beyond the site of initiation and spreads greater than 15 cells from the initiation site, it spreads throughout a tissue, it spreads throughout an organ, or it spreads systemically through the plant. As used herein, the term “complete penetration” occurs when a sufficient amount of the silencing element is present in a given cell, tissue, organ or entire plant to decrease the level of the chemically-regulated transcriptional repressor to allow for the de-repression of the chemical-gene switch. In still other embodiments, the silencing element is transported by the vasculature of the plant.

Thus, in specific embodiments, the cell non-autonomous silencing element decreases the level of the chemically-regulated transcriptional repressor such that the effective amount of the chemical ligand to the plant results in the spatially or temporally extended expression of the polynucleotide of interest in the plant as compared to expression in a plant having been contacted with the effective amount of the chemical ligand and lacking the gene silencing construct. In some instances, this effect is achieved by providing an amount of chemical ligand smaller than the amount required to induce expression of said polynucleotide of interest in a plant lacking the silencing construct.

By “temporally extending expression” is intended the expression occurs in the absence of the ligand for at least 1, 2, 3, 4, 5, 6, 7 days, 1, 2, 3, 4, 5, 6, 7, 8, 9 months or more, or permanently.

In further embodiments, the expression of the polynucleotide sequence of interest is extended into at least one tissue of the plant which was not contacted by the effective amount of the chemical ligand. In other embodiments, the expression of the polynucleotide of interest is extended such that complete penetration of expression of the polynucleotide of interest in the shoot apical meristem occurs, or such that complete penetration throughout the plant of the expression of the polynucleotide sequence of interest occurs.

a. Target Sequence

As used herein, a “target sequence” comprises any sequence that one desires to decrease the level of expression via expression of the silencing element. Within the context of the chemical-gene switch system disclosed herein, the target sequence comprises the chemically-regulated transcriptional repressor or its 5′ or 3′ UTR sequences.

b. Silencing Element

By “silencing element” is intended a polynucleotide that is capable of decreasing or eliminating the level or expression of a target polynucleotide or the polypeptide encoded thereby. In the methods and compositions provided herein, the silencing element employed can decrease or eliminate the expression level of the chemically-regulated transcriptional repressor sequence by influencing the level of the RNA transcript of the chemically-regulated transcriptional repressor or, alternatively, by influencing translation and thereby affecting the level of the encoded chemically-regulated transcriptional repressor polypeptide. Methods to assay for functional silencing elements that are capable of decreasing or eliminating the level of the chemically-regulated transcriptional repressor are disclosed elsewhere herein. A single polynucleotide employed in the methods of the invention can comprises one or more silencing elements to the same or different chemically-regulated transcriptional repressor.

By “decrease” or “decreasing” the level of a polynucleotide or a polypeptide encoded thereby is intended to mean, the polynucleotide or polypeptide level of the target sequence (i.e., the chemically-regulated transcriptional repressor) is statistically lower than the polynucleotide level or polypeptide level of the same target sequence in an appropriate control plant or tissue which is not exposed to (i.e., has not been exposed to the chemical ligand) the silencing element. In particular embodiments, decreasing the polynucleotide level and/or the polypeptide level of the chemically-regulated transcriptional repressor results in a decrease of at least about 95%, 90%, 80%, 70%, 60%, 50%, 40%, 30%, 20%, 10%, 5%, or 1% of the polynucleotide level, or the level of the polypeptide encoded thereby of the chemically-regulated transcriptional repressor, when compared to an appropriate control (i.e., in the absence of the silencing element or the chemical ligand). Methods to assay for the level of the RNA transcript, the level of the encoded polypeptide, or the activity of the chemically-regulated transcriptional repressor are discussed elsewhere herein.

As discussed in further detail below, silencing elements can include, but are not limited to, a sense suppression element, an antisense suppression element, a double stranded RNA, a miRNA, an amiRNA, or a hairpin suppression element. Non-limiting examples of target sequences include the various chemically-regulated transcriptional repressors discussed elsewhere herein, including the various SuR polypeptides, or polynucleotide encoding the same, such as those set forth in any of SEQ ID NOs:1-836, 863-870, 884-889, 1381-1568 and/or 2030-2110 or active variants and fragments thereof. In some embodiment, the entire chemically-regulated transcriptional repressor, a region comprising the DNA binding domain, a region comprising the ligand binding domain, or the 5′ or 3′ UTR or variants and fragments thereof can be employed in the silencing element.

In specific embodiments, the silencing element comprises at least or consists of 15, 20, 22, 25 or greater consecutive nucleotides encoding a chemically-regulated transcriptional repressor discussed elsewhere herein, including the various SuR polypeptides, such as those set forth in any of SEQ ID NOs:1-836, 863-870, 884-889, 1381-1568 and/or 2030-2110 or active variants and fragments thereof. In other embodiments, the silencing element comprises at least or consists of a polynucleotide encoding amino acids 1-7, 7-14, 14-21, 14-28, 28-35, 35-42, 42-49, 49-56, 56-63, 63-70, 70-77, 77-84, 84-91, 91-98, 98-105, 105-112, 112-119, 119-126, 126-133, 133-140, 140-147, 147-154, 154-161, 161-168, 168-175, 175-182, 182-189, 189-196, 196-203, or 203-207 of a chemically-regulated transcriptional repressors discussed elsewhere herein, including the various SuR polypeptides, such as those set forth in any of SEQ ID NOs:1-836, 863-870, 884-889, 1381-1568 and/or 2030-2110 or active variants and fragments thereof. Alternatively, the silencing element comprises at least or consists of 15, 20, 22, 25 or greater consecutive nucleotides of the 5′ or 3′ untranslated regions (i.e. 5′UTR or 3′ UTR) of the polynucleotide cassette encoding the chemically-regulated transcriptional repressor or a combination of untranslated and coding sequences.

i. Antisense Silencing Elements

As used herein, an “antisense silencing element” comprises a polynucleotide which is designed to express an RNA molecule complementary to all or part of a target messenger RNA. Expression of the antisense RNA suppression element reduces or eliminates the level of the target polynucleotide. The polynucleotide for use in antisense suppression may correspond to all or part of the complement of the sequence encoding the target polynucleotide (i.e., sequence encoding the chemically-regulated transcriptional repressor), all or part of the complement of the 5′ and/or 3′ untranslated region of the target polynucleotide (i.e., sequence encoding the chemically-regulated transcriptional repressor), all or part of the complement of the coding sequence of the target polynucleotide (i.e., sequence encoding the chemically-regulated transcriptional repressor), or all or part of the complement of both the coding sequence and the untranslated regions of the target polynucleotide (i.e., sequence encoding the chemically-regulated transcriptional repressor). In addition, the antisense suppression element may be fully complementary (i.e., 100% identical to the complement of the target sequence) or partially complementary (i.e., less than 100% identical to the complement of the target sequence) to the target polynucleotide. In specific embodiments, the antisense suppression element comprises at least 85%, 90%, 91%, 92%, 93%, 94%, 95%, 96%, 97%, 98%, or 99% sequence identity to the target polynucleotide (i.e., sequence encoding the chemically-regulated transcriptional repressor). Antisense suppression may be used to inhibit the expression of multiple proteins in the same plant. See, for example, U.S. Pat. No. 5,942,657. Furthermore, the antisense suppression element can be complementary to a portion of the target polynucleotide. Generally, sequences of at least 25, 50, 100, 200, 300, 400, 450 nucleotides or greater may be used. Methods for using antisense suppression to inhibit the expression of endogenous genes in plants are described, for example, in Liu et al (2002) Plant Physiol. 129:1732-1743 and U.S. Pat. Nos. 5,759,829 and 5,942,657, each of which is herein incorporated by reference. In specific embodiments, the antisense element comprises or consists of the complement of at least 15, 20, 22, 25 or greater contiguous nucleotides of any one of SEQ ID NO: 1-836, 863-870, 884-889, 1381-1568 and/or 2030-2110.

ii. Double Stranded RNA Silencing Element

A “double stranded RNA silencing element” or “dsRNA” comprises at least one transcript that is capable of forming a dsRNA. Thus, a “dsRNA silencing element” includes a dsRNA, a transcript or polyribonucleotide capable of forming a dsRNA or more than one transcript or polyribonucleotide capable of forming a dsRNA. “Double stranded RNA” or “dsRNA” refers to a polyribonucleotide structure formed either by a single self-complementary RNA molecule or a polyribonucleotide structure formed by the expression of least two distinct RNA strands. The dsRNA molecule(s) employed in the methods and compositions of the invention mediate the reduction of expression of a target sequence (i.e., sequence encoding the chemically-regulated transcriptional repressor), for example, by mediating RNA interference “RNAi” or gene silencing in a sequence-specific manner. In the context of the present invention, the dsRNA is capable of decreasing or eliminating the level or expression of the polypeptide encoded the chemically-regulated transcriptional repressor.

The dsRNA can decrease or eliminate the expression level of the target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, for example, Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional RNAi that are capable of reducing or eliminating the level of a sequence of interest are disclosed elsewhere herein. Accordingly, as used herein, the term “dsRNA” is meant to encompass other terms used to describe nucleic acid molecules that are capable of mediating RNA interference or gene silencing, including, for example, short-interfering RNA (siRNA), double-stranded RNA (dsRNA), hairpin RNA, short hairpin RNA (shRNA), trans-acting siRNA (TAS), post-transcriptional gene silencing RNA (ptgsRNA), and others.

In specific embodiments, at least one strand of the duplex or double-stranded region of the dsRNA shares sufficient sequence identity or sequence complementarity to the polynucleotide encoding the chemically-regulated transcriptional regulator to allow for the dsRNA to reduce the level of expression of the chemically-regulated transcriptional regulator. As used herein, the strand that is complementary to the target polynucleotide is the “antisense strand” and the strand homologous to the target polynucleotide is the “sense strand.”

In one embodiment, the dsRNA comprises a hairpin RNA. A hairpin RNA comprises an RNA molecule that is capable of folding back onto itself to form a double stranded structure. Multiple structures can be employed as hairpin elements. In specific embodiments, the dsRNA suppression element comprises a hairpin element which comprises in the following order, a first segment, a second segment, and a third segment, where the first and the third segment share sufficient complementarity to allow the transcribed RNA to form a double-stranded stem-loop structure.

The “second segment” of the hairpin comprises a “loop” or a “loop region.” These terms are used synonymously herein and are to be construed broadly to comprise any nucleotide sequence that confers enough flexibility to allow self-pairing to occur between complementary regions of a polynucleotide (i.e., segments 1 and 2 which form the stem of the hairpin). For example, in some embodiments, the loop region may be substantially single stranded and act as a spacer between the self-complementary regions of the hairpin stem-loop. In some embodiments, the loop region can comprise a random or nonsense nucleotide sequence and thus not share sequence identity to a target polynucleotide. In other embodiments, the loop region comprises a sense or an antisense RNA sequence or fragment thereof that shares identity to a target polynucleotide. See, for example, International Patent Publication No. WO 02/00904, which is herein incorporated by reference. In specific embodiments, the loop region can be optimized to be as short as possible while still providing enough intramolecular flexibility to allow the formation of the base-paired stem region. In other embodiments, the loop region comprises a spliceable or non-spliceable intron. Accordingly, the loop sequence is generally less than 1000, 900, 800, 700, 600, 500, 400, 300, 200, 100, 50, 25, 20, 15, 10 nucleotides or less.

The “first” and the “third” segment of the hairpin RNA molecule comprise the base-paired stem of the hairpin structure. The first and the third segments are inverted repeats of one another and share sufficient complementarity to allow the formation of the base-paired stem region. In specific embodiments, the first and the third segments are fully complementary to one another. Alternatively, the first and the third segment may be partially complementary to each other so long as they are capable of hybridizing to one another to form a base-paired stem region. The amount of complementarity between the first and the third segment can be calculated as a percentage of the entire segment. Thus, the first and the third segment of the hairpin RNA generally share at least 50%, 60%, 70%, 80%, 85%, 90%, 91%, 92%, 93%, 94%, 95%, 96%, 97%, 98%, 99%, up to and including 100% complementarity.

The first and the third segment are at least about 1000, 500, 400, 300, 200, 100, 50, 40, 30, 25, 22, 20, or 19 nucleotides in length. In specific embodiments, the length of the first and/or the third segment is about 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 20 nucleotides. In other embodiments, the length of the first and/or the third segment comprises at least 10-20 nucleotides, 20-35 nucleotides, 30-45 nucleotides, 40-50 nucleotides, 50-100 nucleotides, or 100-300 nucleotides. See, for example, International Publication No. WO 0200904. In specific embodiments, the first and the third segment comprise at least 20 nucleotides having at least 85% complementary to the first segment. In still other embodiments, the first and the third segments which form the stem-loop structure of the hairpin comprises 3′ or 5′ overhang regions having unpaired nucleotide residues.

In specific embodiments, the sequences used in the first, the second, and/or the third segments comprise domains that are designed to have sufficient sequence identity to a target polynucleotide (i.e., polynucleotide encoding the chemically-regulated transcriptional regulator) and thereby have the ability to decrease the level of the target polynucleotide. The specificity of the inhibitory RNA transcripts is therefore generally conferred by these domains of the silencing element. Thus, in some embodiments of the invention, the first, second and/or third segment of the silencing element comprise a domain having at least 10, at least 15, at least 19, at least 20, at least 21, at least 22, at least 23, at least 24, at least 25, at least 30, at least 40, at least 50, at least 100, at least 200, at least 300, at least 500, at least 1000, or more than 1000 nucleotides that share sufficient sequence identity to the polynucleotide encoding the chemically-regulated transcriptional regulator to allow for a decrease in expression levels of the target polynucleotide when expressed in an appropriate cell (i.e., any one of SEQ ID NO: 1-836, 863-870, 884-889, 1381-1568 and/or 2030-2110 or the polynucleotide encoding the same). In other embodiments, the domain is between about 15 to 50 nucleotides, about 20-35 nucleotides, about 25-50 nucleotides, about 20 to 75 nucleotides, about 40-90 nucleotides about 15-100 nucleotides of the chemically-regulated transcriptional repressor.

In specific embodiments, the domain of the first, the second, and/or the third segment has 100% sequence identity to the polynucleotide encoding the chemically-regulated transcriptional regulator, promoter, 5′ UTR or 3′ UTR. In other embodiments, the domain of the first, the second and/or the third segment having homology to the target polypeptide have at least 50%, 60%, 70%, 80%, 85%, 90%, 91%, 92%, 93%, 94%, 95%, 96%, 97%, 98%, 99%, or greater sequence identity to a region of the polynucleotide encoding the chemically-regulated transcriptional regulator. The sequence identity of the domains of the first, the second and/or the third segments to the target polynucleotide need only be sufficient to decrease expression of the target polynucleotide of interest. See, for example, Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

The amount of complementarity shared between the first, second, and/or third segment and the target polynucleotide or the amount of complementarity shared between the first segment and the third segment (i.e., the stem of the hairpin structure) may vary depending on the plant in which gene expression is to be controlled. Some plants or cell types may require exact pairing or 100% identity, while other plants or cell types may tolerate some mismatching. In some cells, for example, a single nucleotide mismatch in the targeting sequence abrogates the ability to suppress gene expression.

Any region of the polynucleotide encoding the chemically-regulated transcriptional regulator can be used to design the domain of the silencing element that shares sufficient sequence identity to allow expression of the hairpin transcript to decrease the level of the chemically-regulated transcriptional regulator. For instance, the domain can be designed to share sequence identity to the 5′ untranslated region of the polynucleotide encoding the chemically-regulated transcriptional regulator, the 3′ untranslated region of the polynucleotide encoding the chemically-regulated transcriptional regulator, exonic regions of the polynucleotide encoding the chemically-regulated transcriptional regulator, intronic regions of the polynucleotide encoding the chemically-regulated transcriptional regulator, and any combination thereof. In specific embodiments a domain of the silencing element shares sufficient homology to at least about 15 consecutive nucleotides from about nucleotides 1-50, 50-100, 100-150, 150-200, 200-250, 250-300, 300-350, 350-400, 400-450, 450-500, 550-600, 600-650, 650-700, 750-800, 850-900, 950-1000, 1000-1050, 1050-1100, 1100-1200, 1200-1300, 1300-1400, 1400-1500, 1500-1600, 1600-1700, 1700-1800, 1800-1900, 1900-2000 of the polynucleotide encoding the chemically-regulated transcriptional regulator. In some instances to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers et al. (2003) J. Biol. Chem 278:7108-7118 and Yang et al. (2002) Proc. Natl. Acad. Sci. USA 99:9442-9447, herein incorporated by reference. These studies indicate that there is a significant correlation between the RNase-H-sensitive sites and sites that promote efficient siRNA-directed mRNA degradation.

The hairpin silencing element may also be designed such that the sense or the antisense sequence do not correspond to a target polynucleotide. In this embodiment, the sense and antisense sequence flank a loop sequence that comprises a nucleotide sequence corresponding to all or part of the target polynucleotide. Thus, it is the loop region that determines the specificity of the RNA interference. See, for example, WO 02/00904, herein incorporated by reference.

In specific embodiments, the silencing element comprising the hairpin comprises a sequence selected from the group consisting of a polynucleotide comprising or consist of at least one of the sequences of the various chemically-regulated transcriptional repressors discussed elsewhere herein, including the various SuR polypeptides, such as those set forth in any of SEQ ID NOs:1-836, 863-870, 884-889, 1381-1568 and/or 2030-2110 or active variants and fragments thereof. In some embodiments, the entire chemically-regulated transcriptional repressor is employed or only a region comprising the DNA binding domain or a variant or fragment thereof or the ligand binding domain or a variant or fragment thereof is employed in hairpin of the silencing element.

In specific embodiments, the silencing element comprises at least or consists of 15, 20, 22, 25 or greater consecutive nucleotides encoding a chemically-regulated transcriptional repressor discussed elsewhere herein, including the various SuR polypeptides, such as those set forth in any of SEQ ID NOs: 1-836, 863-870, 884-889, 1381-1568 and/or 2030-2110 or active variants and fragments thereof. In other embodiments, the silencing element comprises at least or consists of a polynucleotide encoding amino acids 1-7, 7-14, 14-21, 14-28, 28-35, 35-42, 42-49, 49-56, 56-63, 63-70, 70-77, 77-84, 84-91, 91-98, 98-105, 105-112, 112-119, 119-126, 126-133, 133-140, 140-147, 147-154, 154-161, 161-168, 168-175, 175-182, 182-189, 189-196, 196-203, or 203-207 of a chemically-regulated transcriptional repressors discussed elsewhere herein, including the various SuR polypeptides, such as those set forth in any of SEQ ID NOs:1-836, 863-870, 884-889, 1381-1568 and/or 2030-2110 or active variants and fragments thereof. Alternatively, the silencing element comprises at least or consists of 15, 20, 22, 25 or greater consecutive nucleotides of the 5′ or 3′ translated region of the polynucleotide encoding the chemically-regulated transcriptional repressor or a combination of translated and coding sequences.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz et al. (2002) PNAS 99 (Suppl. 4):16499-16506 and Mette et al. (2000) EMBO J 19(19):5194-5201.

It is envisioned that a trans-acting siRNA (tasiRNA) or microRNA (miRNA) with targeting sequences to the repressor transcript can be substituted for the hairpin cassettes in the above vectors. Likewise different repressors can be substituted as long as the miRNA is modified to new target. In this case the repressor can be that of TetR, or any of the SuR's. While the hairpin approach would potentially target related repressor sequences in the same plant/plant cell, a miRNA could be made to target one specific repressor type. This would enable auto-induction of multiple gene circuits in an independent fashion.

The methods and compositions of the invention employ silencing elements that when transcribed “form” a dsRNA molecule. Accordingly, the heterologous polynucleotide being expressed need not form the dsRNA by itself, but can interact with other sequences in the plant cell to allow the formation of the dsRNA. For example, a chimeric polynucleotide that can selectively silence the target polynucleotide can be generated by expressing a chimeric construct comprising the target sequence for a miRNA or siRNA to a sequence corresponding to all or part of the gene or genes to be silenced. In this embodiment, the dsRNA is “formed” when the target for the miRNA or siRNA interacts with the miRNA present in the cell. The resulting dsRNA can then reduce the level of expression of the gene or genes to be silenced. See, for example, U.S. Application Publication 2007-0130653, herein incorporated by reference. As discussed elsewhere herein, any method can be used to introduce the construct comprising the heterologous miRNA.

(iii) MicroRNA (miRNA) Silencing Element

In other embodiments, the silencing element can comprise a micro RNA (miRNA). “MicroRNAs” or “miRNAs” are regulatory agents comprising about 19 to about 24 ribonucleotides in length, which are highly efficient at inhibiting the expression of target polynucleotides. See, for example Javier et al. (2003) Nature 425: 257-263, herein incorporated by reference. For miRNA interference, the silencing element can be designed to express a dsRNA molecule that forms a hairpin structure containing a 19, 20, 21, 22, 23, 24 or 25 nucleotide sequence that is complementary to the target polynucleotide of interest. The miRNA can be synthetically made, or transcribed as a longer RNA which is subsequently cleaved to produce the active miRNA. The miRNA can be an “artificial miRNA” or “amiRNA” which comprises a miRNA sequence that is synthetically designed to silence a target sequence.

When expressing an miRNA, the final (mature) miRNA is present in a duplex in a precursor backbone structure, the two strands being referred to as the miRNA (the strand that will eventually basepair with the target) and miRNA* (star sequence). It has been demonstrated that miRNAs can be transgenically expressed and target genes of interest efficiently silenced (Highly specific gene silencing by artificial microRNAs in Arabidopsis Schwab et al. (2006) Plant Cell. May; 18(5):1121-33; Epub 2006 Mar. 10 & Expression of artificial microRNAs in transgenic Arabidopsis thaliana confers virus resistance. Niu et al. (2006) Nat Biotechnol. 2006 November; 24(11):1420-8. Epub 2006 Oct. 22. Erratum in: Nat Biotechnol. 2007 February; 25(2):254; each of which are herein incorporated by reference.)

The silencing element for miRNA interference comprises a miRNA precursor backbone. The miRNA precursor backbone comprises a DNA sequence having the miRNA and star sequences. When expressed as an RNA, the structure of the miRNA precursor backbone is such as to allow for the formation of a hairpin RNA structure that can be processed into a miRNA. In some embodiments, the miRNA precursor backbone comprises a genomic miRNA precursor sequence, wherein said sequence comprises a native precursor in which a heterologous (artificial) miRNA and star sequence are inserted.

As used herein, a “star sequence” is the sequence within a miRNA precursor backbone that is complementary to the miRNA and forms a duplex with the miRNA to form the stem structure of a hairpin RNA. In some embodiments, the star sequence can comprise less than 100% complementarity to the miRNA sequence. Alternatively, the star sequence can comprise at least 99%, 98%, 97%, 96%, 95%, 90%, 85%, 80% or lower sequence complementarity to the miRNA sequence as long as the star sequence has sufficient complementarity to the miRNA sequence to form a double stranded structure. In still further embodiments, the star sequence comprises a sequence having 1, 2, 3, 4, 5 or more mismatches with the miRNA sequence and still has sufficient complementarity to form a double stranded structure with the miRNA sequence resulting in production of miRNA and suppression of the target sequence.

The miRNA precursor backbones can be from any plant. In some embodiments, the miRNA precursor backbone is from a monocot. In other embodiments, the miRNA precursor backbone is from a dicot. In further embodiments, the backbone is from maize or soybean. MicroRNA precursor backbones have been described previously. For example, US20090155910A1 (WO 2009/079532) discloses the following soybean miRNA precursor backbones: 156c, 159, 166b, 168c, 396b and 398b, and US20090155909A1 (WO 2009/079548) discloses the following maize miRNA precursor backbones: 159c, 164h, 168a, 169r, and 396h. Each of these references is incorporated by reference in their entirety.

Thus, the miRNA precursor backbone can be altered to allow for efficient insertion of heterologous miRNA and star sequences within the miRNA precursor backbone. In such instances, the miRNA segment and the star segment of the miRNA precursor backbone are replaced with the heterologous miRNA and the heterologous star sequences, designed to target any sequence of interest, using a PCR technique and cloned into an expression construct. It is recognized that there could be alterations to the position at which the artificial miRNA and star sequences are inserted into the backbone. Detailed methods for inserting the miRNA and star sequence into the miRNA precursor backbone are described in, for example, US Patent Applications 20090155909A1 and US20090155910A1, herein incorporated by reference in their entirety.

When designing a miRNA sequence and star sequence, various design choices can be made. See, for example, Schwab R, et al. (2005) Dev Cell 8: 517-27. In non-limiting embodiments, the miRNA sequences disclosed herein can have a “U” at the 5′-end, a “C” or “G” at the 19^{th} nucleotide position, and an “A” or “U” at the 10th nucleotide position. In other embodiments, the miRNA design is such that the miRNA have a high free delta-G as calculated using the ZipFold algorithm (Markham, N. R. & Zuker, M. (2005) Nucleic Acids Res. 33: W577-W581.) Optionally, a one base pair change can be added within the 5′ portion of the miRNA so that the sequence differs from the target sequence by one nucleotide.

c. Promoters for Expression of the Silencing Elements

The polynucleotide encoding the silencing element is operably linked to a repressible promoter active in the plant. Various repressible promoters that can be used to express the silencing element are discussed in detail elsewhere herein.

3. Expression Construct Comprising a Polynucleotide of Interest.

Any polynucleotide of interest can be expressed in the chemical-gene switch disclosed herein. In specific embodiments, expression of the polynucleotide of interest alters the phenotype and/or genotype of the plant. An altered genotype includes any heritable modification to any sequence in a plant genome. An altered phenotype includes any scenario wherein a cell, tissue, plant, and/or seed exhibits a characteristic or trait that distinguishes it from its unaltered state. Altered phenotypes included, but are not limited to, a different growth habit, altered flower color, altered relative maturity, altered yield, altered fertility, altered flowering time, altered disease tolerance, altered insect tolerance, altered herbicide tolerance, altered stress tolerance, altered water tolerance, altered drought tolerance, altered seed characteristics, altered morphology, altered agronomic characteristic, altered metabolism, altered gene expression profile, altered ploidy, altered crop quality, altered forage quality, altered silage quality, altered processing characteristics, and the like.

Polynucleotides of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism, as well as, those affecting kernel size, sucrose loading, and the like.

In still other embodiments, the polynucleotide of interest may be any sequence of interest, including but not limited to sequences encoding a polypeptide, encoding an mRNA, encoding an RNAi precursor, encoding an active RNAi agent, a miRNA, an antisense polynucleotide, a ribozyme, a fusion protein, a replicating vector, a screenable marker, and the like. Expression of the polynucleotide of interest may be used to induce expression of an encoding RNA and/or polypeptide, or conversely to suppress expression of an encoded RNA, RNA target sequence, and/or polypeptide. In specific examples, the polynucleotide sequence may be a polynucleotide encoding a plant hormone, plant defense protein, a nutrient transport protein, a biotic association protein, a desirable input trait, a desirable output trait, a stress resistance gene, a disease/pathogen resistance gene, a male sterility gene, a developmental gene, a regulatory gene, a DNA repair gene, a transcriptional regulatory gene or any other polynucleotide and/or polypeptide of interest.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene); glyphosate (e.g., the EPSPS gene and the GAT gene; see, for example, U.S. Publication No. 20040082770 and WO 03/092360); or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

a. Promoters for Expression of the Polynucleotide of Interest

The polynucleotide of interest is operably linked to a repressible promoter active in the plant. Various repressible promoters that can be used to express the silencing element are discussed in detail elsewhere herein.

4. Promoters

As outlined in detail above, a number of promoters can be used in the various constructs of the chemical-gene switch. The promoters can be selected based on the desired outcome. Promoters of interest can be a constitutive promoter or a non-constitutive promoter. Non-constitutive promoter can include, but are not limited to, a tissue preferred promoter, an inducible promoter, a repressible promoter, a developmental stage preferred promoter, or a promoter having more than one of these properties. In some examples the promoter is primarily expressed in roots, leaves, stems, flowers, silks, anthers, pollen, meristem, germline, seed, endosperm, embryos, or progeny. Non-limiting examples of promoters employed within the constructs of the chemical-gene switch are discussed in detail below.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a 3-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is an endosperm-specific promoter. Globulin 1 (Glb-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed; herein incorporated by reference.

Additional exemplary promoters include but are not limited to a 35S CaMV promoter (Odell et al. (1995) Nature 313:810-812), a S-adenosylmethionine synthase promoter (SAMS) (e.g., those disclosed in U.S. Pat. No. 7,217,858 and US2008/0026466), a Mirabilis mosaic virus promoter (e.g., Dey & Maiti (1999) Plant Mol Biol 40:771-782; Dey & Maiti (1999) Transgenics 3:61-70), an elongation factor promoter (e.g., US2008/0313776 and US2009/0133159), a banana streak virus promoter, an actin promoter (e.g., McElroy et al. (1990) Plant Cell 2:163-171), a TobRB7 promoter (e.g., Yamamoto et al. (1991) Plant Cell 3:371), a patatin promoter (e.g., patatin B33, Martin et al. (1997) Plant J 11:53-62), a ribulose 1,5-bisphosphate carboxylase promoter (e.g., rbcS-3A, see, for example Fluhr et al. (1986) Science 232:1106-1112, and Pellingrinischi et al. (1995) Biochem Soc Trans 23:247-250), an ubiquitin promoter (e.g., Christensen et al. (1992) Plant Mol Biol 18:675-689, and Christensen & Quail (1996) Transgen Res 5:213-218), a metallothionin promoter (e.g., US2010/0064390), a Rab17 promoter (e.g., Vilardell et al. (1994) Plant Mol Biol 24:561-569), a conglycinin promoter (e.g., Chamberland et al. (1992) Plant Mol Biol 19:937-949), a plasma membrane intrinsic (PIP) promoter (e.g., Alexandersson et al. (2009) Plant J 61:650-660), a lipid transfer protein (LTP) promoter (e.g., US2009/0158464, US2009/0070893, and US2008/0295201), a gamma zein promoter (e.g., Uead et al. (1994) Mol Cell Biol 14:4350-4359), a gamma kafarin promoter (e.g., Mishra et al. (2008) Mol Biol Rep 35:81-88), a globulin promoter (e.g., Liu et al. (1998) Plant Cell Rep 17:650-655), a legumin promoter (e.g., U.S. Pat. No. 7,211,712), an early endosperm promoter (EEP) (e.g., US2007/0169226 and US2009/0227013), a B22E promoter (e.g., Klemsdal et al. (1991) Mol Gen Genet 228:9-16), an oleosin promoter (e.g., Plant et al. (1994) Plant Mol Biol 25:193-205), an early abundant protein (EAP) promoter (e.g., U.S. Pat. No. 7,321,031), a late embryogenesis abundant (LEA) protein (e.g., Hva1, Straub et al. (1994) Plant Mol Biol 26:617-630; Dhn and WSI18, Xiao & Xue (2001) Plant Cell Rep 20:667-673), In2-2 promoter (De Veylder et al. (1997) Plant Cell Physiol 38:568-577), a glutathione S-transferase (GST) promoter (e.g., WO93/01294), a PR promoter (e.g., Cao et al. (2006) Plant Cell Rep 6:554-560, and Ono et al. (2004) Biosci Biotech Biochem 68:803-807), an ACE1 promoter (e.g., Mett et al. (1993) Proc Natl Acad Sci USA 90:4567-4571), a steroid responsive promoter (e.g., Schena et al. (1991) Proc Natl Acad Sci USA 88:10421-10425, and McNellis et al. (1998) Plant J 14:247-257), an ethanol-inducible promoter (e.g., A1cA, Caddick et al. (1988) Nat Biotechnol 16:177-180), an estradiol-inducible promoter (e.g., Bruce et al. (2000) Plant Cell 12:65-79), an XVE estradiol-inducible promoter (e.g., Zao et al. (2000) Plant J 24: 265-273), a VGE methoxyfenozide-inducible promoter (e.g., Padidam et al. (2003) Transgen Res 12:101-109), or a TGV dexamethasone-inducible promoter (e.g., Bohner et al. (1999) Plant J 19:87-95).

a. Repressible Promoters

As used herein, a “repressible promoter” comprises at least one operator sequence to which the chemically-regulated transcriptional repressor polypeptide specifically binds, and thereby controls the transcriptional activity of the promoter. In the absence of a repressor, the repressible promoter is active and will initiate transcription of an operably linked polynucleotide. In the presence of the repressor, the repressor will bind to the operator sequence and represses transcription. Within the context of the chemical-gene switch, the repressor comprises the chemically-regulated transcriptional repressor, and the chemical ligand influences if it can bind or not bind to the operator. Thus, the binding of the repressor to the operator will be influenced by the presence or absence of a chemical ligand, such that the presence of the chemical ligand will block the transcriptional repressor from binding to the operator. A promoter with “repressible promoter activity” will direct expression of an operably linked polynucleotide, wherein its ability to direct transcription depends on the presence or absence of a chemical ligand (i.e., a tetracycline compound, a sulfonylurea compound) and a corresponding chemically-regulated transcriptional repressor protein. Thus, the presence of the operator “regulates” transcription (increase or decreases expression) of the operably linked sequence.

Any combination of promoters and operators may be employed to form a repressible promoter. Operators of interest include, but are not limited to, the Tet operator sequence is a TetR(A) operator sequence, a TetR(B) operator sequence, a TetR(D) operator sequence, TetR(E) operator sequence, a TetR(H) operator sequence, or an active variant or fragment thereof. Additional operators of interest include, but are not limited to, those that are regulated by the following repressors: tet, lac, trp, phd, arg, LexA, phiCh1 repressor, lambda C1 and Cro repressors, phage X repressor, MetJ, phir1t rro, phi434 C1 and Cro repressors, RafR, gal, ebg, uxuR, exuR, ROS, SinR, PurR, FruR, P22 C2, TetC, AcrR, Betl, Bm3R1, EnvR, QacR, MtrR, TcmR, Ttk, YbiH, YhgD, and mu Ner, or DNA binding domains in Interpro families including but not limited to IPR001647, IPR010982, and IPR011991.

In one embodiment, the repressible promoter comprises at least one tet operator sequence. Repressors include tet repressors and sulfonylurea-regulated repressors. Binding of a tet repressor to a tet operator is regulated by tetracycline compounds and analogs thereof. Binding of a sulfonylurea-responsive repressor to a tet operator is controlled by sulfonylurea compounds and analogs thereof. The tet operator sequence can be located within 0-30 nucleotides 5′ or 3′ of the TATA box of the repressible promoter, including, for example, within 20, 19, 18, 17, 16, 15, 14, 13, 12, 11, 10, 9, 8, 7, 6, 5, 4, 3, 2, 1, or 0 nt of the TATA box. In other instances, the tet operator sequence may partially overlap with the TATA box sequence. In one non-limiting example, the tet operator sequence is SEQ ID NO:848 or an active variant or fragment thereof.

Useful tet operator containing promoters include, for example, those known in the art (see, e.g., Padidam (2003) Curr Op Plant Biol 6:169-177; Gatz & Quail (1988) PNAS 85:1394-1397; Ulmasov et al. (1997) Plant Mol Biol 35:417-424; Weinmann et al. (1994) Plant J 5:559-569). One or more tet operator sequences can be added to a promoter in order to produce a tetracycline inducible promoter. See, for example, Weinmann et al. (1994) Plant J 5:559-569; Love et al. (2000) Plant J 21:579-588. In addition, a widely tested tetracycline regulated expression system for plants using the CaMV 35S promoter was developed (Gatz et al. (1992) Plant J 2:397-404) having three tet operators introduced near the TATA box (3XOpT 35S).

Thus, a repressible promoter comprising at least one, two, three or more operators (including a tet operator, such as that set forth in SEQ ID NO:848 or an active variant or fragment thereof) regulating expression of said repressor can be used. Non-limiting repressible promoters for expression of the chemically-regulated transcriptional repressor, include the repressible promoters set forth in SEQ ID NO:885, 856, 857, 858, 859, or 860 or active variants and fragments thereof.

Any promoter can be combined with an operator to generate a repressible promoter. In specific embodiments, the promoter is active in plant cells. The promoter can be a constitutive promoter or a non-constitutive promoter. Non-constitutive promoters include tissue-preferred promoter, such as a promoter that is primarily expressed in roots, leaves, stems, flowers, silks, anthers, pollen, meristem, seed, endosperm, or embryos.

In particular embodiments, the promoter is a plant actin promoter, a banana streak virus promoter (BSV), an MMV promoter, an enhanced MMV promoter (dMMV), a plant P450 promoter, or an elongation factor 1a (EF1A) promoter. Promoters of interest include, for example, a plant actin promoter (SEQ ID NO:849), a banana streak virus promoter (BSV) (SEQ ID NO:850), a mirabilis mosaic virus promoter (MMV) (SEQ ID NO:851), an enhanced MMV promoter (dMMV) (SEQ ID NO:852), a plant P450 promoter (MP1) (SEQ ID NO:853), or an elongation factor 1a (EF1A) promoter (SEQ ID NO:854), or an active variant for fragment thereof.

The repressible promoter can comprise one or more operator sequences. For example, the repressible promoter can comprises 1, 2, 3, 4, 5 or more operator sequences. In one embodiment, the repressible promoter comprises two tet operator sequences, wherein the 1^{st} tet operator sequence is located within 0-30 nt 5′ of the TATA box and the 2^{nd} tet operator sequence is located within 0-30 nt 3′ of the TATA box. In some examples, the first and/or the second tet operator sequence is located within 20, 19, 18, 17, 16, 15, 14, 13, 12, 11, 10, 9, 8, 7, 6, 5, 4, 3, 2, 1, or 0 nt of the TATA box. In some examples the first and/or the second tet operator sequence may partially overlap with the TATA box sequence. In some examples, the first and/or the second tet operator sequence is SEQ ID NO:848 or an active variant or fragment thereof.

In other embodiments, the repressible promoter comprises three tet operator sequences, wherein the 1^{st} tet operator sequence is located within 0-30 nt 5′ of the TATA box, and the 2^{nd} tet operator sequence is located within 0-30 nt 3′ of the TATA box, and the 3^{rd} tet operator is located with 0-50 nt of the transcriptional start site (TSS). In some examples, the 1^{st} and/or the 2^{nd} tet operator sequence is located within 20, 19, 18, 17, 16, 15, 14, 13, 12, 11, 10, 9, 8, 7, 6, 5, 4, 3, 2, 1, or 0 nt of the TATA box. In other instances, the 3^{rd} tet operator sequence is located within 50, 45, 40, 35, 30, 25, 20, 19, 18, 17, 16, 15, 14, 13, 12, 11, 10, 9, 8, 7, 6, 5, 4, 3, 2, 1, or 0 nt of the TSS. In some examples, the 3^{rd} tet operator is located 5′ of the TSS, or the 3^{rd} tet operator sequence may partially overlap with the TSS sequence. In one non-limiting embodiment, the 1^{st}, 2^{nd} and/or the 3^{rd} tet operator sequence is SEQ ID NO:848 or active variant or fragment thereof.

In another embodiment the repressible promoter may have a single operator site located proximal to the transcription start site. The 35S promoter can be repressed by having an operator sequence located just downstream of the TSS (Heins et al. (1992) Mol Gen Genet 232:328-331.

In specific examples, the repressible promoter is a plant actin promoter (actin/Op) (SEQ ID NO:855), a banana streak virus promoter (BSV/Op) (SEQ ID NO:856), a mirabilis mosaic virus promoter (MMV/Op) (SEQ ID NO:857), an enhanced MMV promoter (dMMV/Op) (SEQ ID NO:858), a plant P450 promoter (MP1/Op) (SEQ ID NO:859), or an elongation factor 1a (EF1A/Op) promoter (SEQ ID NO:860) or an active variant or fragment thereof. Thus, the repressible promoter can comprise a polynucleotide sequence having at least about 50%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:885, 856, 857, 858, 859, or 860, wherein the promoter retains repressible promoter activity. In a specific example, the promoter comprises a polynucleotide sequence having at least 95% sequence identity to SEQ ID NO:885, 856, 857, 858, 859, or 860, wherein the promoter retains repressible promoter activity.

In some embodiments, the repressible promoter employed in the chemical-gene switch is expressed in various tissues or cells, restricted to selected tissue or cell type, restricted to specific developmental stage(s), restricted to specific environmental conditions, and/or restricted to specific generation of a plant or progeny thereof. In some examples, the polynucleotide of interest operably linked to a repressible promoter that, when un-repressed, expresses primarily expressed in roots, leaves, stems, flowers, silks, anthers, pollen, meristem, germline, seed, endosperm, embryos, or progeny. In some examples, expression of the polynucleotide of interest operably linked to the repressible promoter results in expression occurring primarily at specific times, which include but are not limited to seed or plant developmental stages, vegetative growth, reproductive cycle, response to environmental conditions, response to pest or pathogen presence, response to chemical compounds, or any combination thereof. In other embodiments, expression of the polynucleotide of interest is reduced, inhibited, or blocked in various tissues or cells, which may be restricted to selected tissue or cell type, restricted to specific developmental stage(s), restricted to specific environmental conditions, and/or restricted to specific generation of a plant or progeny thereof. In some examples expression of the polynucleotide of interest is primarily inhibited in roots, leaves, stems, flowers, silks, anthers, pollen, meristem, germline, seed, endosperm, embryos, or progeny. In some examples expression of the polynucleotide of interest occurs primarily inhibited at specific times, which include but are not limited to seed or plant developmental stages, vegetative growth, reproductive cycle, response to environmental conditions, response to pest or pathogen presence, response to chemical compounds, or any combination thereof.

5. Sequence that Confers Tolerance to Chemical Ligand

As discussed in detail above, a variety of chemical ligands and their corresponding chemically-regulated transcriptional repressors can be used in the methods and compositions disclosed herein to assemble the gene switch. It is recognized that the plant or plant part when exposed to the chemical ligand should remain tolerant to the chemical ligand employed. As used herein, “chemical ligand-tolerant” or “tolerant” or “crop tolerance” or “herbicide-tolerant” or “sulfonylurea-tolerant” in the context of chemical-ligand treatment is intended that a plant treated with the chemical ligand of the particular chemical-gene switch system being employed will show no significant damage following the treatment in comparison to a plant or plant part not exposed the chemical ligand. The chemical ligand employed may be a compound which causes no negative effects on the plant. Alternatively, a plant may be naturally tolerant to a particular chemical ligand, or a plant may be tolerant to the chemical ligand as a result of human intervention such as, for example, by the use of a recombinant construct, plant breeding or genetic engineering.

In one embodiment, the chemical-gene switch comprises a chemically-regulated transcriptional repressor comprising a Su(R) polypeptide and the chemical ligand comprises a sulfonylurea compound. When such a chemical-gene switch is employed, the plant containing the chemical-gene switch components should have tolerance to the sulfonylurea compound employed as the chemical ligand. The plants employed with such a chemical-gene switch system can comprise a native or a heterologous sequence that confers tolerance to the sulfonylurea compound.

In one embodiment, the plant comprises a sulfonylurea-tolerant polypeptide. As used herein, a “sulfonylurea-tolerant polypeptide” comprises any polypeptide which when expressed in a plant confers tolerance to at least one sulfonylurea. Sulfonylurea herbicides inhibit growth of higher plants by blocking acetolactate synthase (ALS), also known as, acetohydroxy acid synthase (AHAS). Plants containing particular mutations in ALS (e.g., the S4 and/or HRA mutations) are tolerant to sulfonylurea herbicides. The production of sulfonylurea-tolerant plants is described more fully in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270, which are incorporated herein by reference in their entireties for all purposes. The sulfonylurea-tolerant polypeptide can be encoded by, for example, the SuRA or SuRB locus of ALS. In specific embodiments, the ALS inhibitor-tolerant polypeptide comprises the C3 ALS mutant, the HRA ALS mutant, the S4 mutant or the S4/HRA mutant or any combination thereof. Different mutations in ALS are known to confer tolerance to different herbicides and groups (and/or subgroups) of herbicides; see, e.g., Tranel and Wright (2002) Weed Science 50:700-712. See also, U.S. Pat. Nos. 5,605,011, 5,378,824, 5,141,870, and 5,013,659, each of which is herein incorporated by reference in their entirety. The HRA mutation in ALS finds particular use in one embodiment. The mutation results in the production of an acetolactate synthase polypeptide which is resistant to at least one sulfonylurea compound in comparison to the wild-type protein.

A chemical ligand does not “significantly damage” a plant when it either has no effect on a plant or when it has some effect on a plant from which the plant later recovers, or when it has an effect which is detrimental but which is offset, for example, by the impact of the particular herbicide on weeds or the desired phenotype produced by the chemical-gene switch system. Thus, for example, a plant is not “significantly damaged by” a chemical ligand treatment if it exhibits less than 50%, 40%, 30%, 25%, 20%, 15%, 10%, 9%, 8%, 7%, 6%, 5%, 4%, 3%, 2%, or 1% decrease in at least one suitable parameter that is indicative of plant health and/or productivity in comparison to an appropriate control plant (e.g., an untreated crop plant). Suitable parameters that are indicative of plant health and/or productivity include, for example, plant height, plant weight, leaf length, time elapsed to a particular stage of development, flowering, yield, seed production, and the like. The evaluation of a parameter can be by visual inspection and/or by statistical analysis of any suitable parameter. Comparison may be made by visual inspection and/or by statistical analysis. Accordingly, a crop plant is not “significantly damaged by” a herbicide or other treatment if it exhibits a decrease in at least one parameter but that decrease is temporary in nature and the plant recovers fully within 1 week, 2 weeks, 3 weeks, 4 weeks, or 6 weeks.

### III. Plants

Plants, plant cells, plant parts and seeds, and grain having one or more of the chemical-gene switch components (i.e., the silencing element construct, the polynucleotide sequence of interest construct, and/or the chemically-regulated transcriptional repressor construct) are provided. In specific embodiments, the plants and/or plant parts have stably incorporated at least one of the chemical-gene switch components (i.e., the silencing element construct, the polynucleotide sequence of interest construct, and/or the chemically-regulated transcriptional repressor construct).

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

One or more of the chemical-gene switch components (i.e., the silencing element construct, the polynucleotide sequence of interest construct, and the chemically-regulated transcriptional repressor construct) may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis), and Poplar and Eucalyptus. In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

A “subject plant or plant cell” is one in which genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest and/or the silencing element; (e) the subject plant or plant cell itself, under conditions in which the gene of interest is not expressed.

As outlined above, plants and plant parts having the chemical-gene switch can further display tolerance to the chemical ligand. The tolerance to the chemical ligand can be naturally occurring or can be generated by human intervention via breeding or the introduction of recombination sequences that confer tolerance to the chemical ligand. Thus, in some instances the plants comprising the chemical-gene switch comprise sequence that confer tolerant to an SU herbicide, including for example altered forms of AHAS, including the HRA sequence.

### IV. Polynucleotide Constructs

The use of the term “polynucleotide” is not intended to limit the methods and compositions to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The various comments of the chemical-gene switch system (i.e., the chemically-regulated transcriptional repressor, the silencing element and the polynucleotide of interest and, if needed, the polynucleotide conferring tolerance to the chemical ligand) can be provided in expression cassettes for expression in the plant of interest. The cassette can include 5′ and 3′ regulatory sequences operably linked to the chemically-regulated transcriptional repressor, the silencing element and the polynucleotide of interest. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide (i.e., the chemically-regulated transcriptional repressor, the silencing element and the polynucleotide of interest) to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a component of the chemical-gene switch (i.e., the chemically-regulated transcriptional repressor, the silencing element and the polynucleotide of interest), and a transcriptional and translational termination region (i.e., termination region) functional in plants. The regulatory regions (i.e., promoters, transcriptional regulatory regions, and translational termination regions) and/or the chemically-regulated transcriptional repressor, the silencing element and the polynucleotide of interest may be native/analogous to the host cell or to each other. Alternatively, the regulatory regions may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, may be native with the plant host, or may be derived from another source (i.e., foreign or heterologous) to the promoter, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides of the chemical-gene switch system (i.e., the chemically-regulated transcriptional repressor, the silencing element and the polynucleotide of interest) may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385. See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As discussed in detail elsewhere herein, a number of promoters can be used to express the various components of the chemical-gene switch system. The promoters can be selected based on the desired outcome.

The expression cassette(s) can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glyphosate, glufosinate ammonium, bromoxynil, sulfonylureas, dicamba, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting.

The various components can be introduced into a plant on a single polynucleotide construct or single plasmid or on separate polynucleotide constructs or on separate plasmids. It is further recognized the various components of the gene-switch can be brought together through any means including the introduction of one or more component into a plant and then breeding the individual components together into a single plant.

### V. Methods of Introducing

Various methods can be used to introduce the various components of the chemical-gene switch system in a plant or plant part. “Introducing” is intended to mean presenting to the plant, plant cell or plant part the polynucleotide or polypeptide in such a manner that the sequence gains access to the interior of a cell of the plant. The methods of the invention do not depend on a particular method for introducing a sequence into a plant or plant part, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In other embodiments, the various components of the chemical-gene switch system may be introduced into plants by contacting plants with a virus or viral nucleic acids. Generally, such methods involve incorporating a nucleotide construct of the invention within a DNA or RNA molecule. Methods for introducing polynucleotides into plants and expressing the same, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of one or more of the components of the chemical-gene switch system is achieved using a site-specific recombination system. See, for example, WO99/25821, WO99/25854, WO99/25840, WO99/25855, and WO99/25853, all of which are herein incorporated by reference. Other methods to target polynucleotides are set forth in WO 2009/114321 (herein incorporated by reference), which describes “custom” meganucleases produced to modify plant genomes, in particular the genome of maize. See, also, Gao et al. (2010) Plant Journal 1:176-187.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having one or more of the components of the chemical-gene switch system or all of the components of the chemical-gene switch system, for example, stably incorporated into their genome.

In some examples, the components of the chemical-gene switch system can be introduced into a plastid, either by transformation of the plastid or by directing a SuR transcript or polypeptide into the plastid. Any method of transformation, nuclear or plastid, can be used, depending on the desired product and/or use. Plastid transformation provides advantages including high transgene expression, control of transgene expression, ability to express polycistronic messages, site-specific integration via homologous recombination, absence of transgene silencing and position effects, control of transgene transmission via uniparental plastid gene inheritance and sequestration of expressed polypeptides in the organelle which can obviate possible adverse impacts on cytoplasmic components (e.g., see, reviews including Heifetz (2000) Biochimie 82:655-666; Daniell et al. (2002) Trends Plant Sci 7:84-91; Maliga (2002) Curr Op Plant Biol 5:164-172; Maliga (2004) Ann Rev Plant Biol 55-289-313; Daniell et al. (2005) Trends Biotechnol 23:238-245 and Verma and Daniell (2007) Plant Physiol 145:1129-1143).

Methods and compositions of plastid transformation are well known, for example, transformation methods include (Boynton et al. (1988) Science 240:1534-1538; Svab et al. (1990) Proc Natl Acad Sci USA 87:8526-8530; Svab et al. (1990) Plant Mol Biol 14:197-205; Svab et al. (1993) Proc Natl Acad Sci USA 90:913-917; Golds et al. (1993) Bio/Technology 11:95-97; O'Neill et al. (1993) Plant J 3:729-738; Koop et al. (1996) Planta 199:193-201; Kofer et al. (1998) In Vitro Plant 34:303-309; Knoblauch et al. (1999) Nat Biotechnol 17:906-909); as well as plastid transformation vectors, elements, and selection (Newman et al. (1990) Genetics 126:875-888; Goldschmidt-Clermont, (1991) Nucl Acids Res 19:4083-4089; Caner et al. (1993) Mol Gen Genet 241:49-56; Svab et al. (1993) Proc Natl Acad Sci USA 90:913-917; Verma and Daniell (2007) Plant Physiol 145:1129-1143).

Methods and compositions for controlling gene expression in plastids are well known including (McBride et al. (1994) Proc Natl Acad Sci USA 91:7301-7305; Lössl et al. (2005) Plant Cell Physiol 46:1462-1471; Heifetz (2000) Biochemie 82:655-666; Surzycki et al. (2007) Proc Natl Acad Sci USA 104:17548-17553; U.S. Pat. Nos. 5,576,198 and 5,925,806; WO 2005/0544478), as well as methods and compositions to import polynucleotides and/or polypeptides into a plastid, including translational fusion to a transit peptide (e.g., Comai et al. (1988) J Biol Chem 263:15104-15109).

The SuR polynucleotides and polypeptides provide a means for regulating plastid gene expression via a chemical ligand that readily enters the cell. For example, using the T7 expression system for chloroplasts (McBride et al. (1994) Proc Natl Acad Sci USA 91:7301-7305) the SuR could be used to control nuclear expression of plastid targeted T7 polymerase. Alternatively, a SuR-regulated promoter could be integrated into the plastid genome and operably linked to the polynucleotide(s) of interest and the SuR expressed and imported from the nuclear genome, or integrated into the plastid. In all cases, application of a sulfonylurea compound is used to efficiently regulate the polynucleotide(s) of interest and the silencing element.

### VI. Methods of Using the Chemical-Gene Switch System

Methods to regulate expression in a plant, plant organ or plant tissue are provided. The methods comprise providing a plant comprising (i) a first polynucleotide construct comprising a polynucleotide encoding a chemically-regulated transcriptional repressor operably linked to a promoter active in the plant, (ii) a second polynucleotide construct comprising a polynucleotide of interest operably linked to a first repressible promoter, and (iii) a third polynucleotide construct comprising a gene silencing construct operably linked to a second repressible promoter, wherein the gene silencing construct encodes a silencing element that decreases the level of the chemically-regulated transcriptional repressor. In specific embodiments, silencing element is a non-autonomous silencing element. The first and second repressible promoters each comprise at least one operator, wherein the chemically-regulated transcriptional repressor can bind to each of the operators in the absence of a chemical ligand and thereby repress transcription from the first and the second repressible promoters in the absence of the chemical ligand, and wherein the plant is tolerant to the chemical ligand. The plant is then contacted with an effective amount of the chemical ligand whereby the effective amount of the chemical ligand results in (i) an increase in expression of the polynucleotide of interest and the silencing construct and (ii) a decrease in the level of the chemically-regulated transcriptional repressor. In non-limiting embodiments, the method employs a repressible promoter comprising at least one tetracycline operator in combination with a TetR polypeptide and a ligand comprising a tetracycline compound or an active derivative thereof. In other embodiments, the method employs a repressible promoter comprising at least one tetracycline operator sequence in combination with a SuR polypeptide having a tet operator binding domain and a chemical ligand comprising a sulfonylurea compound.

Any chemical ligand can be employed in the methods, so long as the ligand is compatible with the chemical-gene switch contained in the plant. Chemical ligands include, but are not limited to, tetracycline (when a tetracycline transcriptional repressor is used), or a sulfonylurea (when a Su(R) is employed).

When the chemically-regulated transcription repressor comprises a SuR, then the chemical ligand comprises a sulfonylurea compound. Sulfonylurea molecules comprise a sulfonylurea moiety (—S(O)2NHC(O)NH(R)—). In sulfonylurea herbicides the sulfonyl end of the sulfonylurea moiety is connected either directly or by way of an oxygen atom or an optionally substituted amino or methylene group to a typically substituted cyclic or acyclic group. At the opposite end of the sulfonylurea bridge, the amino group, which may have a substituent such as methyl (R being CH₃) instead of hydrogen, is connected to a heterocyclic group, typically a symmetric pyrimidine or triazine ring, having one or two substituents such as methyl, ethyl, trifluoromethyl, methoxy, ethoxy, methylamino, dimethylamino, ethylamino and the halogens. Sulfonylurea herbicides can be in the form of the free acid or a salt. In the free acid form the sulfonamide nitrogen on the bridge is not deprotonated (i.e., —S(O)2NHC(O)NH(R)—), while in the salt form the sulfonamide nitrogen atom on the bridge is deprotonated, and a cation is present, typically of an alkali metal or alkaline earth metal, most commonly sodium or potassium. Sulfonylurea compounds include, for example, compound classes such as pyrimidinylsulfonylurea compounds, triazinylsulfonylurea compounds, thiadiazolylurea compounds, and pharmaceuticals such as antidiabetic drugs, as well as salts and other derivatives thereof. Examples of pyrimidinylsulfonylurea compounds include amidosulfuron, azimsulfuron, bensulfuron, bensulfuron-methyl, chlorimuron, chlorimuron-ethyl, cyclosulfamuron, ethoxysulfuron, flazasulfuron, flucetosulfuron, flupyrsulfuron, flupyrsulfuron-methyl, foramsulfuron, halosulfuron, halosulfuron-methyl, imazosulfuron, mesosulfuron, mesosulfuron-methyl, nicosulfuron, orthosulfamuron, oxasulfuron, primisulfuron, primisulfuron-methyl, pyrazosulfuron, pyrazosulfuron-ethyl, rimsulfuron, sulfometuron, sulfometuron-methyl, sulfosulfuron, trifloxysulfuron and salts and derivatives thereof. Examples of triazinylsulfonylurea compounds include chlorsulfuron, cinosulfuron, ethametsulfuron, ethametsulfuron-methyl, iodosulfuron, iodosulfuron-methyl, metsulfuron, metsulfuron-methyl, prosulfuron, thifensulfuron, thifensulfuron-methyl, triasulfuron, tribenuron, tribenuron-methyl, triflusulfuron, triflusulfuron-methyl, tritosulfuron and salts and derivatives thereof. Examples of thiadiazolylurea compounds include buthiuron, ethidimuron, tebuthiuron, thiazafluron, thidiazuron, pyrimidinylsulfonylurea compound (e.g., amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulftiron, pyrazosulfuron, rimsulfuron, sulfometuron, sulfosulfuron and trifloxysulfuron); a triazinylsulfonylurea compound (e.g., chlorsulfuron, cinosulfuron, ethametsulfuron, iodosulfuron, metsulfuron, prosulfuron, thifensulfuron, triasulfuron, tribenuron, triflusulfuron and tritosulfuron); or a thiadazolylurea compound (e.g., cloransulam, diclosulam, florasulam, flumetsulam, metosulam, and penoxsulam) and salts and derivatives thereof. Examples of antidiabetic drugs include acetohexamide, chlorpropamide, tolbutamide, tolazamide, glipizide, gliclazide, glibenclamide (glyburide), gliquidone, glimepiride and salts and derivatives thereof. In some systems, the SuR polypeptides specifically bind to more than one sulfonylurea compound, so one can chose which chemical ligand to apply to the plant.

In some examples, the sulfonylurea compound is selected from the group consisting of chlorsulfuron, ethametsulfuron-methyl, metsulfuron-methyl, thifensulfuron-methyl, sulfometuron-methyl, tribenuron-methyl, chlorimuron-ethyl, nicosulfuron, and rimsulfuron.

In other embodiments, the sulfonylurea compound comprises a pyrimidinylsulfonylurea, a triazinylsulfonylurea, a thiadazolylurea, a chlorosulfuron, an ethametsulfuron, a thifensulfuron, a metsulfuron, a sulfometuron, a tribenuron, a chlorimuron, a nicosulfuron, or a rimsulfuron compound.

In some embodiments, the sulfonylurea compound is an ethametsulfuron. In some examples the ethametsulfuron is provided at a concentration of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20 μg/ml or greater applied as a tissue or root drench. Alternatively, the SU compound can be provided by spray at 1-400% of registered label application rates depending on the herbicide product. In some examples, the SuR polypeptide which employs the ethametsulfuron as a chemical ligand comprises a ligand binding domain having at least 50%60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to a SuR polypeptide of SEQ ID NO:205-419, wherein the sequence identity is determined over the full length of the polypeptide using a global alignment method. In some examples the global alignment method is GAP, wherein the default parameters are for an amino acid sequence % identity and % similarity using a GAP Weight of 8 and a Length Weight of 2, and the BLOSUM62 scoring matrix. In some examples the polypeptide has a ligand binding domain from a SuR polypeptide selected from the group consisting of SEQ ID NO:205-419. In some examples the polypeptide is selected from the group consisting of SEQ ID NO:205-419. In some examples the polypeptide is encoded by a polynucleotide of SEQ ID NO:622-836.

In other embodiments, the sulfonylurea compound is chlorsulfuron. In some examples, the chlorsulfuron is provided at a concentration of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20. In some examples the SuR polypeptide which employs the chlorsulfuron as a chemical ligand has a ligand binding domain having at least 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to a SuR polypeptide of SEQ ID NO:14-204, wherein the sequence identity is determined over the full length of the polypeptide using a global alignment method. In some examples the global alignment method is GAP, wherein the default parameters are for an amino acid sequence % identity and % similarity using GAP Weight of 8 and Length Weight of 2 and the BLOSUM62 scoring matrix. In some examples the polypeptide has a ligand binding domain from a SuR polypeptide selected from the group consisting of SEQ ID NO:14-204. In some examples the polypeptide is selected from the group consisting of SEQ ID NO:14-204. In some examples, the polypeptide is encoded by a polynucleotide of SEQ ID NO:431-621.

By “contacting” or “providing to the plant or plant part” is intended any method whereby an effective amount of the chemical ligand is exposed to the plant, plant part, tissue or organ. The chemical ligand can be applied to the plant or plant part by, for example, spraying, atomizing, dusting, scattering, coating or pouring, introducing into or on the soil, introducing into irrigation water, by seed treatment or general application or dusting at the desirable time for the purpose at hand.

By “effective amount” of the chemical ligand is intended an amount of chemical ligand that is sufficient to allow for the desirable level of expression of the polynucleotide sequence of interest in a desired tissue or plant part. Generally, the effective amount of chemical ligand is sufficient to induce or increase expression of the polynucleotide of interest in the desired tissues in the plant, without significantly affecting the plant/crop. When the chemical ligand comprises a sulfonylurea, the effective amount may or may not be sufficient to control weeds. When desired, the expression of the polynucleotide of interest alters the phenotype and/or the genome of the plant.

In specific embodiments, contacting the effective amount of the chemical ligand to the plant results in a spatially or temporally extended expression of the polynucleotide of interest in the plant as compared to expression in a plant having been contacted with the effective amount of said chemical ligand and lacking the gene silencing construct. In some embodiments, the spatially or temporally extended expression of the polynucleotide of interest is achieved by providing an amount of chemical ligand smaller than the amount required to induce expression of the polynucleotide of interest in a plant lacking the gene silencing construct.

The spatially extended expression of the polynucleotide of interest can comprise the expression in at least one tissue of said plant not penetrated by the effective amount of the chemical ligand. In other embodiments, providing the chemical ligand results in the complete penetration of expression of the polynucleotide of interest in the shoot apical meristem of the plant or complete penetration of expression throughout the plant.

In a non-limiting embodiment, the method employs a first repressible promoter operably linked to the polynucleotide of interest, wherein the first repressible promoter comprises at least one, two, three or more operators. The silencing element is operably linked to a second repressible promoter comprising at least one, two, three or more operators, and the promoter operably linked to the chemically-regulated transcriptional repressor comprises a third repressible promoter, wherein the third repressible promoter comprises at least one, two or three or more operators regulating expression of the chemically-regulated transcriptional repressor.

The chemical ligand can be contacted to the plant in combination with an adjuvant or any other agent that provides a desired agricultural effect. As used herein, an “adjuvant” is any material added to a spray solution or formulation to modify the action of an agricultural chemical or the physical properties of the spray solution. See, for example, Green and Foy (2003) “Adjuvants: Tools for Enhancing Herbicide Performance,” in Weed Biology and Management, ed. Inderjit (Kluwer Academic Publishers, The Netherlands). Adjuvants can be categorized or subclassified as activators, acidifiers, buffers, additives, adherents, antiflocculants, antifoamers, defoamers, antifreezes, attractants, basic blends, chelating agents, cleaners, colorants or dyes, compatibility agents, cosolvents, couplers, crop oil concentrates, deposition agents, detergents, dispersants, drift control agents, emulsifiers, evaporation reducers, extenders, fertilizers, foam markers, formulants, inerts, humectants, methylated seed oils, high load COCs, polymers, modified vegetable oils, penetrators, repellants, petroleum oil concentrates, preservatives, rainfast agents, retention aids, solubilizers, surfactants, spreaders, stickers, spreader stickers, synergists, thickeners, translocation aids, uv protectants, vegetable oils, water conditioners, and wetting agents.

In addition, methods of the invention can comprise the use of a herbicide or a mixture of herbicides, as well as, one or more other insecticides, fungicides, nematocides, bactericides, acaricides, growth regulators, chemosterilants, semiochemicals, repellents, attractants, pheromones, feeding stimulants or other biologically active compounds or entomopathogenic bacteria, virus, or fungi to form a multi-component mixture giving an even broader spectrum of agricultural protection.

Methods can further comprise the use of plant growth regulators such as aviglycine, N-(phenylmethyl)-1H-purin-6-amine, ethephon, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

Methods include stringently and/or specifically controlling expression of a polynucleotide of interest. Stringency and/or specificity of modulating can be influenced by selecting the combination of elements used in the switch. These include, but are not limited to the promoter operably linked to the chemically-regulated transcriptional repressor, the chemically-regulated transcriptional repressor, the repressible promoter operably linked to the polynucleotide of interest, the polynucleotide of interest, the silencing element and the repressible promoter operably linked to the silencing element. Further control is provided by selection, dosage, conditions, and/or timing of the application of the chemical ligand. In some examples the expression of the polynucleotide of interest can be controlled more stringently, controlled in various tissues or cells, restricted to selected tissue or cell type, restricted to specific developmental stage(s), restricted to specific environmental conditions, and/or restricted to specific generation of a plant or progeny thereof. In some examples the repressor is operably linked to a constitutive promoter.

In some examples, the methods and compositions comprises a chemical-gene switch which may comprise additional elements. In some examples, one or more additional elements may provide means by which expression of the polynucleotide of interest can be controlled more stringently, controlled in various tissues or cells, restricted to selected tissue or cell type, restricted to specific developmental stage(s), restricted to specific environmental conditions, and/or restricted to specific generation of a plant or progeny thereof. In some examples those elements include site-specific recombination sites, site-specific recombinases, or combinations thereof.

In some methods, the chemical-gene switch may comprise a polynucleotide encoding a chemically-regulated transcriptional repressor, a promoter linked to a polynucleotide of interest comprising a sequence flanked by site-specific recombination sites, the silencing element operably linked to a repressible promoter, and a repressible promoter operably linked to a site-specific recombinase that specifically recognizes the site-specific recombination sites and implements a recombination event. In some examples, the recombination event is excision of the sequence flanked by the recombination sites. In some instances, the excision creates an operable linkage between the promoter and the polynucleotide of interest. In some examples, the promoter operably linked to the polynucleotide of interest is a non-constitutive promoter, including but not limited to a tissue preferred promoter, an inducible promoter, a repressible promoter, a developmental stage preferred promoter, or a promoter having more than one of these properties. In some examples expression of the polynucleotide of interest is primarily regulated in roots, leaves, stems, flowers, silks, anthers, pollen, meristem, germline, seed, endosperm, embryos, or progeny.

### VI. Novel Su Chemically-Regulated Transcriptional Regulators and Compositions and Methods Employing the Same

Further provided are methods and compositions which employ novel SU chemically-regulated transcriptional regulators. Non-limiting examples of these novel polynucleotides are set forth in SEQ ID NOS: 1193-1380 and 1949-2029 or active variants and fragments thereof and the encoded polypeptides set forth in SEQ ID NOS: 1381-1568 and 2030-2110 or active variants and fragments thereof.

Fragments and variants of SU chemically-regulated transcriptional regulators polynucleotides and polypeptides are also encompassed by the present invention. By “fragment” is intended a portion of the polynucleotide or a portion of the amino acid sequence and hence protein encoded thereby. Fragments of a polynucleotide may encode protein fragments that bind to a polynucleotide comprising an operator sequence, wherein the binding is regulated by a sulfonylurea compound. Alternatively, fragments of a polynucleotide that is useful as hybridization probes generally do not encode fragment proteins retaining biological activity. Thus, fragments of a nucleotide sequence may range from at least about 20 nucleotides, about 50 nucleotides, about 100 nucleotides, and up to the full-length polynucleotide encoding the SU chemically-regulated transcriptional regulators polypeptides.

A fragment of an SU chemically-regulated transcriptional regulators polynucleotide that encodes a biologically active portion of a SU chemically-regulated transcriptional regulator will encode at least 50, 75, 100, 150, 175, 200, 225, 250, 275, 300, 325, 350, 375, 400, 410, 415, 420, 425, 430, 435, or 440 contiguous amino acids, or up to the total number of amino acids present in a full-length SU chemically-regulated transcriptional regulators polypeptide. Fragments of an SU chemically-regulated transcriptional regulator polynucleotide that are useful as hybridization probes or PCR primers generally need not encode a biologically active portion of an SU chemically-regulated transcriptional regulator protein.

Thus, a fragment of an SU chemically-regulated transcriptional regulator polynucleotide may encode a biologically active portion of an SU chemically-regulated transcriptional regulator polypeptide, or it may be a fragment that can be used as a hybridization probe or PCR primer using methods disclosed below. A biologically active portion of an SU chemically-regulated transcriptional regulator polypeptide can be prepared by isolating a portion of one of the SU chemically-regulated transcriptional regulator polynucleotides, expressing the encoded portion of the SU chemically-regulated transcriptional regulator polypeptides (e.g., by recombinant expression in vitro), and assessing the activity of the portion of the SU chemically-regulated transcriptional regulator protein. Polynucleotides that are fragments of an SU chemically-regulated transcriptional regulator nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length SU chemically-regulated transcriptional regulator polynucleotide disclosed herein.

“Variant” protein is intended to mean a protein derived from the protein by deletion (i.e., truncation at the 5′ and/or 3′ end) and/or a deletion or addition of one or more amino acids at one or more internal sites in the native protein and/or substitution of one or more amino acids at one or more sites in the native protein. Variant proteins encompassed are biologically active, that is they continue to possess the desired biological activity of the native protein, that is, bind to a polynucleotide comprising an operator sequence, wherein the binding is regulated by a sulfonylurea compound. Such variants may result from, for example, genetic polymorphism or from human manipulation.

“Variants” is intended to mean substantially similar sequences. For polynucleotides, a variant comprises a polynucleotide having a deletion (i.e., truncations) at the 5′ and/or 3′ end and/or a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the SU chemically-regulated transcriptional regulator polypeptides. Naturally occurring variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis or gene synthesis but which still encode an SU chemically-regulated transcriptional regulator polypeptide.

Biologically active variants of an SU chemically-regulated transcriptional regulator polypeptide (and the polynucleotide encoding the same) will have at least about 75%, 80%, 85%, 90%, 91%, 92%, 93%, 94%, 95%, 96%, 97%, 98%, 99%, or more sequence identity to the polypeptide of any one of SEQ ID NO: 1381-1568 and 2030-2110 or with regard to any of the SU chemically-regulated transcriptional regulator polypeptides as determined by sequence alignment programs and parameters described elsewhere herein.

In still further embodiments, a biologically active variant of an SU chemically-regulated transcriptional regulator protein may differ from that protein by 200, 190, 180, 170, 160, 150, 140, 130, 120, 110, 100, 90, 80, 70, 60, 50, 40, 30, 25, 20, 19, 18, 17, 16 amino acid residues, as few as 1-15 amino acid residues, as few as 1-10, such as 6-10, as few as 10, 9, 8, 7, 6, 5, as few as 4, 3, 2, or even 1 amino acid residue.

The SU chemically-regulated transcriptional regulator polypeptide and the active variants and fragments thereof may be altered in various ways including amino acid substitutions, deletions, truncations, and insertions. Methods for such manipulations are generally known in the art. For example, amino acid sequence variants and fragments of the HPPD proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Obviously, the mutations that will be made in the DNA encoding the variant must not place the sequence out of reading frame and optimally will not create complementary regions that could produce secondary mRNA structure. See, EP Patent Application Publication No. 75,444.

Variant polynucleotides and proteins also encompass sequences and proteins derived from a mutagenic and recombinogenic procedure such as DNA shuffling. With such a procedure, one or more different SU chemically-regulated transcriptional regulator coding sequences can be manipulated to create a new SU chemically-regulated transcriptional regulator possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the SU chemically-regulated transcriptional regulator sequences disclosed herein and other known SU chemically-regulated transcriptional regulator genes to obtain a new gene coding for a protein with an improved property of interest. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Polynucleotides encoding the SU chemically-regulated transcriptional regulator polypeptide and the active variants and fragments thereof can be introduced into any of the DNA constructs discussed herein and further can be operably linked to any promoter sequence of interest. These constructs can be introduced/expressed in a host cell such as bacteria, yeast, insect, mammalian, or plant cells. Details for such methods are disclosed elsewherein herein, as is a detailed list of plants and plant cells that the sequences can be introduced into. Thus, various host cells, plants and plant cells are provided comprising the novel SU chemically-regulated transcriptional activators, including but not limited to, monocots and dicot plants such as corn, alfalfa, sunflower, Brassica, soybean, cotton, safflower, peanut, sorghum, wheat, millet, tobacco, etc.

In one embodiment, the novel SuR can be designed to contain a variety of different DNA binding domains and thereby bind a variety of different operators and influence transcription. In one embodiment, the SuR polypeptide comprises a DNA binding domain that specifically binds to a tetracycline operator. Thus, in specific embodiments, the SuR polypeptide or the polynucleotide encoding the same can comprise a DNA binding domain, including but not limited to, an operator DNA binding domain from repressors included tet, lac, trp, phd, arg, LexA, phiCh1 repressor, lambda C1 and Cro repressors, phage X repressor, MetJ, phir1t rro, phi434 C1 and Cro repressors, RafR, gal, ebg, uxuR, exuR, ROS, SinR, PurR, FruR, P22 C2, TetC, AcrR, Betl, Bm3R1, EnvR, QacR, MtrR, TcmR, Ttk, YbiH, YhgD, and mu Ner, or DNA binding domains in Interpro families including, but not limited to, IPR001647, IPR010982, and IPR01199, or an active variant or fragment thereof. Thus, the DNA binding specificity can be altered by fusing a SuR ligand binding domain to an alternate DNA binding domain. For example, the DNA binding domain from TetR class D can be fused to a SuR ligand binding domain to create SuR polypeptides that specifically bind to polynucleotides comprising a class D tetracycline operator. In some examples, a DNA binding domain variant or derivative can be used. For example, a DNA binding domain from a TetR variant that specifically recognizes a tetO-4C operator or a tetO-6C operator could be used (Helbl & Hillen (1998) J Mol Biol 276:313-318; Helbl et al. (1998) J Mol Biol 276:319-324).

In some examples, the chemically-regulated transcriptional repressor, or the polynucleotide encoding the same, includes a SuR polypeptide comprising a ligand binding domain comprising at least one amino acid substitution to a wild type tetracycline repressor protein ligand binding domain fused to a heterologous operator DNA binding domain which specifically binds to a polynucleotide comprising the operator sequence or derivative thereof, wherein repressor-operator binding is regulated by the absence or presence of a sulfonylurea compound. In specific embodiments, the heterologous operator DNA binding domain comprises a tetracycline operator sequence or active variant or fragment thereof, such that the repressor-operator binding is regulated by the absence or presence of a sulfonylurea compound.

In some examples, the SuR polypeptides, or polynucleotide encoding the same, comprise an amino acid substitution in the ligand binding domain of a wild type tetracycline repressor protein. In class B and D wild type TetR proteins, amino acid residues 6-52 represent the DNA binding domain. The remainder of the protein is involved in ligand binding and subsequent allosteric modification. For class B TetR residues 53-207 represent the ligand binding domain, while residues 53-218 comprise the ligand binding domain for the class D TetR. In some embodiments, the SuR polypeptides comprise at least one amino acid substitution in the ligand binding domain of a wild type TetR(B) protein, while in further examples, the SuR polypeptides comprise at least one amino acid substitution in the ligand binding domain of a wild type TetR(B) protein of SEQ ID NO:1.

In non-limiting embodiments, the SuR polypeptides can have an equilibrium binding constant for a sulfonylurea compound greater than 0.1 nM and less than 10 μM. In some examples, the SuR polypeptide has an equilibrium binding constant for a sulfonylurea compound of at least 0.1 nM, 0.5 nM, 1 nM, 10 nM, 50 nM, 100 nM, 250 nM, 500 nM, 750 nM, 1 μM, 5 μM, 7 μM but less than 10 μM. In other examples, the SuR polypeptide has an equilibrium binding constant for a sulfonylurea compound of at least 0.1 nM, 0.5 nM, 1 nM, 10 nM, 50 nM, 100 nM, 250 nM, 500 nM, 750 nM but less than 1 μM. In some embodiments, the SuR polypeptide has an equilibrium binding constant for a sulfonylurea compound greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples, the sulfonylurea compound is a chlorsulfuron, an ethametsulfuron, a metsulfuron, a sulfometuron, a tribenuron, a chlorimuron, a nicosulfuron, a rimsulfuron and/or a thifensulfuron. In further embodiments, the SuR as set forth in SEQ ID NOS: 1381-1568 and 2030-2110 has an equilibrium binding constant for chlorsulruon. In other embodiments, the SuR as set forth in SEQ ID NO: 1381-1568 and 2030-2110 has an equilibrium binding constant for ethametsulfuron.

In some examples, the SuR polypeptides have an equilibrium binding constant for an operator sequence greater than 0.1 nM and less than 10 μM. In some examples the SuR polypeptide has an equilibrium binding constant for an operator sequence of at least 0.1 nM, 0.5 nM, 1 nM, 10 nM, 50 nM, 100 nM, 250 nM, 500 nM, 750 nM, 1 μM, 5 μM, 7 μM but less than 10 μM. In some examples, the SuR polypeptide has an equilibrium binding constant for an operator sequence of at least 0.1 nM, 0.5 nM, 1 nM, 10 nM, 50 nM, 100 nM, 250 nM, 500 nM, 750 nM but less than 1 μM. In some examples the SuR polypeptide has an equilibrium binding constant for an operator sequence greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples, the operator sequence is a Tet operator sequence. In some examples, the Tet operator sequence is a TetR(A) operator sequence, a TetR(B) operator sequence, a TetR(D) operator sequence, TetR(E) operator sequence, a TetR(H) operator sequence, or a functional derivative thereof.

Various chemical ligands, including exemplary sulfonylurea chemical ligands, and the level and manner of application are discussed in detail elsewhere herein.

Various methods of employing Non-limiting examples of SuR polypeptides are set forth in U.S. Utility application Ser. No. 13/086,765, filed on Apr. 14, 2011 and in US Application Publication 2010-0105141, both of which are herein incorporated by reference in their entirety. Briefly, methods are further provided to regulate expression in a plant. The method comprises (a) providing a plant comprising (i) a first polynucleotide construct comprising a polynucleotide encoding a chemically-regulated transcriptional repressor operably linked to a promoter active in said plant, and, (ii) a second polynucleotide construct comprising a polynucleotide of interest operably linked to a first repressible promoter; wherein said first repressible promoter comprises at least one operator, wherein said chemically-regulated transcriptional repressor can bind to said operators in the absence of a chemical ligand and thereby repress transcription from said first repressible promoter in the absence of said chemical ligand, and wherein said plant is tolerant to said chemical ligand; (b) providing the plant with an effective amount of the chemical ligand whereby expression of said polynucleotide of interest are increased.

### VII. Sequence Identity

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

By “fragment” is intended a portion of the polynucleotide. fragments of a nucleotide sequence may range from at least about 10, about 15, 20 nucleotides, about 50 nucleotides, about 75 nucleotides, about 100 nucleotides, 200 nucleotides, 300 nucleotides, 400 nucleotides, 500 nucleotides, 600 nucleotides, 700 nucleotides and up to the full-length any polynucleotide of the chemical-gene switch system. Methods to assay for the activity of a desired polynucleotide or polypeptide are described elsewhere herein.

“Variants” is intended to mean substantially similar sequences. For polynucleotides or polypeptides, a variant comprises a deletion and/or addition of one or more nucleotides or amino acids at one or more internal sites within the native polynucleotide or polypeptide and/or a substitution of one or more nucleotides or amino acids at one or more sites in the original polynucleotide or original polypeptide. Generally, variants of a particular polynucleotide or polypeptide employed herein having the desired activity will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide or polypeptide as determined by sequence alignment programs and parameters described elsewhere herein.

An “isolated” or “purified” polynucleotide or polypeptide or biologically active fragment or variant thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated nucleic acid molecules can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

Non-limiting embodiments include:

1. A recombinant polynucleotide construct comprising:

(a) a polynucleotide of interest operably linked to a first repressible promoter active in a plant, wherein said first repressible promoter comprises at least one operator;

(b) a polynucleotide encoding a chemically-regulated transcriptional repressor operably linked to a promoter active in said plant; and

(c) a gene silencing construct operably linked to a second repressible promoter, wherein said gene silencing construct encodes a silencing element that decreases said chemically-regulated transcriptional repressor, wherein said second repressible promoter comprises at least one operator, and wherein said chemically-regulated transcriptional repressor can bind to each of said operators in the absence of a chemical ligand and thereby repress transcription from said first and said second repressible promoters in the absence of said chemical ligand.

2. The recombinant polynucleotide construct of embodiment 1, wherein

(i) said first repressible promoter operably linked to said polynucleotide of interest comprises three of said operators; and/or

(ii) said promoter operably linked to said polynucleotide encoding said chemically-regulated transcriptional repressor comprises a third repressible promoter, wherein said third repressible promoter comprises at least one operator; and/or

(iii) said second repressible promoter operably linked to said gene silencing construct comprises three of said operators.

3. The recombinant polynucleotide construct of embodiment 2, wherein said third repressible promoter operably linked to said polynucleotide encoding said chemically-regulated transcriptional repressor comprises two operators.

4. The recombinant polynucleotide construct of embodiment 2, wherein said third repressible promoter operably linked to said polynucleotide encoding said chemically-regulated transcriptional repressor comprises three operators.

5. The recombinant polynucleotide construct of any one of embodiments 1-4, wherein said polynucleotide encoding said chemically-regulated transcriptional repressor is regulated by a sulfonylurea compound.

6. The recombinant polynucleotide construct of embodiment 5, wherein said sulfonylurea compound comprises a pyrimidinylsulfonylurea, a triazinylsulfonylurea, a thiadazolylurea compound, a chlorosulfuron, an ethametsulfuron, a thifensulfuron, a metsulfuron, a sulfometuron, a tribenuron, a chlorimuron, a nicosulfuron, or a rimsulfuron compound.

7. The recombinant polynucleotide construct of any one of embodiments 1-4, wherein said polynucleotide encoding said chemically-regulated transcriptional repressor is regulated by tetracycline.

8. The recombinant polynucleotide construct of any one of embodiments 1-7, wherein said gene silencing construct encodes a cell non-autonomous silencing element that decreases said chemically-regulated transcriptional repressor.

9. The recombinant polynucleotide construct of any one of embodiments 1-7, wherein said silencing element comprises a siRNA, a trans-acting siRNA (TAS) or an amiRNA.

10. The recombinant polynucleotide construct of any one of embodiments 1-7, wherein said silencing element comprises a hairpin RNA.

11. The recombinant polynucleotide construct of embodiment 10, wherein said gene silencing construct comprising the silencing element comprises, in the following order, a first segment, a second segment, and a third segment, wherein
(a) said first segment comprises at least about 20 nucleotides having at least 90% sequence complementarity to the polynucleotide encoding said chemically-regulated transcriptional repressor;
(b) said second segment comprises a loop of sufficient length to allow the silencing element to be transcribed as a hairpin RNA; and,
(c) said third segment comprises at least about 20 nucleotides having at least 85% complementarity to the first segment.

12. A plant cell comprising

(a) a first polynucleotide construct comprising a polynucleotide of interest operably linked to a first repressible promoter active in said plant cell, wherein said first repressible promoter comprises at least one operator;

(b) a second polynucleotide construct comprising a polynucleotide encoding a chemically-regulated transcriptional repressor operably linked to a promoter active in said plant cell; and,

(c) a third polynucleotide construct comprising a gene silencing construct operably linked to a second repressible promoter comprising at least one operator,

wherein (i) said gene silencing construct encodes a cell non-autonomous silencing element that decreases the level of said chemically-regulated transcriptional repressor, (ii) said second repressible promoter comprises at least one operator regulating expression of the gene silencing construct, (iii) said chemically-regulated transcriptional repressor can bind to each of said operators in the absence of a chemical ligand and thereby repress transcription of said first and said second repressible promoters in the absence of said chemical ligand, and (iv) said plant cell is tolerant to the chemical ligand.

13. The plant cell of embodiment 12, wherein said first, second, and third polynucleotide constructs are contained on the same recombinant polynucleotide.

14. The plant cell of any one of embodiments 12-13, wherein

(i) said first repressible promoter operably linked to said polynucleotide of interest comprises three of said operators; and/or

(ii) said promoter operably linked to said polynucleotide encoding said chemically-regulated transcriptional repressor comprises a third repressible promoter, wherein said third repressible promoter comprises at least one operator regulating expression of said repressor; and/or

(iii) said second repressible promoter operably linked to said gene silencing construct comprises three of said operators.

15. The plant cell of embodiment 14, wherein said third repressible promoter operably linked to said polynucleotide encoding said chemically-regulated transcriptional repressor comprises two operators.

16. The plant cell of embodiment 14, wherein said third repressible promoter operably linked to said polynucleotide encoding said chemically-regulated transcriptional repressor comprises three operators.

17. The plant cell of any one of embodiments 12-16, wherein said chemically-regulated transcriptional repressor has a chemical ligand comprising a sulfonylurea compound.

18. The plant cell of embodiment 17, wherein said sulfonylurea compound comprises a pyrimidinylsulfonylurea, a triazinylsulfonylurea, a thiadazolylurea compound, a chlorosulfuron, an ethametsulfuron, a thifensulfuron, a metsulfuron, a sulfometuron, a tribenuron, a chlorimuron, a nicosulfuron, or a rimsulfuron compound.

19. The plant cell of any one of embodiments 12-16, wherein said chemically-regulated transcriptional repressor has a chemical ligand comprising tetracycline.

20. The plant cell of any one of embodiments 12-19, wherein said gene silencing construct encodes a cell non-autonomous silencing element that decreases said chemically-regulated transcriptional repressor.

21. The plant cell of any one of embodiments 12-19, wherein said silencing element comprises a siRNA, a trans-acting siRNA (TAS) or an amiRNA.

22. The plant cell of any one of embodiments 12-19, wherein said silencing element comprises a hairpin RNA.

23. The plant cell of embodiment 22, wherein said gene silencing construct comprising the silencing element comprises, in the following order, a first segment, a second segment, and a third segment, wherein

(a) said first segment comprises at least about 20 nucleotides having at least 90% sequence complementarity to the polynucleotide encoding said chemically-regulated transcriptional repressor;

(b) said second segment comprises a loop of sufficient length to allow the silencing element to be transcribed as a hairpin RNA; and,

(c) said third segment comprises at least about 20 nucleotides having at least 85% complementarity to the first segment.

24. A plant comprising the plant cell of any one of embodiments 12-23.

25. The plant of embodiment 24, wherein said plant is a monocot or dicot.

26. The plant of embodiment 25, wherein said plant is maize, barley, millet, wheat, rice, sorghum, rye, soybean, canola, alfalfa, sunflower, safflower, sugarcane, tobacco, Arabidopsis, or cotton.

27. The plant of any one of embodiments 24-26, wherein providing the plant with an effective amount of the chemical ligand (i) increases expression of said polynucleotide of interest and said silencing construct and (ii) decreases the level of said chemically-regulated transcriptional repressor in said plant or a part thereof.

28. The plant of embodiment 27, wherein providing an effective amount of said chemical ligand to said plant results in spatially or temporally extended expression of said polynucleotide of interest in said plant as compared to expression in a plant having been contacted with said effective amount of said chemical ligand and lacking said gene silencing construct.

29. The plant of embodiment 28, wherein said spatially or temporally extended expression of said polynucleotide of interest is achieved in said plant by providing an amount of chemical ligand smaller than the amount required to induce expression of said polynucleotide of interest in a plant lacking said gene silencing construct.

30. The plant of embodiment 28, wherein said spatially extended expression of said polynucleotide of interest comprises expression in at least one tissue of said plant not penetrated by the effective amount of said chemical ligand.

31. The plant of any one of embodiments 27-30, wherein providing said chemical ligand results in the complete penetration of expression of the polynucleotide of interest in the shoot apical meristem of said plant.

32. The plant of any one of embodiments 27-30, wherein providing said chemical ligand results in the complete penetration of expression of said polynucleotide of interest throughout the plant.

33. A transformed seed of the plant of any one of embodiments 25-32, wherein said seed comprises said first, second, and third polynucleotide construct.

34. The transformed seed of embodiment 33, wherein said first, second, and third polynucleotide constructs are contained on the same recombinant polynucleotide.

35. A method to regulate expression in a plant, comprising

(a) providing a plant comprising (i) a first polynucleotide construct comprising a polynucleotide encoding a chemically-regulated transcriptional repressor operably linked to a promoter active in said plant, (ii) a second polynucleotide construct comprising a polynucleotide of interest operably linked to a first repressible promoter, and (iii) a third polynucleotide construct comprising a gene silencing construct operably linked to a second repressible promoter,

wherein said gene silencing construct encodes a silencing element that decreases the level said chemically-regulated transcriptional repressor, wherein said first and second repressible promoters each comprise at least one operator, wherein said chemically-regulated transcriptional repressor can bind to each of said operators in the absence of a chemical ligand and thereby repress transcription from said first and said second repressible promoters in the absence of said chemical ligand, and wherein said plant is tolerant to said chemical ligand; and

(b) providing the plant with an effective amount of the chemical ligand whereby (i) expression of said polynucleotide of interest and said silencing construct are increased and (ii) the level of said chemically-regulated transcriptional repressor is decreased.

36. The method of embodiment 35, wherein providing an effective amount of said chemical ligand to said plant results in spatially or temporally extended expression of said polynucleotide of interest in said plant as compared to expression in a plant having been contacted with said effective amount of said chemical ligand and lacking said gene silencing construct.

37. The method of embodiment 36, wherein said spatially or temporally extended expression of said polynucleotide of interest is achieved by providing an amount of chemical ligand smaller than the amount required to induce expression of said polynucleotide of interest in a plant lacking said gene silencing construct.

38. The method of any one of embodiments 36-37, wherein said spatially extended expression of said polynucleotide of interest comprises expression in at least one tissue of said plant not penetrated by the effective amount of said chemical ligand.

39. The method of any one of embodiments 35-38, wherein providing said chemical ligand results in the spatially complete penetration of expression of the polynucleotide of interest in the shoot apical meristem of said plant.

40. The method of any one of embodiments 35-38, wherein providing said chemical ligand results in the complete penetration of expression of said polynucleotide of interest throughout the plant.

41. The method of any one of embodiments 35-40, wherein said chemical ligand is provided by spraying.

42. The method of any one of embodiments 35-40, wherein said chemical ligand is provided by seed treatment.

43. The method of any one of embodiments 35-42, wherein said first repressible promoter operably linked to said polynucleotide of interest comprises three of said operators, wherein said promoter operably linked to said chemically-regulated transcriptional repressor comprises a third repressible promoter, wherein said third repressible promoter comprises at least one operator, and wherein said second repressible promoter operably linked to said gene silencing construct comprises three of said operators.

44. The method of embodiment 43, wherein said third repressible promoter operably linked to said chemically-regulated transcriptional repressor comprises two operators.

45. The method of embodiment 43, wherein said third repressible promoter operably linked to said chemically-regulated transcriptional repressor comprises three operators.

46. The method of any one of embodiments 35-45, wherein expression of the polynucleotide of interest alters the phenotype of the plant.

47. The method of any one of embodiments 35-45, wherein expression of the polynucleotide of interest alters the genotype of the plant.

48. The method of any one of embodiments 35-47, wherein said chemically-regulated transcriptional repressor has a chemical ligand comprising a sulfonylurea compound.

49. The method of embodiment 48, wherein said sulfonylurea compound comprises a pyrimidinylsulfonylurea, a triazinylsulfonylurea, a thiadazolylurea, a chlorosulfuron, an ethametsulfuron, a thifensulfuron, a metsulfuron, a sulfometuron, a tribenuron, a chlorimuron, a nicosulfuron, or a rimsulfuron compound.

50. The method of any one of embodiments 35-47, wherein said chemically-regulated transcriptional repressor has a chemical ligand comprising tetracycline.

51. The method of any one of embodiments 35-47, wherein said gene silencing construct encodes a cell non-autonomous silencing element that decreases said chemically-regulated transcriptional repressor.

52. The method of any one of embodiments 35-47, wherein said silencing element comprises a siRNA, a trans-acting siRNA (TAS) or an amiRNA.

53. The method of any one of embodiments 35-47, wherein said silencing element comprises a hairpin RNA.

54. The method of embodiment 53, wherein said gene silencing construct comprising the silencing element comprises, in the following order, a first segment, a second segment, and a third segment, wherein

(a) said first segment comprises at least about 20 nucleotides having at least 90% sequence complementarity to said chemically-regulated transcriptional repressor;

(b) said second segment comprises a loop of sufficient length to allow the silencing element to be transcribed as a hairpin RNA; and

(c) said third segment comprises at least about 20 nucleotides having at least 85% complementarity to the first segment.

55. The method of any one of embodiments 35-54, wherein said silencing element is transported by the vasculature of said plant.

TABLE 1A
Non-limiting examples of components of the chemical-
gene switch presented in the sequence listing.

| SEQ ID NO | Brief Description |
|---|---|
| 1 | Amino acid sequence of TetR(B) |
| 2 | Amino acid sequence of a variant of SEQ ID NO: 1 |
| 3-13 | Amino acid sequence for some Su(R) polypeptides |
| 14-204 | Amino acid sequence for Su(R) polypeptides that |
| | can employ ethametsulfuron as a chemical ligand |
| 205-419 | Amino acid sequence for Su(R) polypeptides that |
| | can employ chlorsulfuron a chemical ligand. |
| 412-419 | Amino acid sequence of Su(R) polypeptides with reverse |
| | repressor activity |
| 420-430 | Nucleic acid sequence encoding SEQ ID NO: 3-13 |
| 431-621 | Nucleic acid sequence encoding SEQ ID NO: 431-621 |
| 622-836 | Nucleic acid sequence encoding SEQ ID NO: 405-419 |
| 837-840 | oligonucleotides |
| 841-847 | Various constructs |
| 848 | Tet operator sequence |
| 849 | Plant actin promoter |
| 850 | banana streak virus promoter (BSV) |
| 851 | a mirabilis mosaic virus promoter |
| 852 | enhanced MMV promoter (dMMV) |
| 853 | plant P450 promoter (MP1) |
| 854 | elongation factor 1a (EF1A) promoter |
| 855 | Plant actin promoter with tet op (actin/Op) |
| 856 | Banana steak virus promoter with tet op (BSV/Op) |
| 857 | mirabilis mosaic virus promoter with tet op (MMV/Op) |
| 858 | enhanced MMV promoter with tet op (dMMV/Op) |
| 859 | plant P450 promoter with tet op (MP1/Op) |
| 860 | elongation factor 1a promoter with tet op (EF1A/Op) |
| 861 | 35S CaMV promoter with ADH1 intron |
| 862 | 35SCaMV promoter engineered with tet operators |

TABLE 1B
Non-limiting examples of components of the chemical-
gene switch presented in the sequence listing.

| | | Description/clone |
|---|---|---|
| SEQ ID NO | type | name |
| 3 | AA | L1-02 |
| 4 | AA | L1-07 |
| 5 | AA | L1-09 |
| 6 | AA | L1-20 |
| 7 | AA | L1-22 |
| 8 | AA | L1-24 |
| 9 | AA | L1-28 |
| 10 | AA | L1-29 |
| 11 | AA | L1-31 |
| 12 | AA | L1-38 |
| 13 | AA | L1-44 |
| 14 | AA | L6-1B03 |
| 15 | AA | L6-1C03 |
| 16 | AA | L6-1C06 |
| 17 | AA | L6-1G06 |
| 18 | AA | L6-1G07 |
| 19 | AA | L6-1G09 |
| 20 | AA | L6-1G10 |
| 21 | AA | L6-1G11 |
| 22 | AA | L6-1H12 |
| 23 | AA | L6-2A01 |
| 24 | AA | L6-2A02 |
| 25 | AA | L6-2A04 |
| 26 | AA | L6-2A06 |
| 27 | AA | L6-2A12 |
| 28 | AA | L6-2B04 |
| 29 | AA | L6-2B06 |
| 30 | AA | L6-2B08 |
| 31 | AA | L6-2B09 |
| 32 | AA | L6-2B10 |
| 33 | AA | L6-2B11 |
| 34 | AA | L6-2C02 |
| 35 | AA | L6-2C05 |
| 36 | AA | L6-2C09 |
| 37 | AA | L6-2C10 |
| 38 | AA | L6-2C11 |
| 39 | AA | L6-2D01 |
| 40 | AA | L6-2D02 |
| 41 | AA | L6-2D03 |
| 42 | AA | L6-2D04 |
| 43 | AA | L6-2D07 |
| 44 | AA | L6-2D11 |
| 45 | AA | L6-2D12 |
| 46 | AA | L6-2E02 |
| 47 | AA | L6-2E03 |
| 48 | AA | L6-2E04 |
| 49 | AA | L6-2E05 |
| 50 | AA | L6-2E07 |
| 51 | AA | L6-2E08 |
| 52 | AA | L6-2E09 |
| 53 | AA | L6-2E11 |
| 54 | AA | L6-2F08 |
| 55 | AA | L6-2F10 |
| 56 | AA | L6-2F11 |
| 57 | AA | L6-2F12 |
| 58 | AA | L6-2G01 |
| 59 | AA | L6-2G02 |
| 60 | AA | L6-2G03 |
| 61 | AA | L6-2G05 |
| 62 | AA | L6-2G10 |
| 63 | AA | L6-2H01 |
| 64 | AA | L6-2H02 |
| 65 | AA | L6-2H03 |
| 66 | AA | L6-2H04 |
| 67 | AA | L6-2H06 |
| 68 | AA | L6-2H07 |
| 69 | AA | L6-2H10 |
| 70 | AA | L6-2H11 |
| 71 | AA | L6-3A01 |
| 72 | AA | L6-3A02 |
| 73 | AA | L6-3A03 |
| 74 | AA | L6-3A06 |
| 75 | AA | L6-3A11 |
| 76 | AA | L6-3B08 |
| 77 | AA | L6-3B09 |
| 78 | AA | L6-3C02 |
| 79 | AA | L6-3C04 |
| 80 | AA | L6-3C05 |
| 81 | AA | L6-3C06 |
| 82 | AA | L6-3D03 |
| 83 | AA | L6-3D05 |
| 84 | AA | L6-3D09 |
| 85 | AA | L6-3E08 |
| 86 | AA | L6-3E09 |
| 87 | AA | L6-3E10 |
| 88 | AA | L6-3F02 |
| 89 | AA | L6-3F09 |
| 90 | AA | L6-3F12 |
| 91 | AA | L6-3G03 |
| 92 | AA | L6-3G05 |
| 93 | AA | L6-3G09 |
| 94 | AA | L6-3H02 |
| 95 | AA | L6-3H05 |
| 96 | AA | L6-3H08 |
| 97 | AA | L6-4A01 |
| 98 | AA | L6-4A03 |
| 99 | AA | L6-4A04 |
| 100 | AA | L6-4A09 |
| 101 | AA | L6-4A10 |
| 102 | AA | L6-4A11 |
| 103 | AA | L6-4B05 |
| 104 | AA | L6-4B06 |
| 105 | AA | L6-4B07 |
| 106 | AA | L6-4B08 |
| 107 | AA | L6-4B12 |
| 108 | AA | L6-4C01 |
| 109 | AA | L6-4C03 |
| 110 | AA | L6-4C04 |
| 111 | AA | L6-4C07 |
| 112 | AA | L6-4C08 |
| 113 | AA | L6-4C09 |
| 114 | AA | L6-4C10 |
| 115 | AA | L6-4C11 |
| 116 | AA | L6-4D09 |
| 117 | AA | L6-4D10 |
| 118 | AA | L6-4E01 |
| 119 | AA | L6-4E02 |
| 120 | AA | L6-4E03 |
| 121 | AA | L6-4E05 |
| 122 | AA | L6-4E08 |
| 123 | AA | L6-4E09 |
| 124 | AA | L6-4E11 |
| 125 | AA | L6-4E12 |
| 126 | AA | L6-4F01 |
| 127 | AA | L6-4F10 |
| 128 | AA | L6-4F12 |
| 129 | AA | L6-4G02 |
| 130 | AA | L6-4G03 |
| 131 | AA | L6-4G06 |
| 132 | AA | L6-4G07 |
| 133 | AA | L6-4G08 |
| 134 | AA | L6-4G10 |
| 135 | AA | L6-4H07 |
| 136 | AA | L6-5A02 |
| 137 | AA | L6-5A03 |
| 138 | AA | L6-5A04 |
| 139 | AA | L6-5A05 |
| 140 | AA | L6-5A06 |
| 141 | AA | L6-5A07 |
| 142 | AA | L6-5A09 |
| 143 | AA | L6-5A10 |
| 144 | AA | L6-5B02 |
| 145 | AA | L6-5B07 |
| 146 | AA | L6-5B08 |
| 147 | AA | L6-5B11 |
| 148 | AA | L6-5C01 |
| 149 | AA | L6-5C02 |
| 150 | AA | L6-5C04 |
| 151 | AA | L6-5C08 |
| 152 | AA | L6-5C10 |
| 153 | AA | L6-5C11 |
| 154 | AA | L6-5D04 |
| 155 | AA | L6-5D09 |
| 156 | AA | L6-5D11 |
| 157 | AA | L6-5D12 |
| 158 | AA | L6-5E05 |
| 159 | AA | L6-5E09 |
| 160 | AA | L6-5F02 |
| 161 | AA | L6-5F04 |
| 162 | AA | L6-5F05 |
| 163 | AA | L6-5F07 |
| 164 | AA | L6-5F08 |
| 165 | AA | L6-5F10 |
| 166 | AA | L6-5F12 |
| 167 | AA | L6-5G03 |
| 168 | AA | L6-5G05 |
| 169 | AA | L6-5G06 |
| 170 | AA | L6-5G08 |
| 171 | AA | L6-5G11 |
| 172 | AA | L6-5G12 |
| 173 | AA | L6-5H03 |
| 174 | AA | L6-5H06 |
| 175 | AA | L6-5H07 |
| 176 | AA | L6-5H12 |
| 177 | AA | L6-6A09 |
| 178 | AA | L6-6B01 |
| 179 | AA | L6-6B03 |
| 180 | AA | L6-6B04 |
| 181 | AA | L6-6B05 |
| 182 | AA | L6-6B10 |
| 183 | AA | L6-6C01 |
| 184 | AA | L6-6C02 |
| 185 | AA | L6-6C04 |
| 186 | AA | L6-6C05 |
| 187 | AA | L6-6C06 |
| 188 | AA | L6-6C07 |
| 189 | AA | L6-6C10 |
| 190 | AA | L6-6C11 |
| 191 | AA | L6-6D02 |
| 192 | AA | L6-6D06 |
| 193 | AA | L6-6D07 |
| 194 | AA | L6-6D09 |
| 195 | AA | L6-6D10 |
| 196 | AA | L6-6D12 |
| 197 | AA | L6-6E01 |
| 198 | AA | L6-6E02 |
| 199 | AA | L6-6E03 |
| 200 | AA | L6-6E11 |
| 201 | AA | L6-6F03 |
| 202 | AA | L6-6F07 |
| 203 | AA | L6-6F08 |
| 204 | AA | L6-6G01 |
| 205 | AA | L7-1A01 |
| 206 | AA | L7-1B01 |
| 207 | AA | L7-1C01 |
| 208 | AA | L7-1D01 |
| 209 | AA | L7-1E01 |
| 210 | AA | L7-1F01 |
| 211 | AA | L7-1G01 |
| 212 | AA | L7-1C02 |
| 213 | AA | L7-1D02 |
| 214 | AA | L7-1E02 |
| 215 | AA | L7-1F02 |
| 216 | AA | L7-1G02 |
| 217 | AA | L7-1H02 |
| 218 | AA | L7-1C03 |
| 219 | AA | L7-1E03 |
| 220 | AA | L7-1A04 |
| 221 | AA | L7-1C04 |
| 222 | AA | L7-1D04 |
| 223 | AA | L7-1E04 |
| 224 | AA | L7-1F04 |
| 225 | AA | L7-1G04 |
| 226 | AA | L7-1H04 |
| 227 | AA | L7-1A05 |
| 228 | AA | L7-1C05 |
| 229 | AA | L7-1E05 |
| 230 | AA | L7-1F05 |
| 231 | AA | L7-1A06 |
| 232 | AA | L7-1B06 |
| 233 | AA | L7-1D06 |
| 234 | AA | L7-1E06 |
| 235 | AA | L7-1F06 |
| 236 | AA | L7-1G06 |
| 237 | AA | L7-1H06 |
| 238 | AA | L7-1A07 |
| 239 | AA | L7-1B07 |
| 240 | AA | L7-1C07 |
| 241 | AA | L7-1D07 |
| 242 | AA | L7-1E07 |
| 243 | AA | L7-1F07 |
| 244 | AA | L7-1G07 |
| 245 | AA | L7-1A08 |
| 246 | AA | L7-1C08 |
| 247 | AA | L7-1D08 |
| 248 | AA | L7-1E08 |
| 249 | AA | L7-1F08 |
| 250 | AA | L7-1G08 |
| 251 | AA | L7-1A09 |
| 252 | AA | L7-1B09 |
| 253 | AA | L7-1C09 |
| 254 | AA | L7-1D09 |
| 255 | AA | L7-1E09 |
| 256 | AA | L7-1G09 |
| 257 | AA | L7-1A10 |
| 258 | AA | L7-1B10 |
| 259 | AA | L7-1C10 |
| 260 | AA | L7-1D10 |
| 261 | AA | L7-1F10 |
| 262 | AA | L7-1A11 |
| 263 | AA | L7-1B11 |
| 264 | AA | L7-1C11 |
| 265 | AA | L7-1E11 |
| 266 | AA | L7-1A12 |
| 267 | AA | L7-1C12 |
| 268 | AA | L7-1F12 |
| 269 | AA | L7-1G12 |
| 270 | AA | L7-2A01 |
| 271 | AA | L7-2B01 |
| 272 | AA | L7-2D01 |
| 273 | AA | L7-2E01 |
| 274 | AA | L7-2F01 |
| 275 | AA | L7-2G01 |
| 276 | AA | L7-2H01 |
| 277 | AA | L7-2B02 |
| 278 | AA | L7-2D02 |
| 279 | AA | L7-2E02 |
| 280 | AA | L7-2F02 |
| 281 | AA | L7-2G02 |
| 282 | AA | L7-2H02 |
| 283 | AA | L7-2D03 |
| 284 | AA | L7-2E03 |
| 285 | AA | L7-2F03 |
| 286 | AA | L7-2G03 |
| 287 | AA | L7-2H03 |
| 288 | AA | L7-2D04 |
| 289 | AA | L7-2E04 |
| 290 | AA | L7-2F04 |
| 291 | AA | L7-2H04 |
| 292 | AA | L7-2B05 |
| 293 | AA | L7-2D05 |
| 294 | AA | L7-2E05 |
| 295 | AA | L7-2F05 |
| 296 | AA | L7-2H05 |
| 297 | AA | L7-2A06 |
| 298 | AA | L7-2C06 |
| 299 | AA | L7-2D06 |
| 300 | AA | L7-2F06 |
| 301 | AA | L7-2G06 |
| 302 | AA | L7-2A07 |
| 303 | AA | L7-2B07 |
| 304 | AA | L7-2C07 |
| 305 | AA | L7-2D07 |
| 306 | AA | L7-2E07 |
| 307 | AA | L7-2G07 |
| 308 | AA | L7-2B08 |
| 309 | AA | L7-2D08 |
| 310 | AA | L7-2F08 |
| 311 | AA | L7-2G08 |
| 312 | AA | L7-2B09 |
| 313 | AA | L7-2C09 |
| 314 | AA | L7-2E09 |
| 315 | AA | L7-2B10 |
| 316 | AA | L7-2E10 |
| 317 | AA | L7-2G10 |
| 318 | AA | L7-2C11 |
| 319 | AA | L7-2D11 |
| 320 | AA | L7-2F11 |
| 321 | AA | L7-2G11 |
| 322 | AA | L7-2B12 |
| 323 | AA | L7-2C12 |
| 324 | AA | L7-2D12 |
| 325 | AA | L7-2F12 |
| 326 | AA | L7-2G12 |
| 327 | AA | L7-3A01 |
| 328 | AA | L7-3C01 |
| 329 | AA | L7-3G01 |
| 330 | AA | L7-3H01 |
| 331 | AA | L7-3A02 |
| 332 | AA | L7-3B02 |
| 333 | AA | L7-3D02 |
| 334 | AA | L7-3G02 |
| 335 | AA | L7-3H02 |
| 336 | AA | L7-3B03 |
| 337 | AA | L7-3C03 |
| 338 | AA | L7-3E03 |
| 339 | AA | L7-3G03 |
| 340 | AA | L7-3H03 |
| 341 | AA | L7-3B04 |
| 342 | AA | L7-3E04 |
| 343 | AA | L7-3G04 |
| 344 | AA | L7-3A05 |
| 345 | AA | L7-3B05 |
| 346 | AA | L7-3H05 |
| 347 | AA | L7-3B06 |
| 348 | AA | L7-3D06 |
| 349 | AA | L7-3E06 |
| 350 | AA | L7-3A07 |
| 351 | AA | L7-3C07 |
| 352 | AA | L7-3F07 |
| 353 | AA | L7-3A08 |
| 354 | AA | L7-3B08 |
| 355 | AA | L7-3C08 |
| 356 | AA | L7-3F08 |
| 357 | AA | L7-3G08 |
| 358 | AA | L7-3B09 |
| 359 | AA | L7-3F09 |
| 360 | AA | L7-3A10 |
| 361 | AA | L7-3B10 |
| 362 | AA | L7-3C10 |
| 363 | AA | L7-3G10 |
| 364 | AA | L7-3A11 |
| 365 | AA | L7-3C11 |
| 366 | AA | L7-3E11 |
| 367 | AA | L7-3G11 |
| 368 | AA | L7-3A12 |
| 369 | AA | L7-3B12 |
| 370 | AA | L7-3C12 |
| 371 | AA | L7-3E12 |
| 372 | AA | L7-3F12 |
| 373 | AA | L7-3G12 |
| 374 | AA | L7-4A01 |
| 375 | AA | L7-4A03 |
| 376 | AA | L7-4A04 |
| 377 | AA | L7-4A06 |
| 378 | AA | L7-4A08 |
| 379 | AA | L7-4A09 |
| 380 | AA | L7-4A12 |
| 381 | AA | L7-4B03 |
| 382 | AA | L7-4B04 |
| 383 | AA | L7-4B06 |
| 384 | AA | L7-4B07 |
| 385 | AA | L7-4C01 |
| 386 | AA | L7-4C03 |
| 387 | AA | L7-4C04 |
| 388 | AA | L7-4C06 |
| 389 | AA | L7-4C09 |
| 390 | AA | L7-4C12 |
| 391 | AA | L7-4D04 |
| 392 | AA | L7-4D07 |
| 393 | AA | L7-4D08 |
| 394 | AA | L7-4D10 |
| 395 | AA | L7-4D11 |
| 396 | AA | L7-4E01 |
| 397 | AA | L7-4E02 |
| 398 | AA | L7-4E04 |
| 399 | AA | L7-4E05 |
| 400 | AA | L7-4E07 |
| 401 | AA | L7-4E08 |
| 402 | AA | L6-3A09 |
| 403 | AA | L7-4C06 |
| 404 | AA | L10-84 |
| 405 | AA | L13-2-46 |
| 406 | AA | L12-1-10 |
| 407 | AA | L13-2-23 |
| 408 | AA | L7-1C3-A5 |
| 409 | AA | L7-1F8-A11 |
| 410 | AA | L7-1G6-B2 |
| 411 | AA | L7-3E3-D1 |
| 412 | AA | L1-18 |
| 413 | AA | L1-21 |
| 414 | AA | L1-25 |
| 415 | AA | L1-33 |
| 416 | AA | L1-34 |
| 417 | AA | L1-36 |
| 418 | AA | L1-39 |
| 419 | AA | L1-41 |
| 420 | DNA | L1-02 CDS |
| 421 | DNA | L1-07 CDS |
| 422 | DNA | L1-09 CDS |
| 423 | DNA | L1-20 CDS |
| 424 | DNA | L1-22 CDS |
| 425 | DNA | L1-24 CDS |
| 426 | DNA | L1-28 CDS |
| 427 | DNA | L1-29 CDS |
| 428 | DNA | L1-31 CDS |
| 429 | DNA | L1-38 CDS |
| 430 | DNA | L1-44 CDS |
| 431 | DNA | L6-1B03 CDS |
| 432 | DNA | L6-1C03 CDS |
| 433 | DNA | L6-1C06 CDS |
| 434 | DNA | L6-1G06 CDS |
| 435 | DNA | L6-1G07 CDS |
| 436 | DNA | L6-1G09 CDS |
| 437 | DNA | L6-1G10 CDS |
| 438 | DNA | L6-1G11 CDS |
| 439 | DNA | L6-1H12 CDS |
| 440 | DNA | L6-2A01 CDS |
| 441 | DNA | L6-2A02 CDS |
| 442 | DNA | L6-2A04 CDS |
| 443 | DNA | L6-2A06 CDS |
| 444 | DNA | L6-2A12 CDS |
| 445 | DNA | L6-2B04 CDS |
| 446 | DNA | L6-2B06 CDS |
| 447 | DNA | L6-2B08 CDS |
| 448 | DNA | L6-2B09 CDS |
| 449 | DNA | L6-2B10 CDS |
| 450 | DNA | L6-2B11 CDS |
| 451 | DNA | L6-2C02 CDS |
| 452 | DNA | L6-2C05 CDS |
| 453 | DNA | L6-2C09 CDS |
| 454 | DNA | L6-2C10 CDS |
| 455 | DNA | L6-2C11 CDS |
| 456 | DNA | L6-2D01 CDS |
| 457 | DNA | L6-2D02 CDS |
| 458 | DNA | L6-2D03 CDS |
| 459 | DNA | L6-2D04 CDS |
| 460 | DNA | L6-2D07 CDS |
| 461 | DNA | L6-2D11 CDS |
| 462 | DNA | L6-2D12 CDS |
| 463 | DNA | L6-2E02 CDS |
| 464 | DNA | L62E03 CDS |
| 465 | DNA | L6-2E04 CDs |
| 466 | DNA | L6-2E05 CDS |
| 467 | DNA | L6-2E07 CDS |
| 468 | DNA | L6-2E08 CDS |
| 469 | DNA | L6-2E09 CDS |
| 470 | DNA | L6-2E11 CDS |
| 471 | DNA | L6-2F08 CDS |
| 472 | DNA | L6-2F10 CDS |
| 473 | DNA | L6-2F11 CDS |
| 474 | DNA | L6-2F12 CDS |
| 475 | DNA | L6-2G01 CDS |
| 476 | DNA | L6-2G02 CDS |
| 477 | DNA | L6-2G03 CDS |
| 478 | DNA | L6-2G05 CDS |
| 479 | DNA | L6-2G10 CDS |
| 480 | DNA | L6-2H01 CDS |
| 481 | DNA | L6-2H02 CDS |
| 482 | DNA | L6-2H03 CDS |
| 483 | DNA | L6-2H04 CDS |
| 484 | DNA | L6-2H06 CDS |
| 485 | DNA | L6-2H07 CDS |
| 486 | DNA | L6-2H10 CDS |
| 487 | DNA | L6-2H11 CDS |
| 488 | DNA | L6-3A01 CDS |
| 489 | DNA | L6-3A02 CDS |
| 490 | DNA | L6-3A03 CDS |
| 491 | DNA | L6-3A06 CDS |
| 492 | DNA | L6-3A11 CDS |
| 493 | DNA | L6-3B08 CDS |
| 494 | DNA | L6-3B09 CDS |
| 495 | DNA | L6-3C02 CDS |
| 496 | DNA | L6-3C04 CDS |
| 497 | DNA | L6-3C05 CDS |
| 498 | DNA | L6-3C06 CDS |
| 499 | DNA | L6-3D03 CDS |
| 500 | DNA | L6-3D05 CDS |
| 501 | DNA | L6-3D09 CDS |
| 502 | DNA | L6-3E08 CDS |
| 503 | DNA | L6-3E09 CDS |
| 504 | DNA | L6-3E10 CDS |
| 505 | DNA | L6-3F02 CDS |
| 506 | DNA | L6-3F09 CDS |
| 507 | DNA | L6-3F12 CDS |
| 508 | DNA | L6-3G03 CDS |
| 509 | DNA | L6-3G05 CDS |
| 510 | DNA | L6-3G09 CDS |
| 511 | DNA | L6-3H02 CDS |
| 512 | DNA | L6-3H05 CDS |
| 513 | DNA | L6-3H08 CDS |
| 514 | DNA | L6-4A01 CDS |
| 515 | DNA | L6-4A03 CDS |
| 516 | DNA | L6-4A04 CDS |
| 517 | DNA | L6-4A09 CDS |
| 518 | DNA | L6-4A10 CDS |
| 519 | DNA | L6-4A11 CDS |
| 520 | DNA | L6-4B05 CDS |
| 521 | DNA | L6-4B06 CDS |
| 522 | DNA | L6-4B07 CDS |
| 523 | DNA | L6-4B08 CDS |
| 524 | DNA | L6-4B12 CDS |
| 525 | DNA | L6-4C01 CDS |
| 526 | DNA | L6-4C03 CDS |
| 527 | DNA | L6-4C04 CDS |
| 528 | DNA | L6-4C07 CDS |
| 529 | DNA | L6-4C08 CDS |
| 530 | DNA | L6-4C09 CDS |
| 531 | DNA | L6-4C10 CDS |
| 532 | DNA | L6-4C11 CDS |
| 533 | DNA | L6-4D09 CDS |
| 534 | DNA | L6-4D10 CDS |
| 535 | DNA | L6-4E01 CDS |
| 536 | DNA | L6-4E02 CDS |
| 537 | DNA | L6-4E03 CDS |
| 538 | DNA | L6-4E05 CDS |
| 539 | DNA | L6-4E08 CDS |
| 540 | DNA | L6-4E09 CDS |
| 541 | DNA | L6-4E11 CDS |
| 542 | DNA | L6-4E12 CDS |
| 543 | DNA | L6-4F01 CDS |
| 544 | DNA | L6-4F10 CDS |
| 545 | DNA | L6-4F12 CDS |
| 546 | DNA | L6-4G02 CDS |
| 547 | DNA | L6-4G03 CDS |
| 548 | DNA | L6-4G06 CDS |
| 549 | DNA | L6-4G07 CDS |
| 550 | DNA | L6-4G08 CDS |
| 551 | DNA | L6-4G10 CDS |
| 552 | DNA | L6-4H07 CDS |
| 553 | DNA | L6-5A02 CDS |
| 554 | DNA | L6-5A03 CDS |
| 555 | DNA | L6-5A04 CDS |
| 556 | DNA | L6-5A05 CDS |
| 557 | DNA | L6-5A06 CDS |
| 558 | DNA | L6-5A07 CDS |
| 559 | DNA | L6-5A09 CDS |
| 560 | DNA | L6-5A10 CDS |
| 561 | DNA | L6-5B02 CDS |
| 562 | DNA | L6-5B07 CDS |
| 563 | DNA | L6-5B08 CDS |
| 564 | DNA | L6-5B11 CDS |
| 565 | DNA | L6-5C01 CDS |
| 566 | DNA | L6-5C02 CDS |
| 567 | DNA | L6-5C04 CDS |
| 568 | DNA | L6-5C08 CDS |
| 569 | DNA | L6-5C10 CDS |
| 570 | DNA | L6-5C11 CDS |
| 571 | DNA | L6-5D04 CDS |
| 572 | DNA | L6-5D09 CDS |
| 573 | DNA | L6-5D11 CDS |
| 574 | DNA | L6-5D12 CDS |
| 575 | DNA | L6-5E05 CDS |
| 576 | DNA | L6-5E09 CDS |
| 577 | DNA | L6-5F02 CDS |
| 578 | DNA | L6-5F04 CDS |
| 579 | DNA | L6-5F05 CDS |
| 580 | DNA | L6-5F07 CDS |
| 581 | DNA | L6-5F08 CDS |
| 582 | DNA | L6-5F10 CDS |
| 583 | DNA | L6-5F12 CDS |
| 584 | DNA | L6-5G03 CDS |
| 585 | DNA | L6-5G05 CDS |
| 586 | DNA | L6-5G06 CDS |
| 587 | DNA | L6-5G08 CDS |
| 588 | DNA | L6-5G11 CDS |
| 589 | DNA | L6-5G12 CDS |
| 590 | DNA | L6-5H03 CDS |
| 591 | DNA | L6-5H06 CDS |
| 592 | DNA | L6-5H07 CDS |
| 593 | DNA | L6-5H12 CDS |
| 594 | DNA | L6-6A09 CDS |
| 595 | DNA | L6-6B01 CDS |
| 596 | DNA | L6-6B03 CDS |
| 597 | DNA | L6-6B04 CDS |
| 598 | DNA | L6-6B05 CDS |
| 599 | DNA | L6-6B10 CDS |
| 600 | DNA | L6-6C01 CDS |
| 601 | DNA | L6-6C02 CDS |
| 602 | DNA | L6-6C04 CDS |
| 603 | DNA | L6-6C05 CDS |
| 604 | DNA | L6-6C06 CDS |
| 605 | DNA | L6-6C07 CDS |
| 606 | DNA | L6-6C10 CDS |
| 607 | DNA | L6-6C11 CDS |
| 608 | DNA | L6-6D02 CDS |
| 609 | DNA | L6-6D06 CDS |
| 610 | DNA | L6-6D07 CDS |
| 611 | DNA | L6-6D09 CDS |
| 612 | DNA | L6-6D10 CDS |
| 613 | DNA | L6-6D12 CDS |
| 614 | DNA | L6-6E01 CDS |
| 615 | DNA | L6-6E02 CDS |
| 616 | DNA | L6-6E03 CDS |
| 617 | DNA | L6-6E11CDS |
| 618 | DNA | L6-6F03 CDS |
| 619 | DNA | L6-6F07 CDS |
| 620 | DNA | L6-6F08 CDS |
| 621 | DNA | L6-6G01 CDS |
| 622 | DNA | L7-1A01 CDS |
| 623 | DNA | L7-1B01 Cds |
| 624 | DNA | L7-1C01 CDS |
| 625 | DNA | L7-1D01 CDS |
| 626 | DNA | L7-1E01 CDS |
| 627 | DNA | L7-1F01 CDS |
| 628 | DNA | L7-1G01 CDS |
| 629 | DNA | L7-1C02 CDS |
| 630 | DNA | L7-1D02 CDS |
| 631 | DNA | L7-1E02 CDS |
| 632 | DNA | L7-1F02 CDS |
| 633 | DNA | L7-1G02 CDS |
| 634 | DNA | L7-1H02 CDS |
| 635 | DNA | L7-1C03 CDS |
| 636 | DNA | L7-1E03 CDS |
| 637 | DNA | L7-1A04 CDS |
| 638 | DNA | L7-1C04 CDS |
| 639 | DNA | L7-1D04 CDS |
| 640 | DNA | L7-1E04 CDS |
| 641 | DNA | L7-1F04 CDS |
| 642 | DNA | L7-1G04 CDS |
| 643 | DNA | L7-1H04 CDS |
| 644 | DNA | L7-1A05 CDS |
| 645 | DNA | L7-1C05 CDS |
| 646 | DNA | L7-1E05 CDS |
| 647 | DNA | L7-1F05 CDS |
| 648 | DNA | L7-1A06 CDS |
| 649 | DNA | L7-1B06 CDS |
| 650 | DNA | L7-1D06 CDS |
| 651 | DNA | L7-1E06 CDS |
| 652 | DNA | L7-1F06 CDS |
| 653 | DNA | L7-1G06 CDS |
| 654 | DNA | L7-1H06 CDS |
| 655 | DNA | L7-1A07 CDS |
| 656 | DNA | L7-1B07 CDS |
| 657 | DNA | L7-1C07 CDS |
| 658 | DNA | L7-1D07 CDS |
| 659 | DNA | L7-1E07 CDS |
| 660 | DNA | L7-1F07 CDS |
| 661 | DNA | L7-1G07 CDS |
| 662 | DNA | L7-1A08 CDS |
| 663 | DNA | L7-1C08 CDS |
| 664 | DNA | L7-1D08 CDS |
| 665 | DNA | L7-1E08 CDS |
| 666 | DNA | L7-1F08 CDS |
| 667 | DNA | L7-1G08 CDS |
| 668 | DNA | L7-1A09 CDS |
| 669 | DNA | L7-1B09 CDS |
| 670 | DNA | L7-1C09 CDS |
| 671 | DNA | L7-1D09 CDS |
| 672 | DNA | L7-1E09 CDS |
| 673 | DNA | L7-1G09 CDS |
| 674 | DNA | L7-1A10 CDS |
| 675 | DNA | L7-1B10 CDS |
| 676 | DNA | L7-1C10 CDS |
| 677 | DNA | L7-1D10 CDS |
| 678 | DNA | L7-1F10 CDS |
| 679 | DNA | L7-1A11 CDS |
| 680 | DNA | L7-1B11 CDS |
| 681 | DNA | L7-1C11 CDS |
| 682 | DNA | L7-1E11 CDS |
| 683 | DNA | L7-1A12 CDS |
| 684 | DNA | L7-1C12 CDS |
| 685 | DNA | L7-1F12 CDS |
| 686 | DNA | L7-1G12 CDS |
| 687 | DNA | L7-2A01 CDS |
| 688 | DNA | L7-2B01 CDS |
| 689 | DNA | L7-2D01 CDS |
| 690 | DNA | L7-2E01 CDS |
| 691 | DNA | L7-2F01 CDS |
| 692 | DNA | L7-2G01 CDS |
| 693 | DNA | L7-2H01 CDS |
| 694 | DNA | L7-2B02 CDS |
| 695 | DNA | L7-2D02 CDS |
| 696 | DNA | L7-2E02 CDS |
| 697 | DNA | L7-2F02 CDS |
| 698 | DNA | L7-2G02 CDS |
| 699 | DNA | L7-2H02 CDS |
| 700 | DNA | L7-2D03 CDS |
| 701 | DNA | L7-2E03 CDS |
| 702 | DNA | L7-2F03 CDS |
| 703 | DNA | L7-2G03 CDS |
| 704 | DNA | L7-2H03 CDS |
| 705 | DNA | L7-2D04 CDS |
| 706 | DNA | L7-2E04 CDS |
| 707 | DNA | L7-2F04 CDS |
| 708 | DNA | L7-2H04 CDS |
| 709 | DNA | L7-2B05 CDS |
| 710 | DNA | L7-2D05 CDS |
| 711 | DNA | L7-2E05 CDS |
| 712 | DNA | L7-2F05 CDS |
| 713 | DNA | L7-2H05 CDS |
| 714 | DNA | L7-2A06 CDS |
| 715 | DNA | L7-2C06 CDS |
| 716 | DNA | L7-2D06 CDS |
| 717 | DNA | L7-2F06 CDS |
| 718 | DNA | L7-2G06 CDS |
| 719 | DNA | L7-2A07 CDS |
| 720 | DNA | L7-2B07 CDS |
| 721 | DNA | L7-2C07 CDS |
| 722 | DNA | L7-2D07 CDS |
| 723 | DNA | L7-2E07 CDS |
| 724 | DNA | L7-2G07 CDS |
| 725 | DNA | L7-2B08 CDS |
| 726 | DNA | L7-2D08 CDS |
| 727 | DNA | L7-2F08 CDS |
| 728 | DNA | L7-2G08 CDS |
| 729 | DNA | L7-2B09 CDS |
| 730 | DNA | L7-2C09 CDS |
| 731 | DNA | L7-2E09 CDS |
| 732 | DNA | L7-2B10 CDS |
| 733 | DNA | L7-2E10 CDS |
| 734 | DNA | L7-2G10 CDS |
| 735 | DNA | L7-2C11 CDS |
| 736 | DNA | L7-2D11 CDS |
| 737 | DNA | L7-2F11 CDS |
| 738 | DNA | L7-2G11 CDS |
| 739 | DNA | L7-2B12 CDS |
| 740 | DNA | L7-2C12 CDS |
| 741 | DNA | L7-2D12 CDS |
| 742 | DNA | L7-2F12 CDS |
| 743 | DNA | L7-2G12 CDS |
| 744 | DNA | L7-3A01 CDS |
| 745 | DNA | L7-3C01 CDS |
| 746 | DNA | L7-3G01 CDS |
| 747 | DNA | L7-3H01 CDS |
| 748 | DNA | L7-3A02 CDS |
| 749 | DNA | L7-3B02 CDS |
| 750 | DNA | L7-3D02 CDS |
| 751 | DNA | L7-3G02 CDS |
| 752 | DNA | L7-3H02 CDS |
| 753 | DNA | L7-3B03 CDS |
| 754 | DNA | L7-3C03 CDS |
| 755 | DNA | L7-3E03 CDS |
| 756 | DNA | L7-3G03 CDS |
| 757 | DNA | L7-3H03 CDS |
| 758 | DNA | L7-3B04 CDS |
| 759 | DNA | L7-3E04 CDS |
| 760 | DNA | L7-3G04 CDS |
| 761 | DNA | L7-3A05 CDS |
| 762 | DNA | L7-3B05 CDS |
| 763 | DNA | L7-3H05 CDS |
| 764 | DNA | L7-3B06 CDS |
| 765 | DNA | L7-3D06 CDS |
| 766 | DNA | L7-3E06 CDS |
| 767 | DNA | L7-3A07 CDS |
| 768 | DNA | L7-3C07 CDS |
| 769 | DNA | L7-3F07 CDS |
| 770 | DNA | L7-3A08 CDS |
| 771 | DNA | L7-3B08 CDS |
| 772 | DNA | L7-3C08 CDS |
| 773 | DNA | L7-3F08 CDS |
| 774 | DNA | L7-3G08 CDS |
| 775 | DNA | L7-3B09 CDS |
| 776 | DNA | L7-3F09 CDS |
| 777 | DNA | L7-3A10 CDS |
| 778 | DNA | L7-3B10 CDS |
| 779 | DNA | L7-3C10 CDS |
| 780 | DNA | L7-3G10 CDS |
| 781 | DNA | L7-3A11 CDS |
| 782 | DNA | L7-3C11 CDS |
| 783 | DNA | L7-3E11 CDS |
| 784 | DNA | L7-3G11 CDS |
| 785 | DNA | L7-3A12 CDS |
| 786 | DNA | L7-3B12 CDS |
| 787 | DNA | L7-3C12 CDS |
| 788 | DNA | L7-3E12 CDS |
| 789 | DNA | L7-3F12 CDS |
| 790 | DNA | L7-3G12 CDS |
| 791 | DNA | L7-4A01 CDS |
| 792 | DNA | L7-4A03 CDS |
| 793 | DNA | L7-4A04 CDS |
| 794 | DNA | L7-4A06 CDS |
| 795 | DNA | L7-4A08 CDS |
| 796 | DNA | L7-4A09 CDS |
| 797 | DNA | L7-4A12 CDS |
| 798 | DNA | L7-4B03 CDS |
| 799 | DNA | L7-4B04 CDS |
| 800 | DNA | L7-4B06 CDS |
| 801 | DNA | L7-4B07 CDS |
| 802 | DNA | L7-4C01 CDS |
| 803 | DNA | L7-4C03 CDS |
| 804 | DNA | L7-4C04 CDS |
| 805 | DNA | L7-4C06 CDS |
| 806 | DNA | L7-4C09 CDS |
| 807 | DNA | L7-4C12 CDS |
| 808 | DNA | L7-4D04 CDS |
| 809 | DNA | L7-4D07 CDS |
| 810 | DNA | L7-4D08 CDS |
| 811 | DNA | L7-4D10 CDS |
| 812 | DNA | L7-4D11 CDS |
| 813 | DNA | L7-4E01 CDS |
| 814 | DNA | L7-4E02 CDS |
| 815 | DNA | L7-4E04 CDS |
| 816 | DNA | L7-4E05 CDS |
| 817 | DNA | L7-4E07 CDS |
| 818 | DNA | L7-4E08 CDS |
| 819 | DNA | L6-3A09 CDS |
| 820 | DNA | L7-4C06 (E03) CDS |
| 821 | DNA | L10-84 (B12) CDS |
| 822 | DNA | L13-2-46 (D10) CDS |
| 823 | DNA | L12-1-10 CDS |
| 824 | DNA | L13-2-23 CDS |
| 825 | DNA | L7-1C3-A5 |
| 826 | DNA | L7-1F8-A11 |
| 827 | DNA | L7-1G6-B2 |
| 828 | DNA | L7-3E3-D1 |
| 829 | DNA | L1-18 CDS |
| 830 | DNA | L1-21 CDS |
| 831 | DNA | L1-25 CDS |
| 832 | DNA | L1-33 CDS |
| 833 | DNA | L1-34 CDS |
| 834 | DNA | L1-36 CDS |
| 835 | DNA | L1-39 CDS |
| 836 | DNA | L1-41 CDS |
| 841 | DNA | Plasmid PHP37586A |
| 842 | DNA | Plasmid PHP37587A |
| 843 | DNA | Plasmid PHP37588A |
| 844 | DNA | Plasmid PHP37589A |
| 845 | DNA | Plasmid PHP39389A |
| 846 | DNA | Plasmid PHP39390A |
| 847 | DNA | Construct containing |
| | | artificial microRNA |
| 848 | DNA | Tet operator sequence |
| 863 | AA | L13-23 |
| 864 | AA | L15-20 |
| 865 | AA | L15-20-M4 |
| 866 | AA | L15-20-M9 |
| 867 | AA | L15-20-M34 |
| 868 | AA | CsL4.2-20 having the |
| | | L17G mutation |
| 869 | AA | CsL4.2-15 |
| 870 | AA | CsL4.2-20 |
| 884 | AA | L13-23 having the L17G |
| | | mutation |
| 885 | AA | L15-20 having the L17G |
| | | mutation |
| 886 | AA | L15-20-M4 having the |
| | | L17G mutation |
| 887 | AA | L15-20-M9 having the |
| | | L17G mutation |
| 888 | AA | L15-20-M34 having the |
| | | L17G mutation |
| 889 | AA | CsL4.2-15 having the |
| | | L17G mutation |
| 1193 | DNA | L10-11(A04) |
| 1194 | DNA | L10-13(A05) |
| 1195 | DNA | L10-15(A06) |
| 1196 | DNA | L10-30(A09) |
| 1197 | DNA | L10-35(A11) |
| 1198 | DNA | L10-46(B02) |
| 1199 | DNA | L10-47(B03) |
| 1200 | DNA | L10-54(B06) |
| 1201 | DNA | L10-55(B07) |
| 1202 | DNA | L10-59(B08) |
| 1203 | DNA | L10-72(B10) |
| 1204 | DNA | L10-84(B12) |
| 1205 | DNA | L10-90(C02) |
| 1206 | DNA | L11-17(C06) |
| 1207 | DNA | L11-53(C09) |
| 1208 | DNA | L12-1-03 |
| 1209 | DNA | L12-1-06 |
| 1210 | DNA | L12-1-09 |
| 1211 | DNA | L12-1-10 |
| 1212 | DNA | L12-1-11 |
| 1213 | DNA | L12-1-12 |
| 1214 | DNA | L12-1-16 |
| 1215 | DNA | L12-1-17 |
| 1216 | DNA | L12-1-19 |
| 1217 | DNA | L12-1-20 |
| 1218 | DNA | L12-1-21 |
| 1219 | DNA | L12-1-22 |
| 1220 | DNA | L12-2-13 |
| 1221 | DNA | L12-2-14 |
| 1222 | DNA | L12-2-15 |
| 1223 | DNA | L12-2-20 |
| 1224 | DNA | L12-2-22 |
| 1225 | DNA | L12-2-23 |
| 1226 | DNA | L12-2-27 |
| 1227 | DNA | L12-2-33 |
| 1228 | DNA | L12-2-39 |
| 1229 | DNA | L12-2-48 |
| 1230 | DNA | L12-2-49 |
| 1231 | DNA | L12-2-50 |
| 1232 | DNA | L13-1-01 |
| 1233 | DNA | L13-1-02 |
| 1234 | DNA | L13-1-03 |
| 1235 | DNA | L13-1-04 |
| 1236 | DNA | L13-1-05 |
| 1237 | DNA | L13-1-06 |
| 1238 | DNA | L13-1-07 |
| 1239 | DNA | L13-1-08 |
| 1240 | DNA | L13-1-09 |
| 1241 | DNA | L13-1-10 |
| 1242 | DNA | L13-1-11 |
| 1243 | DNA | L13-1-12 |
| 1244 | DNA | L13-1-13 |
| 1245 | DNA | L13-1-14 |
| 1246 | DNA | L13-1-15 |
| 1247 | DNA | L13-1-16 |
| 1248 | DNA | L13-1-17 |
| 1249 | DNA | L13-1-18 |
| 1250 | DNA | L13-1-19 |
| 1251 | DNA | L13-1-20 |
| 1252 | DNA | L13-1-21 |
| 1253 | DNA | L13-1-22 |
| 1254 | DNA | L13-1-23 |
| 1255 | DNA | L13-1-24 |
| 1256 | DNA | L13-1-25 |
| 1257 | DNA | L13-1-26 |
| 1258 | DNA | L13-1-27 |
| 1259 | DNA | L13-1-28 |
| 1260 | DNA | L13-1-29 |
| 1261 | DNA | L13-1-30 |
| 1262 | DNA | L13-1-31 |
| 1263 | DNA | L13-1-32 |
| 1264 | DNA | L13-1-33 |
| 1265 | DNA | L13-1-34 |
| 1266 | DNA | L13-1-35 |
| 1267 | DNA | L13-1-36 |
| 1268 | DNA | L13-1-37 |
| 1269 | DNA | L13-1-38 |
| 1270 | DNA | L13-1-39 |
| 1271 | DNA | L13-1-40 |
| 1272 | DNA | L13-1-41 |
| 1273 | DNA | L13-1-42 |
| 1274 | DNA | L13-1-43 |
| 1275 | DNA | L13-1-44 |
| 1276 | DNA | L13-1-45 |
| 1277 | DNA | L13-1-47 |
| 1278 | DNA | L13-1-48 |
| 1279 | DNA | L13-2-13 |
| 1280 | DNA | L13-2-14 |
| 1281 | DNA | L13-2-15 |
| 1282 | DNA | L13-2-16 |
| 1283 | DNA | L13-2-17 |
| 1284 | DNA | L13-2-18 |
| 1285 | DNA | L13-2-19 |
| 1286 | DNA | L13-2-20 |
| 1287 | DNA | L13-2-21 |
| 1288 | DNA | L13-2-22 |
| 1289 | DNA | L13-2-23 |
| 1290 | DNA | L13-2-24 |
| 1291 | DNA | L13-2-27 |
| 1292 | DNA | L13-2-28 |
| 1293 | DNA | L13-2-29 |
| 1294 | DNA | L13-2-30 |
| 1295 | DNA | L13-2-31 |
| 1296 | DNA | L13-2-32 |
| 1297 | DNA | L13-2-33 |
| 1298 | DNA | L13-2-34 |
| 1299 | DNA | L13-2-35 |
| 1300 | DNA | L13-2-36 |
| 1301 | DNA | L13-2-38 |
| 1302 | DNA | L13-2-39 |
| 1303 | DNA | L13-2-40 |
| 1304 | DNA | L13-2-41 |
| 1305 | DNA | L13-2-42 |
| 1306 | DNA | L13-2-43 |
| 1307 | DNA | L13-2-44 |
| 1308 | DNA | L13-2-45 |
| 1309 | DNA | L13-2-46 |
| 1310 | DNA | L13-2-47 |
| 1311 | DNA | L13-2-48 |
| 1312 | DNA | L13-2-51 |
| 1313 | DNA | L13-2-52 |
| 1314 | DNA | L13-2-53 |
| 1315 | DNA | L13-2-54 |
| 1316 | DNA | L13-2-55 |
| 1317 | DNA | L13-2-56 |
| 1318 | DNA | L13-2-57 |
| 1319 | DNA | L13-2-58 |
| 1320 | DNA | L13-2-59 |
| 1321 | DNA | L13-2-60 |
| 1322 | DNA | L13-2-61 |
| 1323 | DNA | L13-2-62 |
| 1324 | DNA | L13-2-63 |
| 1325 | DNA | L13-2-64 |
| 1326 | DNA | L13-2-65 |
| 1327 | DNA | L13-2-66 |
| 1328 | DNA | L13-2-67 |
| 1329 | DNA | L13-2-68 |
| 1330 | DNA | L13-2-69 |
| 1331 | DNA | L13-2-70 |
| 1332 | DNA | L13-2-71 |
| 1333 | DNA | L13-2-72 |
| 1334 | DNA | L13-2-73 |
| 1335 | DNA | L13-2-74 |
| 1336 | DNA | L13-2-75 |
| 1337 | DNA | L15-01 |
| 1338 | DNA | L15-02 |
| 1339 | DNA | L15-03 |
| 1340 | DNA | L15-04 |
| 1341 | DNA | L15-05 |
| 1342 | DNA | L15-06 |
| 1343 | DNA | L15-07 |
| 1344 | DNA | L15-08 |
| 1345 | DNA | L15-10 |
| 1346 | DNA | L15-11 |
| 1347 | DNA | L15-12 |
| 1348 | DNA | L15-13 |
| 1349 | DNA | L15-14 |
| 1350 | DNA | L15-15 |
| 1351 | DNA | L15-16 |
| 1352 | DNA | L15-17 |
| 1353 | DNA | L15-18 |
| 1354 | DNA | L15-19 |
| 1355 | DNA | L15-20 |
| 1356 | DNA | L15-21 |
| 1357 | DNA | L15-22 |
| 1358 | DNA | L15-23 |
| 1359 | DNA | L15-25 |
| 1360 | DNA | L15-26 |
| 1361 | DNA | L15-27 |
| 1362 | DNA | L15-28 |
| 1363 | DNA | L15-29 |
| 1364 | DNA | L15-30 |
| 1365 | DNA | L15-31 |
| 1366 | DNA | L15-32 |
| 1367 | DNA | L15-33 |
| 1368 | DNA | L15-34 |
| 1369 | DNA | L15-35 |
| 1370 | DNA | L15-36 |
| 1371 | DNA | L15-37 |
| 1372 | DNA | L15-38 |
| 1373 | DNA | L15-39 |
| 1374 | DNA | L15-40 |
| 1375 | DNA | L15-41 |
| 1376 | DNA | L15-42 |
| 1377 | DNA | L15-43 |
| 1378 | DNA | L15-44 |
| 1379 | DNA | L15-45 |
| 1380 | DNA | L15-46 |
| 1381 | AA | L10-11(A04) |
| 1382 | AA | L10-13(A05) |
| 1383 | AA | L10-15(A06) |
| 1384 | AA | L10-30(A09) |
| 1385 | AA | L10-35(A11) |
| 1386 | AA | L10-46(B02) |
| 1387 | AA | L10-47(B03) |
| 1388 | AA | L10-54(B06) |
| 1389 | AA | L10-55(B07) |
| 1390 | AA | L10-59(B08) |
| 1391 | AA | L10-72(B10) |
| 1392 | AA | L10-84(B12) |
| 1393 | AA | L10-90(C02) |
| 1394 | AA | L11-17(C06) |
| 1395 | AA | L11-53(C09) |
| 1396 | AA | L12-1-03 |
| 1397 | AA | L12-1-06 |
| 1398 | AA | L12-1-09 |
| 1399 | AA | L12-1-10 |
| 1400 | AA | L12-1-11 |
| 1401 | AA | L12-1-12 |
| 1402 | AA | L12-1-16 |
| 1403 | AA | L12-1-17 |
| 1404 | AA | L12-1-19 |
| 1405 | AA | L12-1-20 |
| 1406 | AA | L12-1-21 |
| 1407 | AA | L12-1-22 |
| 1408 | AA | L12-2-13 |
| 1409 | AA | L12-2-14 |
| 1410 | AA | L12-2-15 |
| 1411 | AA | L12-2-20 |
| 1412 | AA | L12-2-22 |
| 1413 | AA | L12-2-23 |
| 1414 | AA | L12-2-27 |
| 1415 | AA | L12-2-33 |
| 1416 | AA | L12-2-39 |
| 1417 | AA | L12-2-48 |
| 1418 | AA | L12-2-49 |
| 1419 | AA | L12-2-50 |
| 1420 | AA | L13-1-01 |
| 1421 | AA | L13-1-02 |
| 1422 | AA | L13-1-03 |
| 1423 | AA | L13-1-04 |
| 1424 | AA | L13-1-05 |
| 1425 | AA | L13-1-06 |
| 1426 | AA | L13-1-07 |
| 1427 | AA | L13-1-08 |
| 1428 | AA | L13-1-09 |
| 1429 | AA | L13-1-10 |
| 1430 | AA | L13-1-11 |
| 1431 | AA | L13-1-12 |
| 1432 | AA | L13-1-13 |
| 1433 | AA | L13-1-14 |
| 1434 | AA | L13-1-15 |
| 1435 | AA | L13-1-16 |
| 1436 | AA | L13-1-17 |
| 1437 | AA | L13-1-18 |
| 1438 | AA | L13-1-19 |
| 1439 | AA | L13-1-20 |
| 1440 | AA | L13-1-21 |
| 1441 | AA | L13-1-22 |
| 1442 | AA | L13-1-23 |
| 1443 | AA | L13-1-24 |
| 1444 | AA | L13-1-25 |
| 1445 | AA | L13-1-26 |
| 1446 | AA | L13-1-27 |
| 1447 | AA | L13-1-28 |
| 1448 | AA | L13-1-29 |
| 1449 | AA | L13-1-30 |
| 1450 | AA | L13-1-31 |
| 1451 | AA | L13-1-32 |
| 1452 | AA | L13-1-33 |
| 1453 | AA | L13-1-34 |
| 1454 | AA | L13-1-35 |
| 1455 | AA | L13-1-36 |
| 1456 | AA | L13-1-37 |
| 1457 | AA | L13-1-38 |
| 1458 | AA | L13-1-39 |
| 1459 | AA | L13-1-40 |
| 1460 | AA | L13-1-41 |
| 1461 | AA | L13-1-42 |
| 1462 | AA | L13-1-43 |
| 1463 | AA | L13-1-44 |
| 1464 | AA | L13-1-45 |
| 1465 | AA | L13-1-47 |
| 1466 | AA | L13-1-48 |
| 1467 | AA | L13-2-13 |
| 1468 | AA | L13-2-14 |
| 1469 | AA | L13-2-15 |
| 1470 | AA | L13-2-16 |
| 1471 | AA | L13-2-17 |
| 1472 | AA | L13-2-18 |
| 1473 | AA | L13-2-19 |
| 1474 | AA | L13-2-20 |
| 1475 | AA | L13-2-21 |
| 1476 | AA | L13-2-22 |
| 1477 | AA | L13-2-23 |
| 1478 | AA | L13-2-24 |
| 1479 | AA | L13-2-27 |
| 1480 | AA | L13-2-28 |
| 1481 | AA | L13-2-29 |
| 1482 | AA | L13-2-30 |
| 1483 | AA | L13-2-31 |
| 1484 | AA | L13-2-32 |
| 1485 | AA | L13-2-33 |
| 1486 | AA | L13-2-34 |
| 1487 | AA | L13-2-35 |
| 1488 | AA | L13-2-36 |
| 1489 | AA | L13-2-38 |
| 1490 | AA | L13-2-39 |
| 1491 | AA | L13-2-40 |
| 1492 | AA | L13-2-41 |
| 1493 | AA | L13-2-42 |
| 1494 | AA | L13-2-43 |
| 1495 | AA | L13-2-44 |
| 1496 | AA | L13-2-45 |
| 1497 | AA | L13-2-46 |
| 1498 | AA | L13-2-47 |
| 1499 | AA | L13-2-48 |
| 1500 | AA | L13-2-51 |
| 1501 | AA | L13-2-52 |
| 1502 | AA | L13-2-53 |
| 1503 | AA | L13-2-54 |
| 1504 | AA | L13-2-55 |
| 1505 | AA | L13-2-56 |
| 1506 | AA | L13-2-57 |
| 1507 | AA | L13-2-58 |
| 1508 | AA | L13-2-59 |
| 1509 | AA | L13-2-60 |
| 1510 | AA | L13-2-61 |
| 1511 | AA | L13-2-62 |
| 1512 | AA | L13-2-63 |
| 1513 | AA | L13-2-64 |
| 1514 | AA | L13-2-65 |
| 1515 | AA | L13-2-66 |
| 1516 | AA | L13-2-67 |
| 1517 | AA | L13-2-68 |
| 1518 | AA | L13-2-69 |
| 1519 | AA | L13-2-70 |
| 1520 | AA | L13-2-71 |
| 1521 | AA | L13-2-72 |
| 1522 | AA | L13-2-73 |
| 1523 | AA | L13-2-74 |
| 1524 | AA | L13-2-75 |
| 1525 | AA | L15-01 |
| 1526 | AA | L15-02 |
| 1527 | AA | L15-03 |
| 1528 | AA | L15-04 |
| 1529 | AA | L15-05 |
| 1530 | AA | L15-06 |
| 1531 | AA | L15-07 |
| 1532 | AA | L15-08 |
| 1533 | AA | L15-10 |
| 1534 | AA | L15-11 |
| 1535 | AA | L15-12 |
| 1536 | AA | L15-13 |
| 1537 | AA | L15-14 |
| 1538 | AA | L15-15 |
| 1539 | AA | L15-16 |
| 1540 | AA | L15-17 |
| 1541 | AA | L15-18 |
| 1542 | AA | L15-19 |
| 1543 | AA | L15-20 |
| 1544 | AA | L15-21 |
| 1545 | AA | L15-22 |
| 1546 | AA | L15-23 |
| 1547 | AA | L15-25 |
| 1548 | AA | L15-26 |
| 1549 | AA | L15-27 |
| 1550 | AA | L15-28 |
| 1551 | AA | L15-29 |
| 1552 | AA | L15-30 |
| 1553 | AA | L15-31 |
| 1554 | AA | L15-32 |
| 1555 | AA | L15-33 |
| 1556 | AA | L15-34 |
| 1557 | AA | L15-35 |
| 1558 | AA | L15-36 |
| 1559 | AA | L15-37 |
| 1560 | AA | L15-38 |
| 1561 | AA | L15-39 |
| 1562 | AA | L15-40 |
| 1563 | AA | L15-41 |
| 1564 | AA | L15-42 |
| 1565 | AA | L15-43 |
| 1566 | AA | L15-44 |
| 1567 | AA | L15-45 |
| 1568 | AA | L15-46 |
| 1949 | DNA | L8-1A03 |
| 1950 | DNA | L8-1A04 |
| 1951 | DNA | L8-1A05 |
| 1952 | DNA | L8-1A06 |
| 1953 | DNA | L8-1B12 |
| 1954 | DNA | L8-1C02 |
| 1955 | DNA | L8-1C09 |
| 1956 | DNA | L8-1D03 |
| 1957 | DNA | L8-1D11 |
| 1958 | DNA | L8-1E02 |
| 1959 | DNA | L8-1E04 |
| 1960 | DNA | L8-2A08 |
| 1961 | DNA | L8-2B05 |
| 1962 | DNA | L8-2F04 |
| 1963 | DNA | L8-2F10 |
| 1964 | DNA | L8-2F12 |
| 1965 | DNA | L8-2H01 |
| 1966 | DNA | L8-3A04 |
| 1967 | DNA | L8-3A05 |
| 1968 | DNA | L8-3A06 |
| 1969 | DNA | L8-3A07 |
| 1970 | DNA | L8-3A10 |
| 1971 | DNA | L8-3A12 |
| 1972 | DNA | L8-3B02 |
| 1973 | DNA | L8-3B03 |
| 1974 | DNA | L8-3B05 |
| 1975 | DNA | L8-3B08 |
| 1976 | DNA | L8-3B09 |
| 1977 | DNA | L8-3D03 |
| 1978 | DNA | L8-3D04 |
| 1979 | DNA | L8-3D12 |
| 1980 | DNA | L8-3E05 |
| 1981 | DNA | L8-3E09 |
| 1982 | DNA | L8-3F01 |
| 1983 | DNA | L8-3F02 |
| 1984 | DNA | L8-3F06 |
| 1985 | DNA | L8-3F08 |
| 1986 | DNA | L8-3F09 |
| 1987 | DNA | CsL3-1A07 |
| 1988 | DNA | CsL3-1B04 |
| 1989 | DNA | CsL3-1B05 |
| 1990 | DNA | CsL3-1B11 |
| 1991 | DNA | CsL3-1C01 |
| 1992 | DNA | CsL3-1C12 |
| 1993 | DNA | CsL3-2A01 |
| 1994 | DNA | CsL3-2B06 |
| 1995 | DNA | CsL3-2B09 |
| 1996 | DNA | CsL3-2B12 |
| 1997 | DNA | CsL3-2D02 |
| 1998 | DNA | CsL3-2D10 |
| 1999 | DNA | CsL3-2D11 |
| 2000 | DNA | CsL3-2D12 |
| 2001 | DNA | CsL3-2E07 |
| 2002 | DNA | CsL3-2E08 |
| 2003 | DNA | CsL3-2E09 |
| 2004 | DNA | CsL3-2E10 |
| 2005 | DNA | CsL3-2E11 |
| 2006 | DNA | CsL3-2E12 |
| 2007 | DNA | CsL3-MTZ2 |
| 2008 | DNA | CsL3-MTZ3 |
| 2009 | DNA | CsL3-MTZ4 |
| 2010 | DNA | CsL3-MTZ5 |
| 2011 | DNA | CsL4.2-01 |
| 2012 | DNA | CsL4.2-04 |
| 2013 | DNA | CsL4.2-07 |
| 2014 | DNA | CsL4.2-08 |
| 2015 | DNA | CsL4.2-11 |
| 2016 | DNA | CsL4.2-12 |
| 2017 | DNA | CsL4.2-15 |
| 2018 | DNA | CsL4.2-16 |
| 2019 | DNA | CsL4.2-17 |
| 2020 | DNA | CsL4.2-18 |
| 2021 | DNA | CsL4.2-20 |
| 2022 | DNA | CsL4.2-21 |
| 2023 | DNA | CsL4.2-22 |
| 2024 | DNA | CsL4.2-23 |
| 2025 | DNA | CsL4.2-24 |
| 2026 | DNA | CsL4.2-26 |
| 2027 | DNA | CsL4.2-27 |
| 2028 | DNA | CsL4.2-28 |
| 2029 | DNA | CsL4.2-30 |
| 2030 | AA | L8-1A03 |
| 2031 | AA | L8-1A04 |
| 2032 | AA | L8-1A05 |
| 2033 | AA | L8-1A06 |
| 2034 | AA | L8-1B12 |
| 2035 | AA | L8-1C02 |
| 2036 | AA | L8-1C09 |
| 2037 | AA | L8-1D03 |
| 2038 | AA | L8-1D11 |
| 2039 | AA | L8-1E02 |
| 2040 | AA | L8-1E04 |
| 2041 | AA | L8-2A08 |
| 2042 | AA | L8-2B05 |
| 2043 | AA | L8-2F04 |
| 2044 | AA | L8-2F10 |
| 2045 | AA | L8-2F12 |
| 2046 | AA | L8-2H01 |
| 2047 | AA | L8-3A04 |
| 2048 | AA | L8-3A05 |
| 2049 | AA | L8-3A06 |
| 2050 | AA | L8-3A07 |
| 2051 | AA | L8-3A10 |
| 2052 | AA | L8-3A12 |
| 2053 | AA | L8-3B02 |
| 2054 | AA | L8-3B03 |
| 2055 | AA | L8-3B05 |
| 2056 | AA | L8-3B08 |
| 2057 | AA | L8-3B09 |
| 2058 | AA | L8-3D03 |
| 2059 | AA | L8-3D04 |
| 2060 | AA | L8-3D12 |
| 2061 | AA | L8-3E05 |
| 2062 | AA | L8-3E09 |
| 2063 | AA | L8-3F01 |
| 2064 | AA | L8-3F02 |
| 2065 | AA | L8-3F06 |
| 2066 | AA | L8-3F08 |
| 2067 | AA | L8-3F09 |
| 2068 | AA | CsL3-1A07 |
| 2069 | AA | CsL3-1B04 |
| 2070 | AA | CsL3-1B05 |
| 2071 | AA | CsL3-1B11 |
| 2072 | AA | CsL3-1C01 |
| 2073 | AA | CsL3-1C12 |
| 2074 | AA | CsL3-2A01 |
| 2075 | AA | CsL3-2B06 |
| 2076 | AA | CsL3-2B09 |
| 2077 | AA | CsL3-2B12 |
| 2078 | AA | CsL3-2D02 |
| 2079 | AA | CsL3-2D10 |
| 2080 | AA | CsL3-2D11 |
| 2081 | AA | CsL3-2D12 |
| 2082 | AA | CsL3-2E07 |
| 2083 | AA | CsL3-2E08 |
| 2084 | AA | CsL3-2E09 |
| 2085 | AA | CsL3-2E10 |
| 2086 | AA | CsL3-2E11 |
| 2087 | AA | CsL3-2E12 |
| 2088 | AA | CsL3-MTZ2 |
| 2089 | AA | CsL3-MTZ3 |
| 2090 | AA | CsL3-MTZ4 |
| 2091 | AA | CsL3-5 |
| 2092 | AA | CsL4.2-01 |
| 2093 | AA | CsL4.2-04 |
| 2094 | AA | CsL4.2-07 |
| 2095 | AA | CsL4.2-08 |
| 2096 | AA | CsL4.2-11 |
| 2097 | AA | CsL4.2-12 |
| 2098 | AA | CsL4.2-15 |
| 2099 | AA | CsL4.2-16 |
| 2100 | AA | CsL4.2-17 |
| 2101 | AA | CsL4.2-18 |
| 2102 | AA | CsL4.2-20 |
| 2103 | AA | CsL4.2-21 |
| 2104 | AA | CsL4.2-22 |
| 2105 | AA | CsL4.2-23 |
| 2106 | AA | CsL4.2-24 |
| 2107 | AA | CsL4.2-26 |
| 2108 | AA | CsL4.2-27 |
| 2109 | AA | CsL4.2-28 |
| 2110 | AA | CsL4.2-30 |
| 2111 | DNA | PHP46916 |
| 2112 | DNA | PHP46864 |
| 2113 | DNA | PHP1194 |
| 2114 | DNA | PHP1195 |
| 2115 | DNA | PHP1196 |
| 2116 | DNA | PHP1197 |
| 2117 | DNA | PHP1198 |
| 2118 | DNA | PHP1199 |

The following examples are provided to illustrate some embodiments of the invention, but should not be construed as defining or otherwise limiting any aspect, embodiment, element or any combinations thereof. Modifications of any aspect, embodiment, element or any combinations thereof are apparent to a person of skill in the art.

### EXPERIMENTAL

### Example 1

### Use of a Sulfonylurea Repressor Controlled siRNA Targeted Against the Sulfonylurea Repressor for Amplification and Increased Spatial Distribution of Induced Signal

One of the main limitations of any chemically inducible system in multicellular organisms is the penetration and even distribution of the inducer throughout all tissues (due to variable movement or metabolism). The result is the possibility of uneven (or lack of) targeted gene induction in the tissues or cell types of interest. To address this potential caveat, it is desired to provide additional genetic factors to effect the spread of de-repression. siRNA's have been used extensively in eukaryotic systems to knockdown targeted gene expression. In particular plants have the added potential that the siRNA response can go systemic (Palauqui et al. (1997) EMBO J. 16: 4738-4745; Voinnet et al. (1997) Nature 389: 553) depending on the type of silencing signal generated (Felipe Fenselau de Felippes et al. (2010) Nucleic Acids Research 1-10).

Thus a well suited approach for enhancing spatial spread of signal in plants using the SuR based switch is to control repressor transcript stability through de-repression of a mobile siRNA generating signal targeted against any or all parts of the transcript harboring the repressor coding region. Auto-inducing regulating repressor expression thru siRNA has been demonstrated in mammalian cell cultures (Greber et al. (2008) Nucleic Acids Research 36: 16). In the above example, it was shown that induction of an siRNA against the repressor greatly extended the time period of the induced state following removal of ligand. However, this study was limited to tissue culture cells and not extended to a whole animal model where the inducer is unlikely to contact all cell types following administration. Furthermore, unlike plants, higher animals are not known to communicate siRNA signals systemically and thus the aspect of enhancing induction spatially may not translate to animal systems.

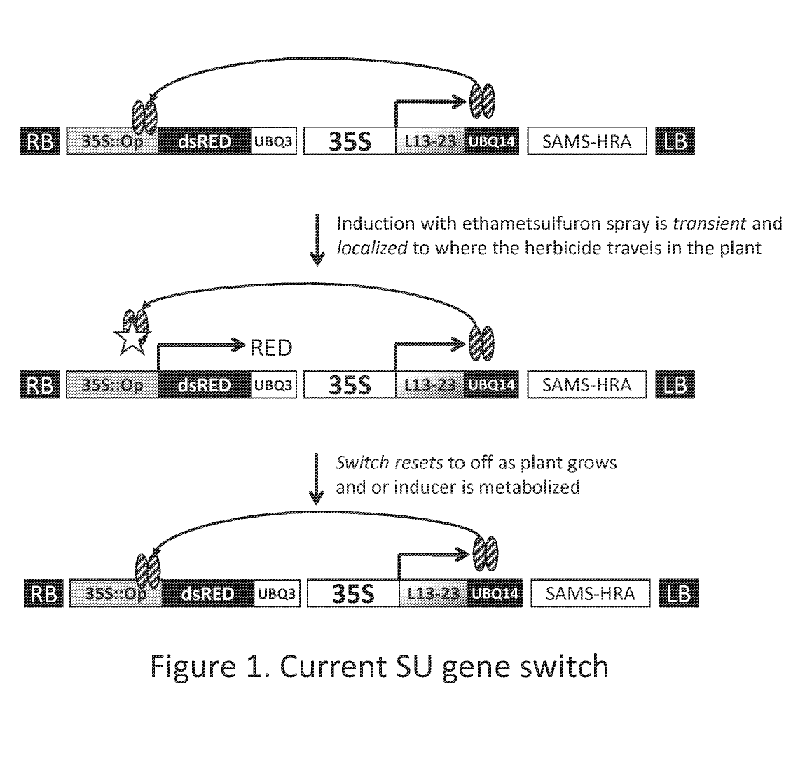

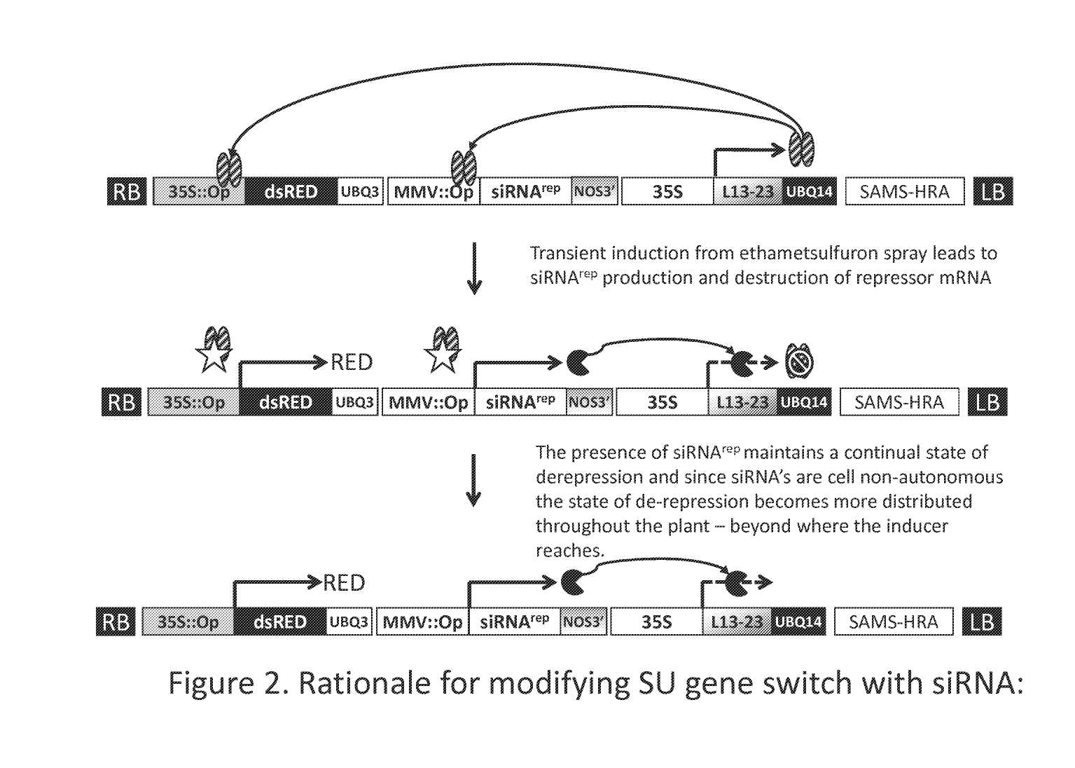

This method can be tested by adding to the SU switch, as exemplified in FIG. 1, an expression cassette having a tetO controlled promoter linked to an siRNA that is targeted to the repressor transcript (siRNA^{rep}; FIG. 2). Because siRNA^{rep} can lead to systemic spread of the silencing signal, de-repression would spread well beyond the bounds of the inducer. The cell non-autonomous feature of this method thus clearly differentiates it over other possible techniques to extend and intensify de-repression.

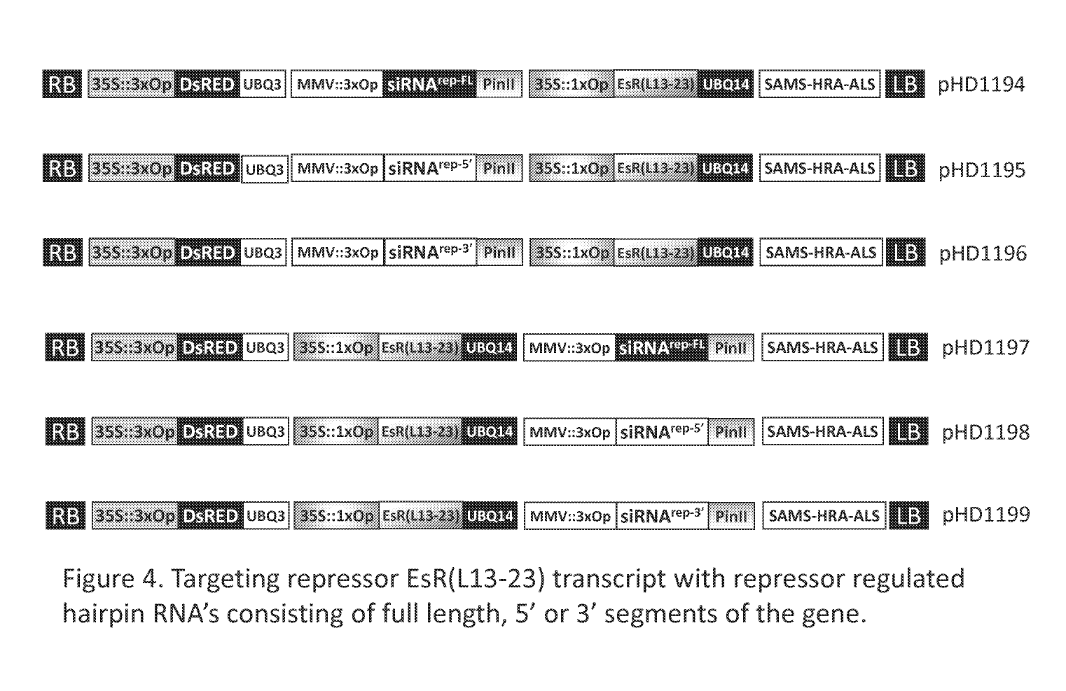

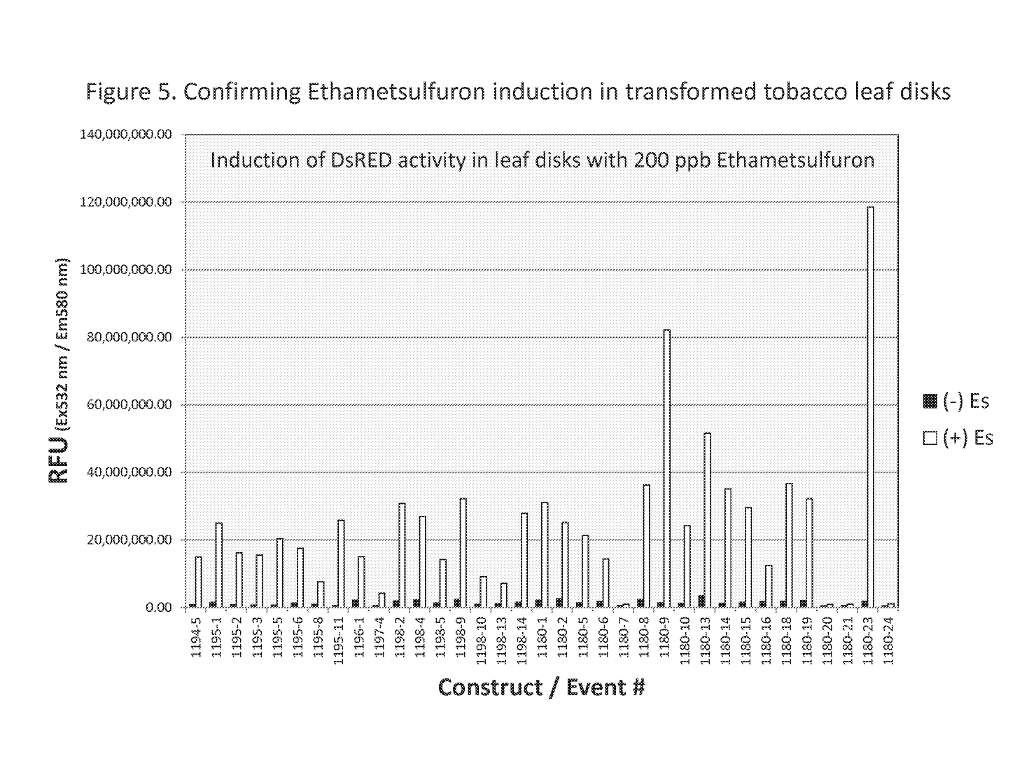

To test this principle, inducible lines of tobacco harboring constructs shown in FIG. 4 were created. A summary of the constructs shown in FIG. 4 is provided below in table 29. All vectors contain a right border (RB) proximal 35S::3xtetO-DsRED-UBQ3 inducible reporter cassette, a 35S::1xtetO-EsR(L13-32)-UBQ14 repressor cassette, and a SAMS-HRA-ALS left border (LB) proximal selectable marker. Inserted either upstream (pHD1194-1196) or downstream (pHD1196-1199) of the repressor cassette are MMV::3xOp-siRNA^{rep}-Pin2 cassettes composed of an inverted repeat of the full length repressor coding region (no ATG-pHD1194 and 1197) or limited to the 5′ (pHD1195 & 1198) or 3′ (pHD1196 & 1199) halves of the SU repressor coding region linked by an intron spacer region. The MMV::tetO promoter was chosen so as not to cause silencing of the 35S::tetO promoter controlling target transgene expression. In this particular example the spacer region is the potato ST-LS1 gene intron IV2 (Construction of an intron-containing marker gene: splicing of the intron in transgenic plants and its use in monitoring early events in Agrobacterium-mediated plant transformation. Vancanneyt et al. (1990) Mol Gen Genet. 220(2):245-50). The vectors were transferred to A. tumefaciens EHA105 and transformed into leaf explants of wild type Nicotiana tabacum via Agrobacterium co-cultivation followed by selection for the presence of the HRA marker gene on 50 ppb imazapyr (inhibitor of acetolactate synthase but non-inducer of the SuR system). Duplicate excised leaf disks from each transformant were screened for controlled dsRED gene expression in the absence and presence of inducer Ethametsulfuron-methyl at 50 ppb (FIG. 5). T1 seeds from each of the inducible events were allowed to germinate and on filter paper contacting 0.5xMS agar with 1 ppm Ethametsulfuron. Nine fully derepressed DsRED positive seedings for each event were then transplanted into soil and their fluorescence phenotype monitored thru the four leaf developmental stage. Inducible tobacco events harboring pHD1180 (isogenic to vectors pHD1194-1199 but without the siRNA cassette) were used as the controls. Results show that while the DsRED expression signal is modest and diminishes in pHD1180 events over time, the DsRED intensity level is high and remains so with time in lines containing the MMV::tetO-siRNA^{rep} cassette (FIG. 6).

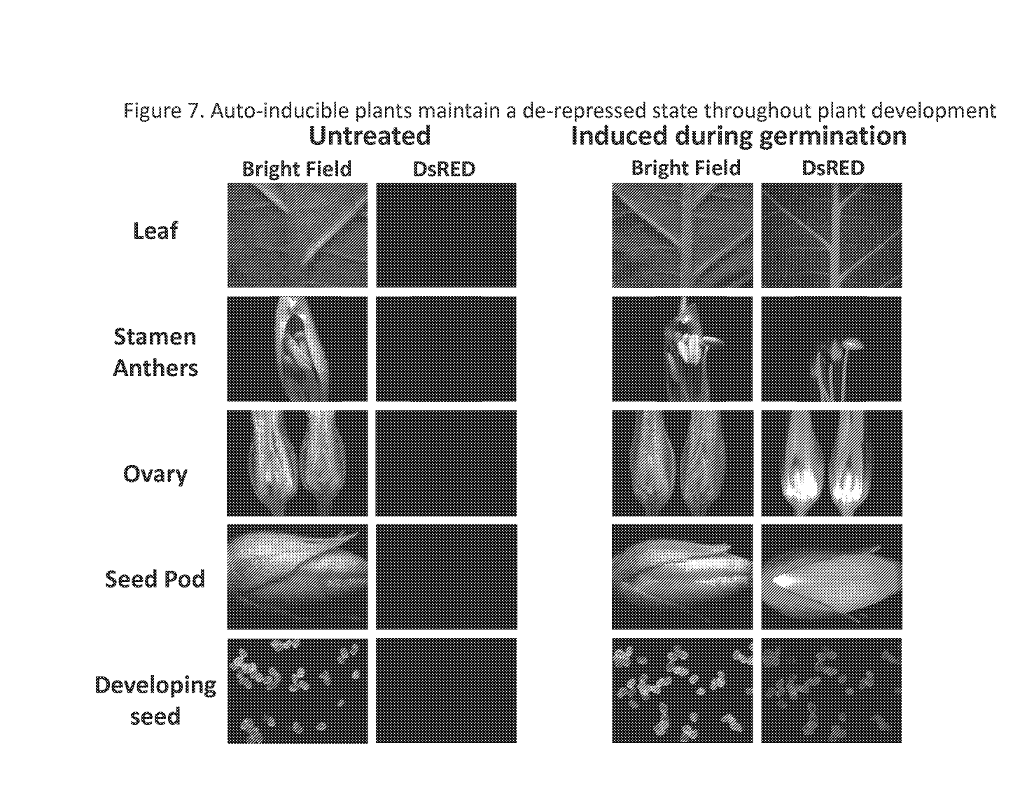

In a second experiment T1 seed of auto-inducible line pHD1198-2 were planted in soil treated with water or a one-time application of 20 ml of Muster (commercial form of Ethametsulfuron-methyl—DuPont) made up to a 1/16^{th}× recommended spray rate concentration in water. The DsRED phenotype was then followed throughout the plants entire life cycle. The results show that DsRED is fully activated throughout the life span of the plant and in all tissues examined except pollen (35S promoter silent in pollen) in the Muster treated plant but silent in control plants treated only with water (FIG. 7). Most notably the DsRED phenotype persists all the way thru seed development.

TABLE 29

| Plasmid | Cassette | Element | Nt position in plasmid |
|---|---|---|---|
| pHD1194 (SEQ | cassette A | 35S::3xOp | 177-623 |
| ID NO: 2113) | | DsRED | 699-1373 |
| | | UBQ3 | 1392-2500 |
| | cassette B | MMV::3xOp | 2600-2929 |
| | | siRNA rep-FL | 2936-4273 |
| | | PinII | 4380-4690 |
| | cassette C | 35S::1xOp | 4697-5218 |
| | | EsR(L13-23) | 5219-5839 |
| | | UBQ14 | 5883-6784 |
| | cassette D | SAMS | 6906-8215 |
| | | HRA | 8216-10186 |
| | | ALS | 10187-10837 |
| pHD1195 (SEQ | cassette A | 35S::3xOp | 177-623 |
| ID NO: 2114) | | DsRED | 699-1373 |
| | | UBQ3 | 1392-2500 |
| | cassette B | MMV::3xOp | 2600-2929 |
| | | siRNA rep-5′ | 2936-3759 |
| | | PinII | 3766-4076 |
| | cassette C | 35S::1xOp | 4083-4604 |
| | | EsR(L13-23) | 4605-5225 |
| | | UBQ14 | 5269-6170 |
| | cassette D | SAMS | 6292-7601 |
| | | HRA | 7602-9572 |
| | | ALS | 9573-10223 |
| pHD1196 (SEQ | cassette A | 35S::3xOp | 177-623 |
| ID NO: 2115) | | DsRED | 699-1373 |
| | | UBQ3 | 1392-2500 |
| | cassette B | MMV::3xOp | 2600-2929 |
| | | siRNA rep-3′ | 2936-3755 |
| | | PinII | 3762-4072 |
| | cassette C | 35S::1xOp | 4079-4600 |
| | | EsR(L13-23) | 4601-5221 |
| | | UBQ14 | 5265-6166 |
| | cassette D | SAMS | 6288-7597 |
| | | HRA | 7598-9568 |
| | | ALS | 9569-10219 |
| phD1197 SEQ | cassette A | 35S::3xOp | 177-623 |
| ID NO: 2116) | | DsRED | 699-1373 |
| | | UBQ3 | 1392-2500 |
| | cassette B | 35S::1xOp | 2590-3111 |
| | | EsR(L13-23) | 3112-3732 |
| | | UBQ14 | 3776-4677 |
| | cassette C | MMV::3xOp | 4708-5037 |
| | | siRNA rep-FL | 5044-6481 |
| | | PinII | 6488-6798 |
| | cassette D | SAMS | 6922-8231 |
| | | HRA | 8232-10202 |
| | | ALS | 10203-10853 |
| phD1198 SEQ | cassette A | 35S::3xOp | 177-623 |
| ID NO: 2117) | | DsRED | 699-1373 |
| | | UBQ3 | 1392-2500 |
| | cassette B | 35S::1xOp | 2590-3111 |
| | | EsR(L13-23) | 3112-3732 |
| | | UBQ14 | 3776-4677 |
| | cassette C | MMV::3xOp | 4708-5037 |
| | | siRNA rep-5′ | 5044-5867 |
| | | PinII | 5874-6184 |
| | cassette D | SAMS | 6308-7617 |
| | | HRA | 7618-9588 |
| | | ALS | 9589-10239 |
| phD1199 SEQ | cassette A | 35S::3xOp | 177-623 |
| ID NO: 2118) | | DsRED | 699-1373 |
| | | UBQ3 | 1392-2500 |
| | cassette B | 35S::1xOp | 2590-3111 |
| | | EsR(L13-23) | 3112-3732 |
| | | UBQ14 | 3776-4677 |
| | cassette C | MMV::3xOp | 4708-5037 |
| | | siRNA rep-3′ | 5044-5863 |
| | | PinII | 5870-6180 |
| | cassette D | SAMS | 6304-7613 |
| | | HRA | 7614-9584 |
| | | ALS | 9585-10235 |

### Example 2

### Use of a Sulfonylurea Repressor Controlled miRNA Targeted Against the Sulfonylurea Repressor for Amplification and Increased Spatial Distribution of Induced Signal

To show that the presence of an amiRNA targeted against the repressor protein increases expression after induction two constructs were made. The first, a control construct, pPHP46916 (10,904 bp) (SEQ ID NO: 2111) contains the following cassettes: cassette A comprising a Glycine max s-adenosylmethionine promoter operably linked to the Glycine max acetolactate synthase gene with HrA mutations operably linked to a Glycine max acetolactate synthase terminator (this cassette serves as a selectable marker during plant transformation; position 81-4062); followed by cassette B comprising the T7 promoter operably linked to hygromycin phosphotransferase operably linked to a T7 terminator (which serves as a selectable marker in E. coli, positions 5448-6586); followed by cassette C comprising a cauliflower mosaic virus 35S promoter with three copies of the TET operator embedded operably linked to DS-RED Express that has the potato LS1 intron; operably linked to the cauliflower mosaic virus 35S terminator (position 6862-8455), followed by cassette D comprising the Glycine max elongation factor 1a2 promoter operably linked to the repressor protein ESR (L10-B7) operably linked to the nos terminator (position 8474-10893). The second, experimental construct, pPHP46864 (11,868 bp) (SEQ ID NO:2112) is exactly the same except embedded within the potato LS1 intron at the Mfe1 site is a 964 bp cassette containing the Glycine max microRNA precursor 159 containing a microRNA that targets the repressor protein. The microRNA precursor and the design procedure are explained in US 2011-0091975, the contents of which are herein incorporated by reference in its entirety.

TABLE 28

| Plasmid | Cassette | Element | Nt position in plasmid |
|---|---|---|---|
| pPHP46916 (SEQ | Cassette A | | 81-4062 |
| ID NO: 2111) | | Glycine max s-adenosylmethionine promoter | 81-1389 |
| | | Glycine max acetolactate synthase gene with | 1456-3411 |
| | | HrA mutations | |
| | | Glycine max acetolactate synthase terminator | 3412-4062 |
| | Cassette B | | 5448-6733 |
| | | T7 promoter operably linked to | 5448-5545 |
| | | hygromycin phosphotransferase | 5546-6586 |
| | | T7 terminator | 6587-6733 |
| | Cassette C | | 6862-8455 |
| | | cauliflower mosaic virus 35S promoter with | 6862-7378 |
| | | three copies of the TET operator | |
| | | DS-RED Express that has the potato LS1 | 7385-8251 |
| | | intron | |
| | | operably linked to the cauliflower mosaic | 8258-8455 |
| | | virus 35S terminator | |
| | cassette D | | 8474-10893 |
| | | Glycine max elongation factor 1a2 promoter | 8474-9974 |
| | | repressor protein ESR (L10-B7) | 9976-10596 |
| | | operably linked to the nos terminator | 10613-10893 |
| pPHP46864 (SEQ | Cassette A | | 74-4055 |
| ID NO: 2112) | | Glycine max s-adenosylmethionine promoter | 74-1382 |
| | | Glycine max acetolactate synthase gene with | 1449-3404 |
| | | HrA mutations | |
| | | Glycine max acetolactate synthase terminator | 3405-4055 |
| | Cassette B | | 5441-6726 |
| | | T7 promoter operably linked to | 5441-5538 |
| | | hygromycin phosphotransferase | 5539-6579 |
| | | T7 terminator | 6580-6726 |
| | Cassette C | | 6855-9412 |
| | | cauliflower mosaic virus 35S promoter with | 6855-7371 |
| | | three copies of the TET operator | |
| | | DS-RED Express that has the potato LS1 | 7378-9208 |
| | | intron and within the potato LS1 intron at the | |
| | | Mfe1 site is a 964 bp cassette containing the | |
| | | Glycine max microRNA precursor 159 | |
| | | containing a microRNA that targets the | |
| | | repressor protein | |
| | | operably linked to the cauliflower mosaic | 9215-9412 |
| | | virus 35S terminator | |
| | cassette D | | 9431-11850 |
| | | Glycine max elongation factor 1a2 promoter | 9431-10931 |
| | | repressor protein ESR (L10-B7) | 10933-11553 |
| | | operably linked to the nos terminator | 11570-11850 |

From both plasmids a fragment of DNA containing all of the described cassettes except for the bacterial selection was made and used to transform soybean as described in Example 3. Plants were selected and leaf discs were obtained at the T0 plant stage. The leaf discs were floated in tissue culture media with 0, 0.05 ppm or 0.5 ppm ethametsulfuron at room temperature for 3-4 days and observed under a fluorescent microscope. A range of phenotypes was observed in different genetically distinct events including events that were leaky (i.e., leaf discs showed DS-RED expression without induction) and leaf discs that were not able to be induced (i.e., leaf discs never showed DS-RED expression). However, among the leaf discs that were able to be induced the leaf discs from the experimental plants showed a smoother, more even pattern of expression.

T0 plants were allowed to mature and seed was collected. This T1 seed was imbibed with 1 ppm chlorsulfuron and planted in a growth chamber and examined under a fluorescent microscope at two weeks which is just as the first trifoliate is appearing. Some of the plants show DS-red positive. For the control plants examined there was no DS-red signal found in root, stem or cotyledon. For experimental plants there was a weak DS-red signal can only be observed in root, stem and an even weaker signal in the cotyledon. This shows that the presence of the amiRNA targeting the repressor increases both the intensity and the domain of the reporter.

Chlorsulfuron works best when part of a formulation. Because of that we used the commercial product Tevlar XP (which is 75% chlorsulfuron). T1 Seeds were planted and watered for about 10 days and then watered with at day 11 and day 14 with a 0.2 gram/liter Tevlar XP. At day 18 the plants were examined under a fluorescent microscope. In plants derived from the experimental plasmid, there was strong induction throughout the seedling except in the cotyledons while the plants derived from the control plasmid showed only a small amount of induction in the root. The plants were allowed to grow for an additional two weeks only being watered (no Tevlar) and experimental plants continued to show a strong pattern of induction throughout the plant as opposed to the control plants that showed little or no expression and only in roots. This shows that the presence of the amiRNA targeting the repressor increases both the intensity and the domain of the reporter.

### Example 3

### Production and Model System Transformation of Somatic Soybean Embryo Cultures with Soybean Expression Vectors and Plant Regeneration

### Culture Conditions:

Soybean embryogenic suspension cultures (cv. Jack) are maintained in 35 mL liquid medium SB196 (infra) on a rotary shaker, 150 rpm, 26° C. with cool white fluorescent lights on 16:8 hr day/night photoperiod at light intensity of 60-85 μE/m2/s. Cultures are subcultured every 7 days to two weeks by inoculating approximately 35 mg of tissue into 35 mL of fresh liquid SB196 (the preferred subculture interval is every 7 days).

Soybean embryogenic suspension cultures are transformed with the soybean expression plasmids by the method of particle gun bombardment (Klein et al., Nature 327:70 (1987)) using a DuPont Biolistic PDS1000/HE instrument (helium retrofit) for all transformations.

### Soybean Embryogenic Suspension Culture Initiation:

Soybean cultures are initiated twice each month with 5-7 days between each initiation. Pods with immature seeds from available soybean plants are picked 45-55 days after planting. Seeds are removed from the pods and placed into a sterilized magenta box. The soybean seeds are sterilized by shaking them for 15 min in a 5% Clorox solution with 1 drop of Ivory soap (i.e., 95 mL of autoclaved distilled water plus 5 mL Clorox and 1 drop of soap, mixed well). Seeds are rinsed using 2 1-liter bottles of sterile distilled water and those less than 4 mm are placed on individual microscope slides. The small end of the seed is cut and the cotyledons pressed out of the seed coat. When cultures are being prepared for production transformation, cotyledons are transferred to plates containing SB1 medium (25-30 cotyledons per plate). Plates are wrapped with fiber tape and are maintained at 26° C. with cool white fluorescent lights on 16:8 h day/night photoperiod at light intensity of 60-80 μE/m2/s for eight weeks, with a media change after 4 weeks. When cultures are being prepared for model system experiments, cotyledons are transferred to plates containing SB 199 medium (25-30 cotyledons per plate) for 2 weeks, and then transferred to SB1 for 2-4 weeks. Light and temperature conditions are the same as described above. After incubation on SB1 medium, secondary embryos are cut and placed into SB196 liquid media for 7 days.

### Preparation of DNA for Bombardment:

Either an intact plasmid or a DNA plasmid fragment containing the genes of interest and the selectable marker gene are used for bombardment. Fragments from soybean expression plasmids are obtained by gel isolation of digested plasmids. In each case, 100 μg of plasmid DNA is used in 0.5 mL of the specific enzyme mix described below. Plasmids are digested with AscI (100 units) in NEBuffer 4 (20 mM Tris-acetate, 10 mM magnesium acetate, 50 mM potassium acetate, 1 mM dithiothreitol, pH 7.9), 100 μg/mL BSA, and 5 mM beta-mercaptoethanol at 37° C. for 1.5 h. The resulting DNA fragments are separated by gel electrophoresis on 1% SeaPlaque GTG agarose (BioWhitaker Molecular Applications) and the DNA fragments containing gene cassettes are cut from the agarose gel. DNA is purified from the agarose using the GELase digesting enzyme following the manufacturer's protocol.

A 50 μL aliquot of sterile distilled water containing 3 mg of gold particles (3 mg gold) is added to 30 μL of a 10 ng/μL DNA solution (either intact plasmid or DNA fragment prepared as described herein), 25 μL 5M CaCl₂ and 20 μL of 0.1 M spermidine. The mixture is shaken 3 min on level 3 of a vortex shaker and spun for 10 sec in a bench microfuge. The supernatant is removed, followed by a wash with 400 μL 100% ethanol and another brief centrifugation. The 400 μL ethanol is removed and the pellet is resuspended in 40 μL of 100% ethanol. Five μL of DNA suspension is dispensed to each flying disk of the Biolistic PDS1000/HE instrument disk. Each 5 μL aliquot contains approximately 0.375 mg gold per bombardment (e.g., per disk).

For model system transformations, the protocol is identical except for a few minor changes (i.e., 1 mg of gold particles is added to 5 μL of a 1 μg/μL DNA solution, 50 μL of a 2.5M CaCl₂ is used and the pellet is ultimately resuspended in 85 μL of 100% ethanol thus providing 0.058 mg of gold particles per bombardment).

Tissue Preparation and Bombardment with DNA:

Approximately 150-200 mg of seven day old embryogenic suspension cultures is placed in an empty, sterile 60×15 mm petri dish and the dish is covered with plastic mesh. The chamber is evacuated to a vacuum of 27-28 inches of mercury, and tissue is bombarded one or two shots per plate with membrane rupture pressure set at 1100 PSI. Tissue is placed approximately 3.5 inches from the retaining/stopping screen. Model system transformation conditions are identical except 100-150 mg of embryogenic tissue is used, rupture pressure is set at 650 PSI and tissue is place approximately 2.5 inches from the retaining screen.

### Selection of Transformed Embryos:

Transformed embryos are selected either using hygromycin (when the hygromycin B phosphotransferase (HPT) gene is used as the selectable marker) or chlorsulfuron (when the acetolactate synthase (ALS) gene is used as the selectable marker).

Following bombardment, the tissue is placed into fresh SB 196 media and cultured as described above. Six to eight days post-bombardment, the SB196 is exchanged with fresh SB196 containing either 30 mg/L hygromycin or 100 ng/mL chlorsulfuron, depending on the selectable marker used. The selection media is refreshed weekly. Four to six weeks post-selection, green, transformed tissue is observed growing from untransformed, necrotic embryogenic clusters.

### Embryo Maturation:

For production transformations, isolated, green tissue is removed and inoculated into multiwell plates to generate new, clonally propagated, transformed embryogenic suspension cultures. Transformed embryogenic clusters are cultured for four-six weeks in multiwell plates at 26° C. in SB 196 under cool white fluorescent (Phillips cool white Econowatt F40/CW/RS/EW) and Agro (Phillips F40 Agro) bulbs (40 watt) on a 16:8 hr photoperiod with light intensity of 90-120 μE/m²s. After this time embryo clusters are removed to a solid agar media, SB 166, for one-two weeks and then subcultured to SB103 medium for 3-4 weeks to mature embryos. After maturation on plates in SB 103, individual embryos are removed from the clusters, dried and screened for a desired phenotype.

For model system transformations, embryos are matured in soybean histodifferentiation and maturation liquid medium (SHaM liquid media; Schmidt et al., Cell Biology and Morphogenesis 24:393 (2005)) using a modified procedure. Briefly, after 4 weeks of selection in SB196 as described above, embryo clusters are removed to 35 mL of SB228 (SHaM liquid media) in a 250 mL Erlenmeyer flask. Tissue is maintained in SHaM liquid media on a rotary shaker at 130 rpm and 26° C. with cool white fluorescent lights on a 16:8 hr day/night photoperiod at a light intensity of 60-85 μE/m2/s for 2 weeks as embryos mature. Embryos grown for 2 weeks in SHaM liquid media are equivalent in size and fatty acid content to embryos cultured on SB166/SB103 for 5-8 weeks.

| 1. | Media Recipes: | | |
|---|---|---|---|
| 2. | SB 196 - FN Lite Liquid Proliferation Medium (per liter) | | |
| | MS FeEDTA - 100x Stock 1 | 10 | mL |
| | MS Sulfate - 100x Stock 2 | 10 | mL |
| | FN Lite Halides - 100x Stock 3 | 10 | mL |
| | FN Lite P, B, Mo - 100x Stock 4 | 10 | mL |
| | B5 vitamins (1 mL/L) | 1.0 | mL |
| | 2,4-D (10 mg/L final concentration) | 1.0 | mL |
| | KNO₃ | 2.83 | gm |
| | (NH₄)₂SO₄ | 0.463 | gm |
| | asparagine | 1.0 | gm |
| | sucrose (1%) | 10 | gm |
| | pH 5.8 | | |

FN Lite Stock Solutions

| | | | | | |
|---|---|---|---|---|---|
| | Stock Number | 1000 | mL | 500 | mL |
| 3. | 1 MS Fe EDTA 100x Stock | | | | |
| | Na₂ EDTA* | 3.724 | g | 1.862 | g |
| | FeSO₄—7H₂O | 2.784 | g | 1.392 | g |

*Add first, dissolve in dark bottle while stirring

| | | | | | |
|---|---|---|---|---|---|
| | 2 MS Sulfate 100x stock | | | | |
| | MgSO₄—7H₂O | 37.0 | g | 18.5 | g |
| | MnSO₄—H₂O | 1.69 | g | 0.845 | g |
| | ZnSO₄—7H₂O | 0.86 | g | 0.43 | g |
| | CuSO₄—5H₂O | 0.0025 | g | 0.00125 | g |
| 4. | 3 FN Lite Halides 100x Stock | | | | |
| | CaCl₂—2H₂O | 30.0 | g | 15.0 | g |
| 5. | KI | 0.083 | g | 0.0715 | g |
| | CoCl₂—6H₂O | 0.0025 | g | 0.00125 | g |
| 6. | 4 FN LiteP, B, Mo 100x Stock | | | | |
| | KH₂PO₄ | 18.5 | g | 9.25 | g |
| | H₃BO₃ | 0.62 | g | 0.31 | g |
| | Na₂MoO₄—2H₂O | 0.025 | g | 0.0125 | g |
| 7. | | | | | |

SB1 Solid Medium (per liter)

1 package MS salts (Gibco/BRL - Cat. No. 11117-066)
1 mL B5 vitamins 1000X stock
31.5 g Glucose
2 mL 2,4-D (20 mg/L final concentration)
pH 5.7
8 g TC agar

SB199 Solid Medium (per liter)

| | |
|---|---|
| | 1 package MS salts (Gibco/BRL - Cat. No. 11117-066) |
| | 1 mL B5 vitamins 1000X stock |
| | 30 g Sucrose |
| | 4 ml 2,4-D (40 mg/L final concentration) |
| | pH 7.0 |
| | 2 gm Gelrite |
| 8. | |

SB 166 Solid Medium (per liter)

1 package MS salts (Gibco/BRL - Cat. No. 11117-066)
1 mL B5 vitamins 1000X stock
60 g maltose
750 mg MgCl₂ hexahydrate
5 g activated charcoal
pH 5.7
2 g gelrite

SB 103 Solid Medium (per liter)

1 package MS salts (Gibco/BRL - Cat. No. 11117-066)
1 mL B5 vitamins 1000X stock
60 g maltose
750 mg MgCl2 hexahydrate
pH 5.7
2 g gelrite

SB 71-4 Solid Medium (per liter)

1 bottle Gamborg's B5 salts w/sucrose (Gibco/BRL - Cat. No. 21153-036)
pH 5.7
5 g TC agar

2,4-D Stock

Obtain premade from Phytotech Cat. No. D 295 - concentration 1 mg/mL

B5 Vitamins Stock (per 100 mL)

Store aliquots at −20° C.

10 g myo-inositol
100 mg nicotinic acid
100 mg pyridoxine HCl
1 g thiamine

If the solution does not dissolve quickly enough, apply a low level of heat via the hot stir plate.

SB 228- Soybean Histodifferentiation & Maturation (SHaM) (per liter)

| | | |
|---|---|---|
| DDI H₂O | 600 | mL |
| FN-Lite Macro Salts for SHaM 10X | 100 | mL |
| MS Micro Salts 1000x | 1 | mL |
| MS FeEDTA 100x | 10 | mL |
| CaCl 100x | 6.82 | mL |
| B5 Vitamins 1000x | 1 | mL |
| L-Methionine | 0.149 | g |
| Sucrose | 30 | g |
| Sorbitol | 30 | g |
| Adjust volume to 900 mL | | |
| pH 5.8 | | |
| Autoclave | | |
| Add to cooled media (≦30 C.): | | |
| *Glutamine (final concentration 30 mM) 4% | 110 | mL |

*Note: Final volume will be 1010 mL after glutamine addition.
Since glutamine degrades relatively rapidly, it may be preferable to add immediately prior to
using media. Expiration 2 weeks after glutamine is added; base media can be kept longer w/o
glutamine.
FN-lite Macro for SHAM 10X- Stock #1 (per liter)

| | | |
|---|---|---|
| (NH₄)2SO₄ (ammonium sulfate) | 4.63 | g |
| KNO₃ (potassium nitrate) | 28.3 | g |
| MgSO4*7H₂0 (magnesium sulfate heptahydrate) | 3.7 | g |
| KH₂PO₄ (potassium phosphate, monobasic) | 1.85 | g |
| Bring to volume | | |
| Autoclave | | |

MS Micro 1000X- Stock #2 (per 1 liter)

| | | |
|---|---|---|
| H₃BO₃ (boric acid) | 6.2 | g |
| MnSO₄*H₂O (manganese sulfate monohydrate) | 16.9 | g |
| ZnSO4*7H20 (zinc sulfate heptahydrate) | 8.6 | g |
| Na₂MoO₄*2H20 (sodium molybdate dihydrate) | 0.25 | g |
| CuSO₄*5H₂0 (copper sulfate pentahydrate) | 0.025 | g |
| CoCl₂*6H₂0 (cobalt chloride hexahydrate) | 0.025 | g |
| KI (potassium iodide) | 0.8300 | g |
| Bring to volume | | |
| Autoclave | | |

FeEDTA 100X- Stock #3 (per liter)

| | | |
|---|---|---|
| Na₂EDTA* (sodium EDTA) | 3.73 | g |
| FeSO₄*7H₂0 (iron sulfate heptahydrate) | 2.78 | g |

*EDTA must be completely dissolved before adding iron.

Bring to Volume

Solution is photosensitive. Bottle(s) should be wrapped in foil to omit light.

Autoclave

Ca 100X- Stock #4 (per liter)

| | | |
|---|---|---|
| CaCl₂*2H₂0 (calcium chloride dihydrate) | 44 | g |
| Bring to Volume | | |
| Autoclave | | |

B5 Vitamin 1000X- Stock #5 (per liter)

| | | |
|---|---|---|
| Thiamine*HCl | 10 | g |
| Nicotinic Acid | 1 | g |
| Pyridoxine*HCl | 1 | g |
| Myo-Inositol | 100 | g |
| Bring to Volume | | |
| Store frozen | | |

4% Glutamine- Stock #6 (per liter)

| | | |
|---|---|---|
| DDI water heated to 30° C. | 900 | ml |
| L-Glutamine | 40 | g |

Gradually add while stirring and applying low heat.

Do not exceed 35° C.
Bring to Volume
Filter Sterilize
Store frozen*
*Note: Warm thawed stock in 31° C. bath to fully dissolve crystals.

Regeneration of Soybean Somatic Embryos into Plants:

In order to obtain whole plants from embryogenic suspension cultures, the tissue must be regenerated. Embyros are matured as described in above. After subculturing on medium SB103 for 3 weeks, individual embryos can be removed from the clusters and screened for the desired phenotype as described in Example 1 or 2. It should be noted that any detectable phenotype, resulting from the expression of the genes of interest, could be screened at this stage.

Matured individual embryos are desiccated by placing them into an empty, small petri dish (35×10 mm) for approximately 4 to 7 days. The plates are sealed with fiber tape (creating a small humidity chamber). Desiccated embryos are planted into SB71-4 medium where they are left to germinate under the same culture conditions described above. Germinated plantlets are removed from germination medium and rinsed thoroughly with water and then are planted in Redi-Earth in 24-cell pack tray, covered with clear plastic dome. After 2 weeks the dome is removed and plants hardened off for a further week. If plantlets looked hardy they are transplanted to 10″ pot of Redi-Earth with up to 3 plantlets per pot. After 10 to 16 weeks, mature seeds are harvested, chipped and analyzed.

### Example 4

### Further Shuffling for Improved Ethametsulfuron Repressor Variants

### A. Fourth Round Shuffling

Fourth round shuffling was designed from phylogenetic alignments of TetR(B) homologues at 13 previously untested positions in addition to retesting selected substitutions at 23 previously shuffled positions. Also, the six cysteine residues aligning to wt TetR were varied with phylogenetically available diversity. This brought the total number of shuffled residues to 42. To screen this diversity two libraries, L10 and L11, were constructed (Table 5). As was done for L4 the diversity was titrated into the synthetic oligonucleotide mixture along with oligonucleotides representing parent clone L7-A11 to reduce the complexity of each individual clone (Table 6A-C).

TABLE 5
Diversity summary for libraries L10 thru L15.

| Residue | TetR(B) | | | | | |
|---|---|---|---|---|---|---|
| position | Residue | L10 | L11 | L12 | L13 | L15 |
| 55 | L | M | M | M | M | |
| 57 | I | IF | — | IF | — | |
| 60 | L | — | — | — | LF | |
| 61 | D | — | NED | — | — | |
| 62 | R | PR | — | PR | — | [symbol] |
| 64 | H | A | A | A | ADEKR | [symbol] |
| 65 | T | [symbol] | — | [symbol] | — | IT |
| 66 | H | — | HQY | — | — | |
| 67 | F | LFY | Y | [symbol] | Y | |
| 68 | C | LSC | LSC | LC | LC | |
| 69 | P | [symbol] | — | L | — | |
| 71 | E | — | VE | — | — | |
| 73 | E | [symbol] | — | AE | — | |
| 77 | D | — | DN | DNQ | — | DN |
| 82 | N | [symbol] | — | [symbol] | [symbol] | [symbol] |
| 86 | F | M | M | M | [symbol] | |
| 88 | C | RNC | RNC | N | N | |
| 99 | V | [symbol] | — | — | — | |
| 100 | H | C | C | C | [symbol] | [symbol] |
| 104 | R | G | GA | G | G | |
| 105 | P | FL | [symbol] | F | F | |
| 108 | K | Q | [symbol] | Q | Q | [symbol] |
| 109 | Q | QN | — | — | — | |
| 113 | L | AT | LVIA | A | AM | [symbol] |
| 114 | E | — | — | — | — | |
| 116 | Q | SR | MQS | SRQ | [symbol] | [symbol] |
| 121 | C | TC | [symbol] | T | T | |
| 129 | N | — | NHQ | NQ | — | |
| 134 | L | MW | M | M | [symbol] | FMNR |
| 135 | S | Q | RQ | Q | Q | |
| 136 | A | SAD | — | — | — | |
| 138 | G | — | [symbol] | — | — | |
| 139 | H | I | I | I | I | |
| 140 | F | Y | [symbol] | Y | Y | |
| 144 | C | [symbol] | [symbol] | — | — | |
| 145 | V | VA | — | — | — | |
| 147 | E | — | [symbol] | L | L | |
| 151 | H | L | [symbol] | L | L | |
| 162 | T | — | QT | — | — | |
| 166 | M | MK | — | — | — | |
| 170 | L | VI | V | V | V | |
| 174 | I | L | LVW | L | [symbol] | [symbol] |
| 175 | E | EN | — | — | — | |
| 177 | F | K | [symbol] | K | [symbol] | [symbol] |
| 183 | E | — | EDG | — | — | |
| 184 | P | PL | — | — | — | |
| 185 | A | — | AD | — | — | |
| 195 | C | SRAC | SRAC | S | S | |
| 203 | C | SRAC | [symbol] | A | A | |

(—) = same as TetR
Italic = biased incorporation by design
BOLD and Oversized = Bias from screening
Residues in parentheses = unintended mutations

TABLE 6A
Oligonucleotides for assembly and rescue of
Libraries L10 and L11.

| Oligo | SEQ | | |
|---|---|---|---|
| Name | ID No | Sequence | Pool # |
| L10:1 | 890 | TGGCACGTCAAGAACAAGCGAGCTCTGCTAGACGCTATGGCC | 10a |
| L10:2 | 891 | ATCGAGATGCTCGATCSCCACGCTATACACTWCTTACYCTTG | 10b |
| L10:3 | 892 | TTCGAGATGCTCGATCSCCACGCTATACACTWCTTACYCTTG | |
| L10:4 | 893 | ATCGAGATGCTCGATCSCCACGCTATACACTWCWGTCYCTTG | |
| L10:5 | 894 | TTCGAGATGCTCGATCSCCACGCTATACACTWCWGTCYCTTG | |
| L10:6 | 895 | ATCGAGATGCTCGATCSCCACGCTATACACTTGTTACYCTTG | |
| L10:7 | 896 | TTCGAGATGCTCGATCSCCACGCTATACACTTGTTACYCTTG | |
| L10:8 | 897 | ATCGAGATGCTCGATCSCCACGCTATACACTTGWGTCYCTTG | |
| L10:9 | 898 | TTCGAGATGCTCGATCSCCACGCTATACACTTGWGTCYCTTG | |
| L10:10 | 899 | ATCGAGATGCTCGATCSCCACGCTMCCCACTWCTTACYCTTG | |
| L10:11 | 900 | TTCGAGATGCTCGATCSCCACGCTMCCCACTWCTTACYCTTG | |
| L10:12 | 901 | ATCGAGATGCTCGATCSCCACGCTMCCCACTWCWGTCYCTTG | |
| L10:13 | 902 | TTCGAGATGCTCGATCSCCACGCTMCCCACTWCWGTCYCTTG | |
| L10:14 | 903 | ATCGAGATGCTCGATCSCCACGCTMCCCACTTGTTACYCTTG | |
| L10:15 | 904 | TTCGAGATGCTCGATCSCCACGCTMCCCACTTGTTACYCTTG | |
| L10:16 | 905 | ATCGAGATGCTCGATCSCCACGCTMCCCACTTGWGTCYCTTG | |
| L10:17 | 906 | TTCGAGATGCTCGATCSCCACGCTMCCCACTTGWGTCYCTTG | |
| L10:18 | 907 | GAAGGGGMAAGCTGGCAAGACTTCTTGAGGAACAAMGCTAAG | 10c |
| L10:19 | 908 | TCCATGAGAAACGCTTTGCTCAGTCACCGTGATGGAGCCAAG | 10d |
| L10:20 | 909 | TCCATGAGAYGTGCTTTGCTCAGTCACCGTGATGGAGCCAAG | |
| L10:21 | 910 | GCGTGTCTAGGTACGGGCTTMACGGAGCAAAACTATGAAACT | 10e |
| L10:22 | 911 | GTGTGTCTAGGTACGGGCTTMACGGAGCAAAACTATGAAACT | |
| L10:23 | 912 | GCGTGTCTAGGTACGGGCTTMACGGAGCAACAATATGAAACT | |
| L10:24 | 913 | GTGTGTCTAGGTACGGGCTTMACGGAGCAACAATATGAAACT | |
| L10:25 | 914 | ACGGAGAACMGCCTTGCCTTCCTGTGTCAACAAGGTTTCTCC | 10f |
| L10:26 | 915 | GCGGAGAACMGCCTTGCCTTCCTGTGTCAACAAGGTTTCTCC | |
| L10:27 | 916 | ACGGAGAACMGCCTTGCCTTCCTGACGCAACAAGGTTTCTCC | |
| L10:28 | 917 | GCGGAGAACMGCCTTGCCTTCCTGACGCAACAAGGTTTCTCC | |
| L10:29 | 918 | CTTGAGAACGCCCTCTACGCATGGCAAGACSTGGGGATCTAC | 10g |
| L10:30 | 919 | CTTGAGAACGCCCTCTACGCATGGCAAKCASTGGGGATCTAC | |
| L10:31 | 920 | CTTGAGAACGCCCTCTACGCAATGCAAGACSTGGGGATCTAC | |
| L10:32 | 921 | CTTGAGAACGCCCTCTACGCAATGCAAKCASTGGGGATCTAC | |
| L10:33 | 922 | ACTCTGGGTTGSGYGTTGCTGGATCAAGAGCTGCAAGTCGCT | 10h |
| L10:34 | 923 | ACTCTGGGTKCGGYGTTGCTGGATCAAGAGCTGCAAGTCGCT | |
| L10:35 | 924 | AAGGAGGAGAGGGAAACACCTACTACTGATAGTAWGCCGCCA | 10i |
| L10:36 | 925 | CTGRTACGACAAGCTCTGAACCTCAAGGATCACCAAGGTGCA | 10j |
| L10:37 | 926 | CTGRTACGACAAGCTCTGGAACTCAAGGATCACCAAGGTGCA | |
| L10:38 | 927 | GAGCYCGCCTTCCTGTTCGGCCTTGAACTGATCATAGCTGGA | 10k |
| L10:39 | 928 | GAGCYCGCCTTCCTGTTCGGCCTTGAACTGATCATAHGCGGA | |
| L10:40 | 929 | TTGGAGAAGCAGCTGAAGGCTGAAAGTGGGTCTTAATGATAG | 10L |
| L10:41 | 930 | TTGGAGAAGCAGCTGAAGHGTGAAAGTGGGTCTTAATGATAG | |
| L10:42 | 031 | GTGGSGATCGAGCATCTCGAWGGCCATAGCGTCTAGCAGAGC | 10m |
| L10:43 | 932 | GTCTTGCCAGCTTKCCCCTTCCAAGRGTAAGWAGTGTATAGC | 10n |
| L10:44 | 933 | GTCTTGCCAGCTTKCCCCTTCCAAGRGACWGWAGTGTATAGC | |
| L10:45 | 934 | GTCTTGCCAGCTTKCCCCTTCCAAGRGTAACAAGTGTATAGC | |
| L10:46 | 935 | GTCTTGCCAGCTTKCCCCTTCCAAGRGACWCAAGTGTATAGC | |
| L10:47 | 935 | GTCTTGCCAGCTTKCCCCTTCCAAGRGTAAGWAGTGGGKAGC | |
| L10:48 | 937 | GTCTTGCCAGCTTKCCCCTTCCAAGRGACWGWAGTGGGKAGC | |
| L10:49 | 938 | GTCTTGCCAGCTTKCCCCTTCCAAGRGTAACAAGTGGGKAGC | |
| L10:50 | 939 | GTCTTGCCAGCTTKCCCCTTCCAAGRGACWCAAGTGGGKAGC | |
| L10:51 | 940 | GAGCAAAGCGTTTCTCATGGACTTAGCKTTGTTCCTCAAGAA | 10o |
| L10:52 | 041 | GAGCAAAGCACRTCTCATGGACTTAGCKTTGTTCCTCAAGAA | |
| L10:53 | 942 | GAAGCCCGTACCTAGACACRCCTTGGCTCCATCACGGTGACT | 10p |
| L10:54 | 943 | TAAGCCCGTACCTAGACACRCCTTGGCTCCATCACGGTGACT | |
| L10:55 | 944 | GAAGGCAAGGCKGTTCTCCGYAGTTTCATAGTTTTGCTCCGT | 10q |
| L10:56 | 945 | GAAGGCAAGGCKGTTCTCCGYAGTTTCATATTGTTGCTCCGT | |
| L10:57 | 946 | TGCGTAGAGGGCGTTCTCAAGGGAGAAACCTTGTTGACACAG | 10r |
| L10:58 | 947 | TGCGTAGAGGGCGTTCTCAAGGGAGAAACCTTGTTGCGTCAG | |
| L10:59 | 948 | CAGCAACRCSCAACCCAGAGTGTAGATCCCCASGTCTTGCCA | 10s |
| L10:60 | 949 | CAGCAACRCCGMACCCAGAGTGTAGATCCCCASGTCTTGCCA | |
| L10:61 | 950 | CAGCAACRCSCAACCCAGAGTGTAGATCCCCASTGMTTGCCA | |
| L10:62 | 051 | CAGCAACRCCGMACCCAGAGTGTAGATCCCCASTGMTTGCCA | |
| L10:63 | 952 | CAGCAACRCSCAACCCAGAGTGTAGATCCCCASGTCTTGCAT | |
| L10:64 | 953 | CAGCAACRCCGMACCCAGAGTGTAGATCCCCASGTCTTGCAT | |
| L10:65 | 954 | CAGCAACRCSCAACCCAGAGTGTAGATCCCCASTGMTTGCAT | |
| L10:66 | 955 | CAGCAACRCCGMACCCAGAGTGTAGATCCCCASTGMTTGCAT | |
| L10:67 | 956 | AGGTGTTTCCCTCTCCTCCTTAGCGACTTGCAGCTCTTGATC | 10t |
| L10:68 | 957 | GTTCAGAGCTTGTCGTAYCAGTGGCGGCWTACTATCAGTAGT | 10u |
| L10:69 | 958 | TTCCAGAGCTTGTCGTAYCAGTGGCGGCWTACTATCAGTAGT | |
| L10:70 | 959 | GCCGAACAGGAAGGCGRGCTCTGCACCTTGGTGATCCTTGAG | 10v |
| L10:71 | 960 | AGCCTTCAGCTGCTTCTCCAATCCAGCTATGATCAGTTCAAG | 10w |
| L10:72 | 061 | ACGCTTCAGCTGCTTCTCCAATCCAGCTATGATCAGTTCAAG | |
| L10:73 | 962 | ACTCTTCAGCTGCTTCTCCAATCCAGCTATGATCAGTTCAAG | |
| L10:74 | 963 | ACACTTCAGCTGCTTCTCCAATCCAGCTATGATCAGTTCAAG | |
| L10:75 | 964 | AGCCTTCAGCTGCTTCTCCAATCCGCDTATGATCAGTTCAAG | |
| L10:76 | 965 | ACGCTTCAGCTGCTTCTCCAATCCGCDTATGATCAGTTCAAG | |
| L10:77 | 966 | ACTCTTCAGCTGCTTCTCCAATCCGCDTATGATCAGTTCAAG | |
| L10:78 | 967 | ACACTTCAGCTGCTTCTCCAATCCGCDTATGATCAGTTCAAG | |
| L10:79 | 968 | GCGCCAAGGTACCTTCTGCAGCTATCATTAAGACCCACTTTC | 10x |

TABLE 6B

| Oligo | SEQ ID | | |
|---|---|---|---|
| Name | NO | Sequence | Pool # |
| L11:1 | 969 | TGGCACGTCAAGAACAAGCGAGCTCTGCTAGACGCTATGGCC | 11a |
| L11:2 | 970 | ATTGAGATGCTCAACAGGCACGCTACCCASTACCTACCTTTG | 11b |
| L11:3 | 971 | ATTGAGATGCTCAACAGGCACGCTACCCASTACTSTCCTTTG | |
| L11:4 | 972 | ATTGAGATGCTCAACAGGCACGCTACCTATTACCTACCTTTG | |
| L11:5 | 973 | ATTGAGATGCTCAACAGGCACGCTACCTATTACTSTCCTTTG | |
| L11:6 | 974 | ATTGAGATGCTCGAKAGGCACGCTACCCASTACCTACCTTTG | |
| L11:7 | 975 | ATTGAGATGCTCGAKAGGCACGCTACCCASTACTSTCCTTTG | |
| L11:8 | 976 | ATTGAGATGCTCGAKAGGCACGCTACCTATTACCTACCTTTG | |
| L11:9 | 977 | ATTGAGATGCTCGAKAGGCACGCTACCTATTACTSTCCTTTG | |
| L11:10 | 978 | GWGGGGGAAAGCTGGCAARATTTCTTGAGGAACAACGCTAAG | 11c |
| L11:11 | 979 | TCCATGAGAAATGCTTTGCTCAGTCACCGTGATGGAGCCAAG | 11d |
| L11:12 | 980 | TCCATGAGAYGTGCTTTGCTCAGTCACCGTGATGGAGCCAAG | |
| L11:13 | 981 | GTCTGTCTAGGTACGGSGDTCACGGAGAACCAGTATGAAACT | 11e |
| L11:14 | 982 | GTCTGTCTAGGTACGGSGDTCACGGAGCAACAGTATGAAACT | |
| L11:15 | 983 | GTCTGTCTAGGTACGGSGTGGACGGAGAACCAGTATGAAACT | |
| L11:16 | 984 | GTCTGTCTAGGTACGGSGTGGACGGAGCAACAGTATGAAACT | |
| L11:17 | 985 | CTTGAGAACTCACTTGCCTTCCTGTGCCAACAAGGTTTCTCC | 11f |
| L11:18 | 986 | GTTGAGAACTCACTTGCCTTCCTGTGCCAACAAGGTTTCTCC | |
| L11:19 | 987 | ATTGAGAACTCACTTGCCTTCCTGTGCCAACAAGGTTTCTCC | |
| L11:20 | 988 | CTTGAGAACTCACTTGCCTTCCTGACGCAACAAGGTTTCTCC | |
| L11:21 | 989 | GTTGAGAACTCACTTGCCTTCCTGACGCAACAAGGTTTCTCC | |
| L11:22 | 990 | ATTGAGAACTCACTTGCCTTCCTGACGCAACAAGGTTTCTCC | |
| L11:23 | 991 | CTTGAGAACCAGCTTGCCTTCCTGTGCCAACAAGGTTTCTCC | |
| L11:24 | 992 | GTTGAGAACCAGCTTGCCTTCCTGTGCCAACAAGGTTTCTCC | |
| L11:25 | 993 | ATTGAGAACCAGCTTGCCTTCCTGTGCCAACAAGGTTTCTCC | |
| L11:26 | 994 | CTTGAGAACCAGCTTGCCTTCCTGACGCAACAAGGTTTCTCC | |
| L11:27 | 995 | GTTGAGAACCAGCTTGCCTTCCTGACGCAACAAGGTTTCTCC | |
| L11:28 | 996 | ATTGAGAACCAGCTTGCCTTCCTGACGCAACAAGGTTTCTCC | |
| L11:29 | 997 | CTTGAGAACATGCTTGCCTTCCTGTGCCAACAAGGTTTCTCC | |
| L11:30 | 998 | GTTGAGAACATGCTTGCCTTCCTGTGCCAACAAGGTTTCTCC | |
| L11:31 | 999 | ATTGAGAACATGCTTGCCTTCCTGTGCCAACAAGGTTTCTCC | |
| L11:32 | 1000 | CTTGAGAACATGCTTGCCTTCCTGACGCAACAAGGTTTCTCC | |
| L11:33 | 1001 | GTTGAGAACATGCTTGCCTTCCTGACGCAACAAGGTTTCTCC | |
| L11:34 | 1002 | ATTGAGAACATGCTTGCCTTCCTGACGCAACAAGGTTTCTCC | |
| L11:35 | 1003 | GCCGAGAACTCACTTGCCTTCCTGTGCCAACAAGGTTTCTCC | |
| L11:36 | 1004 | GCCGAGAACTCACTTGCCTTCCTGACGCAACAAGGTTTCTCC | |
| L11:37 | 1005 | GCCGAGAACCAGCTTGCCTTCCTGTGCCAACAAGGTTTCTCC | |
| L11:38 | 1006 | GCCGAGAACCAGCTTGCCTTCCTGACGCAACAAGGTTTCTCC | |
| L11:39 | 1007 | GCCGAGAACATGCTTGCCTTCCTGTGCCAACAAGGTTTCTCC | |
| L11:40 | 1008 | GCCGAGAACATGCTTGCCTTCCTGACGCAACAAGGTTTCTCC | |
| L11:41 | 1009 | CTTGAGAATGCCCTCTACGCAATGCRGGCTGTTCGGATCTWC | 11g |
| L11:42 | 1010 | CTTGAGAATGCCCTCTACGCAATGCRGGCTGTTGSCATCTWC | |
| L11:43 | 1011 | CTTGAGCAWGCCCTCTACGCAATGCRGGCTGTTCGGATCTWC | |
| L11:44 | 1012 | CTTGAGCAWGCCCTCTACGCAATGCRGGCTGTTGSCATCTWC | |
| L11:45 | 1013 | ACTCTGGGTTSCGTCTTGTGGGATCAAGAGCTACAAGTCGCT | 11h |
| L11:46 | 1014 | ACTCTGGGTTSCGTCTTGTGGGATCAAGAGADGCAAGTCGCT | |
| L11:47 | 1015 | ACTCTGGGTTSCGTCTTGSTAGATCAAGAGCTACAAGTCGCT | |
| L11:48 | 1016 | ACTCTGGGTTSCGTCTTGSTAGATCAAGAGADGCAAGTCGCT | |
| L11:49 | 1017 | AAGGAGGAGAGGGAAACACCTACTACTGATAGTATGCCGCCA | 11i |
| L11:50 | 1018 | AAGGAGGAGAGGGAAACACCTCAGACTGATAGTATGCCGCCA | |
| L11:51 | 1019 | CTGGTTCGACAAGCTKTGGAACTCCDGGATCACCAAGGTGCA | 11j |
| L11:52 | 1020 | CTGGTTCGACAAGCTKTGGAACTCAAAGATCACCAAGGTGCA | |
| L11:53 | 1021 | CTGGTTCGACAAGCTTGGGAACTCCDGGATCACCAAGGTGCA | |
| L11:54 | 1022 | CTGGTTCGACAAGCTTGGGAACTCAAAGATCACCAAGGTGCA | |
| L11:55 | 1023 | GRWCCAGMTTTCCTGTTCGGCCTTGAACTGATCATAGCAGGA | 11k |
| L11:56 | 1024 | GRWCCAGMTTTCCTGTTCGGCCTTGAACTGATCATAHGCGGA | |
| L11:57 | 1025 | TTGGAGAAGCAGCTGAAGHGCGAAAGTGGGTCTTAATGATAG | 11L |
| L11:58 | 1026 | TTGGAGAAGCAGCTGAAGGCGGAAAGTGGGTCTTAATGATAG | |
| L11:59 | 1027 | GTGCCTGTTGAGCATCTCAATGGCCATAGCGTCTAGCAGAGC | 11m |
| L11:60 | 1028 | GTGCCTMTCGAGCATCTCAATGGCCATAGCGTCTAGCAGAGC | |
| L11:61 | 1029 | ATYTTGCCAGCTTTCCCCCWCCAAAGGTAGGTASTGGGTAGC | 11n |
| L11:62 | 1030 | ATYTTGCCAGCTTTCCCCCWCCAAAGGASAGTASTGGGTAGC | |
| L11:63 | 1031 | ATYTTGCCAGCTTTCCCCCWCCAAAGGTAGGTAATAGGTAGC | |
| L11:64 | 1032 | ATYTTGCCAGCTTTCCCCCWCCAAAGGASAGTAATAGGTAGC | |
| L11:65 | 1033 | GAGCAAAGCATTTCTCATGGACTTAGCGTTGTTCCTCAAGAA | 11o |
| L11:66 | 1034 | GAGCAAAGCACRTCTCATGGACTTAGCGTTGTTCCTCAAGAA | |
| L11:67 | 1035 | GAHCSCCGTACCTAGACAGACCTTGGCTCCATCACGGTGACT | 11p |
| L11:68 | 1036 | CCACSCCGTACCTAGACAGACCTTGGCTCCATCACGGTGACT | |
| L11:69 | 1037 | GAAGGCAAGTGAGTTCTCAABAGTTTCATACTGGTTCTCCGT | 11q |
| L11:70 | 1038 | GAAGGCAAGCTGGTTCTCAABAGTTTCATACTGGTTCTCCGT | |
| L11:71 | 1039 | GAAGGCAAGCATGTTCTCAABAGTTTCATACTGGTTCTCCGT | |
| L11:72 | 1040 | GAAGGCAAGTGAGTTCTCGGCAGTTTCATACTGGTTCTCCGT | |
| L11:73 | 1041 | GAAGGCAAGCTGGTTCTCGGCAGTTTCATACTGGTTCTCCGT | |
| L11:74 | 1042 | GAAGGCAAGCATGTTCTCGGCAGTTTCATACTGGTTCTCCGT | |
| L11:75 | 1043 | GAAGGCAAGTGAGTTCTCAABAGTTTCATACTGTTGCTCCGT | |
| L11:76 | 1044 | GAAGGCAAGCTGGTTCTCAABAGTTTCATACTGTTGCTCCGT | |
| L11:77 | 1045 | GAAGGCAAGCATGTTCTCAABAGTTTCATACTGTTGCTCCGT | |
| L11:78 | 1046 | GAAGGCAAGTGAGTTCTCGGCAGTTTCATACTGTTGCTCCGT | |
| L11:79 | 1047 | GAAGGCAAGCTGGTTCTCGGCAGTTTCATACTGTTGCTCCGT | |
| L11:80 | 1048 | GAAGGCAAGCATGTTCTCGGCAGTTTCATACTGTTGCTCCGT | |
| L11:81 | 1049 | TGCGTAGAGGGCATTCTCAAGGGAGAAACCTTGTTGGCACAG | 11r |
| L11:82 | 1050 | TGCGTAGAGGGCWTGCTCAAGGGAGAAACCTTGTTGGCACAG | |
| L11:83 | 1051 | TGCGTAGAGGGCATTCTCAAGGGAGAAACCTTGTTGCGTCAG | |
| L11:84 | 1052 | TGCGTAGAGGGCWTGCTCAAGGGAGAAACCTTGTTGCGTCAG | |
| L11:85 | 1053 | CCACAAGACGSAACCCAGAGTGWAGATCCGAACAGCCYGCAT | 11s |
| L11:86 | 1054 | TASCAAGACGSAACCCAGAGTGWAGATCCGAACAGCCYGCAT | |
| L11:87 | 1055 | CCACAAGACGSAACCCAGAGTGWAGATGSCAACAGCCYGCAT | |
| L11:88 | 1056 | TASCAAGACGSAACCCAGAGTGWAGATGSCAACAGCCYGCAT | |
| L11:89 | 1057 | AGGTGTTTCCCTCTCCTCCTTAGCGACTTGTAGCTCTTGATC | 11t |
| L11:90 | 1058 | AGGTGTTTCCCTCTCCTCCTTAGCGACTTGCHTCTCTTGATC | |
| L11:91 | 1059 | TTCCAMAGCTTGTCGAACCAGTGGCGGCATACTATCAGTAGT | 11u |
| L11:92 | 1060 | TTCCCAAGCTTGTCGAACCAGTGGCGGCATACTATCAGTAGT | |
| L11:93 | 1061 | TTCCAMAGCTTGTCGAACCAGTGGCGGCATACTATCAGTCTG | |
| L11:94 | 1062 | TTCCCAAGCTTGTCGAACCAGTGGCGGCATACTATCAGTCTG | |
| L11:95 | 1063 | GCCGAACAGGAAAKCTGGWYCTGCACCTTGGTGATCCHGGAG | 11v |
| L11:96 | 1064 | GCCGAACAGGAAAKCTGGWYCTGCACCTTGGTGATCTTTGAG | |
| L11:97 | 1065 | GCDCTTCAGCTGCTTCTCCAATCCTGCTATGATCAGTTCAAG | 11w |
| L11:98 | 1066 | CGCCTTCAGCTGCTTCTCCAATCCTGCTATGATCAGTTCAAG | |
| L11:99 | 1067 | GCDCTTCAGCTGCTTCTCCAATCCGCDTATGATCAGTTCAAG | |
| L11:100 | 1068 | CGCCTTCAGCTGCTTCTCCAATCCGCDTATGATCAGTTCAAG | |
| L11:101 | 1069 | GCGCCAAGGTACCTTCTGCAGCTATCATTAAGACCCACTTTC | 11x |

TABLE 6C

| Oligo | SEQ ID | | |
|---|---|---|---|
| Name | NO | Sequence | Pool |
| EsRA11:1 | 1070 | TGGCACGTCAAGAACAAGCGAGCTCTGCTAGACGCTATGGCC | A11a |
| EsRA11:2 | 1071 | ATTGAGATGCTCGATAGGCACGCTACCCACTACTSTCCTTTG | A11b |
| EsRA11:3 | 1072 | ATTGAGATGCTCGATAGGCACGCTACCCACTACCTACCTTTG | |
| EsRA11:4 | 1073 | GAAGGGGAAAGCTGGCAAGACTTCTTGAGGAACAACGCTAAG | A11c |
| EsRA11:5 | 1074 | TCCATGAGAYGCGCTTTGCTCAGTCACCGTGATGGAGCCAAG | A11d |
| EsRA11:6 | 1075 | TCCATGAGAAATGCTTTGCTCAGTCACCGTGATGGAGCCAAG | |
| EsRA11:7 | 1076 | GTCTGTCTAGGTACGGGCTTCACGGAGCAACAGTATGAAACT | A11e |
| EsRA11:8 | 1077 | GCTGAGAACAGCCTTGCCTTCCTGACACAACAAGGTTTCTCC | A11f |
| EsRA11:9 | 1078 | GCTGAGAACAGCCTTGCCTTCCTGTGTCAACAAGGTTTCTCC | |
| EsRA11:10 | 1079 | CTTGAGAACGCCCTCTACGCAATGCAAGCTGTTGGGATCTAC | A11g |
| EsRA11:11 | 1080 | ACTCTGGGTWGTGTCTTGCTGGATCAAGAGCTGCAAGTCGCT | A11h |
| EsRA11:12 | 1081 | AAGGAGGAGAGGGAAACACCTACTACTGATAGTATGCCGCCA | A11i |
| EsRA11:13 | 1082 | CTGGTTCGACAAGCTCTGGAACTCAAGGATCACCAAGGTGCA | A11j |
| EsRA11:14 | 1083 | GAGCCAGCCTTCCTGTTCGGCCTTGAACTGATCATAGCAGGA | A11k |
| EsRA11:15 | 1084 | GAGCCAGCCTTCCTGTTCGGCCTTGAACTGATCATAHGCGGA | |
| EsRA11:16 | 1085 | TTGGAGAAGCAGCTGAAGGCCGAAAGTGGGTCTTAATGATAG | A11L |
| EsRA11:17 | 1086 | TTGGAGAAGCAGCTGAAGHGTGAAAGTGGGTCTTAATGATAG | |
| EsRA11:18 | 1087 | GTGCCTATCGAGCATCTCAATGGCCATAGCGTCTAGCAGAGC | A11m |
| EsRA11:19 | 1088 | GTCTTGCCAGCTTTCCCCTTCCAAAGGASAGTAGTGGGTAGC | A11n |
| EsRA11:20 | 1089 | GTCTTGCCAGCTTTCCCCTTCCAAAGGTAGGTAGTGGGTAGC | |
| EsRA11:21 | 1090 | GAGCAAAGCGCRTCTCATGGACTTAGCGTTGTTCCTCAAGAA | A11o |
| EsRA11:22 | 1091 | GAGCAAAGCATTTCTCATGGACTTAGCGTTGTTCCTCAAGAA | |
| EsRA11:23 | 1092 | GAAGCCCGTACCTAGACAGACCTTGGCTCCATCACGGTGACT | A11p |
| EsRA11:24 | 1093 | GAAGGCAAGGCTGTTCTCAGCAGTTTCATACTGTTGCTCCGT | A11q |
| EsRA11:25 | 1094 | TGCGTAGAGGGCGTTCTCAAGGGAGAAACCTTGTTGTGTCAG | A11r |
| EsRA11:26 | 1095 | TGCGTAGAGGGCGTTCTCAAGGGAGAAACCTTGTTGACACAG | |
| EsRA11:27 | 1096 | CAGCAAGACACWACCCAGAGTGTAGATCCCAACAGCTTGCAT | A11s |
| EsRA11:28 | 1097 | AGGTGTTTCCCTCTCCTCCTTAGCGACTTGCAGCTCTTGATC | A11t |
| EsRA11:29 | 1098 | TTCCAGAGCTTGTCGAACCAGTGGCGGCATACTATCAGTAGT | A11u |
| EsRA11:30 | 1099 | GCCGAACAGGAAGGCTGGCTCTGCACCTTGGTGATCCTTGAG | A11v |
| EsRA11:31 | 1100 | GGCCTTCAGCTGCTTCTCCAATCCTGCTATGATCAGTTCAAG | A11w |
| EsRA11:32 | 1101 | ACDCTTCAGCTGCTTCTCCAATCCTGCTATGATCAGTTCAAG | |
| EsRA11:33 | 1102 | GGCCTTCAGCTGCTTCTCCAATCCGCDTATGATCAGTTCAAG | |
| EsRA11:34 | 1103 | ACDCTTCAGCTGCTTCTCCAATCCGCDTATGATCAGTTCAAG | |
| EsRA11:35 | 1104 | GCGCCAAGGTACCTTCTGCAGCTATCATTAAGACCCACTTTC | A11x |
| SynSU5′ | 1105 | CACGTCAAGAACAAGCGAGCTCTGCTAGAC | |
| SynSU3′ | 1106 | GGAACTTCGGCGCGCCAAGGTACCTTCTGCAGCTATC | |

Following library assembly and cloning approximately 100-L10 and 130-L11 putative hits were identified from ˜20,000 repressor positive clones. The clones were re-arrayed and ranked for repressor and ligand activity by relative colony color on M9 X-gal indicator (U.S. Utility application Ser. No. 13/086,765, filed on Apr. 14, 2011 and in US Application Publication 2010-0105141, both of which are herein incorporated by reference in their entirety) plates containing 0, 1.5 and 7 ppb ethametsulfuron. All putative hits and 180 random clones from each library were sequenced and the data sets compared to create sequence activity relationships (Table 5). Library 10 results show P69L, E73A, and N82K substitutions are biased in improved clones while C144 was strongly selected over the diversity as 31 vs. 11; 31 vs. 10; 28 vs. 4; and 85 vs. 42% of the hits contained these residues compared to the randomly selected population, respectively. Although I57F was poorly incorporated in the library (none in the random population), it was found in 5% of the hit population—mostly associated with the top ligand responsive clones. Incorporation data for L11 shows that residues G104, F105, Q108, A113, Q135, G138, Y140, C144, L147, L151, and K177 were all nearly 100% conserved. The results for positions 104, 105, 135, 147, and 151 corroborate the results for the in vitro mutagenesis study showing these residues to be highly important for activity. Additionally, residues 68C and S116 were also selectively maintained over optional diversity while C121T and C203A were both preferred as 71 vs. 45 and 56 vs. 35% of the respective hits vs. random clones contained these latter changes. Top hits from libraries L10 and L11 are shown in Table 7.

### B. Fifth Round Shuffling:

One of the key and often overlooked aspects of any gene switch is maintenance of a very low level of expression in the ‘off’ state. To enhance the stringency of the in vivo repressor assay a new library vector, pVER7571, was constructed with a mutated ribosome binding site to lower the basal level of repressor produced in our assay strain and thus enhance the sensitivity of ‘leakiness’ detection. Library L12 was constructed in this new vector. Library L12 focused on reiterative shuffling of positive residue diversity from libraries L10 & L11 and (Table 5). Library L12 was constructed from thirty-two oligonucleotides (Table 8).

TABLE 8
Oligonucleotides for assembly of library L12.

| Oligo | Sequence | SEQ ID NO |
|---|---|---|
| L12:1 | TGGCACGTCAAGAACAAGCGAGCTCTGCTAGACGCTATGGCC | 1107 |
| L12:2 | ATCGAGATGCTCGATCSCCACGCTATACACTWTTTACYATTG | 1108 |
| L12:3 | TTCGAGATGCTCGATCSCCACGCTATACACTWTTTACYATTG | 1109 |
| L12:4 | ATCGAGATGCTCGATCSCCACGCTMCCCACTWTTTACYATTG | 1110 |
| L12:5 | TTCGAGATGCTCGATCSCCACGCTMCCCACTWTTTACYATTG | 1111 |
| L12:6 | GAAGGGGMAAGCTGGCAAAATTTCTTGAGGAACAAMGCTAAG | 1112 |
| L12:7 | TCCATGAGAAACGCTTTGCTCAGTCACCGTGATGGAGCCAAG | 1113 |
| L12:8 | GTCTGTCTAGGTACGGGCTTCACGGAGCAACAATATGAAACT | 1114 |
| L12:9 | GCGGAGAACCGCCTTGCCTTCCTGACACAACAAGGTTTCTCC | 1115 |
| L12:10 | CTTGAGAACGCCCTCTACGCATGGCAAGCAGTGGGGATCTAC | 1116 |
| L12:11 | CTTGAGCAGGCCCTCTACGCATGGCAAGCAGTGGGGATCTAC | 1117 |
| L12:12 | ACTCTGGGTTGTGTCTTGCTGGATCAAGAGCTGCAAGTCGCT | 1118 |
| L12:13 | AAGGAGGAGAGGGAAACACCTACTACTGATAGTATGCCGCCA | 1119 |
| L12:14 | CTGGTTCGACAAGCTKTAGAACTCAAGGATCACCAAGGTGCA | 1120 |
| L12:15 | CTGGTTCGACAAGCTTGGGAACTCAAGGATCACCAAGGTGCA | 1121 |
| L12:16 | GAGCCAGCCTTCCTGTTCGGCCTTGAACTGATCATATCAGGA | 1122 |
| L12:17 | TTGGAGAAGCAGCTGAAGGCAGAAAGTGGGTCTTAATGATAG | 1123 |
| L12:18 | GTGGSGATCGAGCATCTCGAWGGCCATAGCGTCTAGCAGAGC | 1124 |
| L12:19 | ATTTTGCCAGCTTKCCCCTTCCAATRGTAAAWAGTGTATAGC | 1125 |
| L12:20 | ATTTTGCCAGCTTKCCCCTTCCAATRGTAAAWAGTGGGKAGC | 1126 |
| L12:21 | GAGCAAAGCGTTTCTCATGGACTTAGCKTTGTTCCTCAAGAA | 1127 |
| L12:22 | GAAGCCCGTACCTAGACAGACCTTGGCTCCATCACGGTGACT | 1128 |
| L12:23 | GAAGGCAAGGCGGTTCTCCGCAGTTTCATATTGTTGCTCCGT | 1129 |
| L12:24 | TGCGTAGAGGGCGTTCTCAAGGGAGAAACCTTGTTGTGTCAG | 1130 |
| L12:25 | TGCGTAGAGGGCCTGCTCAAGGGAGAAACCTTGTTGTGTCAG | 1131 |
| L12:26 | CAGCAAGACACAACCCAGAGTGTAGATCCCCACTGCTTGCCA | 1132 |
| L12:27 | AGGTGTTTCCCTCTCCTCCTTAGCGACTTGCAGCTCTTGATC | 1133 |
| L12:28 | TTCTAMAGCTTGTCGAACCAGTGGCGGCATACTATCAGTAGT | 1134 |
| L12:29 | TTCCCAAGCTTGTCGAACCAGTGGCGGCATACTATCAGTAGT | 1135 |
| L12:30 | GCCGAACAGGAAGGCTGGCTCTGCACCTTGGTGATCCTTGAG | 1136 |
| L12:31 | TGCCTTCAGCTGCTTCTCCAATCCTGATATGATCAGTTCAAG | 1137 |
| L12:32 | GCGCCAAGGTACCTTCTGCAGCTATCATTAAGACCCACTTTC | 1138 |

Approximately 10,000 clones from library L12 were screened using the genetic plate assay with no inducer to detect leaky B-gal expression and then addition of 2 ppb ethametsulfuron plus and minus 0.002% arabinose. The latter treatment increases the stringency of induction since arabinose induces repressor production. Sixty-six putative hits were ranked for activity and their sequences determined Sequences were also determined from a population of 94 random clones and the two data sets compared. The data showed that wt TetR residues I57, R62, P69, E73, and N82 and substitutions T65I and F67Y were preferred. With the exception of E73 and N82 the preferences were modest. An alignment of the top hits from L12 is shown in Table 7.

### C. Sixth Round Shuffling:

A sixth round of shuffling using vector pVER7571 incorporated the best diversity from Rd5 shuffling (Table 5). The fully synthetic library was constructed from oligonucleotides shown in Table 9. 7,500 clones were screened by the M9 X-gal plate based assay for repression in the absence of any inducers and induction in the presence of 2 ppb Es+/−0.002% arabinose. Forty-six putative hits were re-arrayed and replica plated onto the same series of M9 X-gal assay plates. The hits were ranked for induction and repression and their sequences determined in addition to 92 randomly selected clones. Sequence analysis of the hit population show that N82, W116, and to a lesser extent Y174 were strongly selected against relative to the alternative diversity (2 vs 25; 0 vs. 41; and 9 vs. 45%, respectively). Also, within the top performing group of hits W82, F134, A177, and to a lesser degree Q108 were selected for improved activity relative to the alternative diversity at these positions. Sequences of L15 hits are shown in Table 7.

TABLE 9
Oligonucleotides for assembly of library L15.

| Oligo Name | Sequence | SEQ ID NO |
|---|---|---|
| L15:1 | TGGCACGTCAAGAACAAGCGAGCTCTGCTAGACGCTATGGCC | 1139 |
| L15:2 | ATAGAGATGCTCGATCSGCACCAAAYTCACTACTTACCCTTG | 1140 |
| L15:3 | ATAGAGATGCTCGATCSGCACAVGAYTCACTACTTACCCTTG | 1141 |
| L15:4 | GAAGGGGAAAGCTGGCAARATTTCTTGAGGAACWGGGCTAAG | 1142 |
| L15:5 | GAAGGGGAAAGCTGGCAARATTTCTTGAGGAACAAKGCTAAG | 1143 |
| L15:6 | TCCATGAGAAATGCTTTGCTCAGTCACCGTGATGGAGCCAAG | 1144 |
| L15:7 | GTCGCACTAGGTACGGGCTTCACGGAGMRACAATATGAAACT | 1145 |
| L15:8 | GTCTGTCTAGGTACGGGCTTCACGGAGMRACAATATGAAACT | 1146 |
| L15:9 | ATGGAGAACTSGCTTGCCTTCCTGACACAACAAGGTTTCTCC | 1147 |
| L15:10 | ATGGAGAACAASCTTGCCTTCCTGACACAACAAGGTTTCTCC | 1148 |
| L15:11 | CAAGAGAACTSGCTTGCCTTCCTGACACAACAAGGTTTCTCC | 1149 |
| L15:12 | CAAGAGAACAASCTTGCCTTCCTGACACAACAAGGTTTCTCC | 1150 |
| L15:13 | GCTGAGAACTSGCTTGCCTTCCTGACACAACAAGGTTTCTCC | 1151 |
| L15:14 | TCTGAGAACTSGCTTGCCTTCCTGACACAACAAGGTTTCTCC | 1152 |
| L15:15 | GCTGAGAACAASCTTGCCTTCCTGACACAACAAGGTTTCTCC | 1153 |
| L15:16 | TCTGAGAACAASCTTGCCTTCCTGACACAACAAGGTTTCTCC | 1154 |
| L15:17 | CTTGAGAACGCCCTCTACGCATTCCAAGCAGTGGGGATCTAC | 1155 |
| L15:18 | CTTGAGAACGCCCTCTACGCAAKGCAAGCAGTGGGGATCTAC | 1156 |
| L15:19 | CTTGAGAACGCCCTCTACGCAAATCAAGCAGTGGGGATCTAC | 1157 |
| L15:20 | ACTCTGGGTTGTGTCTTGCTGGATCAAGAGCTGCAAGTCGCT | 1158 |
| L15:21 | AAGGAGGAGAGGGAAACACCTACTACTGATAGTATGCCGCCA | 1159 |
| L15:22 | CTGGTTCGACAAGCTTACGAACTCGCGGATCACCAAGGTGCA | 1160 |
| L15:23 | CTGGTTCGACAAGCTTACGAACTCTYCGATCACCAAGGTGCA | 1161 |
| L15:24 | CTGGTTCGACAAGCTTACGAACTCAATGATCACCAAGGTGCA | 1162 |
| L15:25 | CTGGTTCGACAAGCTDTTGAACTCGCGGATCACCAAGGTGCA | 1163 |
| L15:26 | CTGGTTCGACAAGCTDTTGAACTCTYCGATCACCAAGGTGCA | 1164 |
| L15:27 | CTGGTTCGACAAGCTDTTGAACTCAATGATCACCAAGGTGCA | 1165 |
| L15:28 | GAGCCAGCCTTCCTGTTCGGCCTTGAACTGATCATATCAGGA | 1166 |
| L15:29 | TTGGAGAAGCAGCTGAAGGCCGAAAGTGGGTCTTAATGATAG | 1167 |
| L15:30 | GTGCSGATCGAGCATCTCTATGGCCATAGCGTCTAGCAGAGC | 1168 |
| L15:31 | ATYTTGCCAGCTTTCCCCTTCCAAGGGTAAGTAGTGARTTTG | 1169 |
| L15:32 | ATYTTGCCAGCTTTCCCCTTCCAAGGGTAAGTAGTGARTCBT | 1170 |
| L15:33 | GAGCAAAGCATTTCTCATGGACTTAGCCCWGTTCCTCAAGAA | 1171 |
| L15:34 | GAGCAAAGCATTTCTCATGGACTTAGCMTTGTTCCTCAAGAA | 1172 |
| L15:35 | GAAGCCCGTACCTAGTGCGACCTTGGCTCCATCACGGTGACT | 1173 |
| L15:36 | GAAGCCCGTACCTAGACAGACCTTGGCTCCATCACGGTGACT | 1174 |
| L15:37 | GAAGGCAAGCSAGTTCTCCATAGTTTCATATTGTYKCTCCGT | 1175 |
| L15:38 | GAAGGCAAGSTTGTTCTCCATAGTTTCATATTGTYKCTCCGT | 1176 |
| L15:39 | GAAGGCAAGCSAGTTCTCTTGAGTTTCATATTGTYKCTCCGT | 1177 |
| L15:40 | GAAGGCAAGSTTGTTCTCTTGAGTTTCATATTGTYKCTCCGT | 1178 |
| L15:41 | GAAGGCAAGCSAGTTCTCAGMAGTTTCATATTGTYKCTCCGT | 1179 |
| L15:42 | GAAGGCAAGSTTGTTCTCAGMAGTTTCATATTGTYKCTCCGT | 1180 |
| L15:43 | TGCGTAGAGGGCGTTCTCAAGGGAGAAACCTTGTTGTGTCAG | 1181 |
| L15:44 | CAGCAAGACACAACCCAGAGTGTAGATCCCCACTGCTTGGAA | 1182 |
| L15:45 | CAGCAAGACACAACCCAGAGTGTAGATCCCCACTGCTTGCMT | 1183 |
| L15:46 | CAGCAAGACACAACCCAGAGTGTAGATCCCCACTGCTTGATT | 1184 |
| L15:47 | AGGTGTTTCCCTCTCCTCCTTAGCGACTTGCAGCTCTTGATC | 1185 |
| L15:48 | TTCGTAAGCTTGTCGAACCAGTGGCGGCATACTATCAGTAGT | 1186 |
| L15:49 | TTCAAHAGCTTGTCGAACCAGTGGCGGCATACTATCAGTAGT | 1187 |
| L15:50 | GCCGAACAGGAAGGCTGGCTCTGCACCTTGGTGATCCGCGAG | 1188 |
| L15:51 | GCCGAACAGGAAGGCTGGCTCTGCACCTTGGTGATCGRAGAG | 1189 |
| L15:52 | GCCGAACAGGAAGGCTGGCTCTGCACCTTGGTGATCATTGAG | 1190 |
| L15:53 | GGCCTTCAGCTGCTTCTCCAATCCTGATATGATCAGTTCAAG | 1191 |
| L15:54 | GCGCCAAGGTACCTTCTGCAGCTATCATTAAGACCCACTTTC | 1192 |

TABLE 7
Sequence summary of top hits from Libraries L10, L11, L12, L13, and L15.

| | Sequence Position/Residue Substitution | | | | | | | | | | | | | | | | | | | | |
|---|---|---|---|---|---|---|---|---|---|---|---|---|---|---|---|---|---|---|---|---|---|
| Clone | 55 | 57 | 60 | 61 | 62 | 64 | 65 | 67 | 68 | 69 | 72 | 73 | 75 | 77 | 82 | 85 | 86 | 88 | 92 | 99 | 100 |
| TetR (B) | L | I | L | D | R | H | T | F | C | P | G | E | W | D | N | S | F | C | S | V | H |
| L10-A04 | M | — | — | — | — | A | — | Y | — | — | — | — | — | — | K | — | M | N | — | — | C |
| L10-A05 | M | — | — | — | — | A | — | Y | — | — | — | — | — | — | K | — | M | — | — | — | C |
| L10-A06 | M | — | — | — | — | A | — | Y | L | — | — | A | — | — | K | — | M | N | — | — | C |
| L10-A09 | M | — | — | — | P | A | I | L | L | — | — | A | — | — | — | — | M | R | — | — | C |
| L10-A11 | M | — | — | — | — | A | — | Y | L | — | — | — | — | — | K | — | M | — | — | — | C |
| L10-B02 | M | — | — | — | P | A | — | — | — | L | — | — | — | — | — | — | M | N | — | — | C |
| L10-B03 | M | — | — | — | — | A | — | Y | S | — | — | — | — | — | K | — | M | — | — | — | C |
| L10-B06 | M | — | — | — | P | A | P | L | L | — | — | A | — | — | — | — | M | — | — | — | C |
| L10-B07 | M | — | — | — | — | A | I | L | L | — | — | — | — | — | — | — | M | — | — | — | C |
| L10-B08 | M | — | — | — | — | A | — | Y | L | — | — | A | — | — | K | — | M | R | — | — | C |
| L11-C02 | M | — | — | — | P | A | — | Y | S | — | — | — | — | — | K | — | M | — | — | — | C |
| L11-C06 | M | — | — | — | — | A | — | Y | S | — | R | — | — | N | — | — | M | N | — | — | C |
| L12-1-10 | M | F | — | — | — | A | I | — | L | — | — | A | — | N | T | — | M | N | — | — | C |
| L12-1-11 | M | F | — | — | P | A | I | Y | L | — | — | — | — | N | H | — | M | N | — | — | C |
| L12-1-21 | M | F | — | — | — | A | P | Y | L | — | — | A | — | N | — | — | M | N | — | — | C |
| L12-2-13 | M | — | — | — | — | A | I | Y | L | — | — | A | — | N | — | — | M | N | — | — | C |
| L12-2-23 | M | F | — | — | — | A | — | Y | L | — | — | — | — | N | — | — | M | N | — | — | C |
| L12-2-27 | M | F | — | — | — | A | I | Y | L | — | — | A | — | N | — | — | M | N | — | L | C |
| L12-2-48 | M | — | — | — | — | A | I | Y | L | — | — | — | — | N | — | — | M | N | — | — | C |
| L13-1-9 | M | — | — | — | — | A | — | Y | — | — | — | — | — | — | K | — | M | N | — | — | A |
| L13-1-10 | M | — | F | — | — | D | — | Y | — | — | — | — | C | — | K | — | M | N | — | — | A |
| L13-1-16 | M | — | F | — | — | K | — | Y | — | — | — | — | — | — | R | — | M | N | — | — | A |
| L13-1-42 | M | — | — | — | — | K | — | Y | — | — | — | — | — | — | K | — | M | N | — | — | A |
| L13-1-43 | M | — | — | — | — | A | — | Y | — | — | — | — | — | — | R | — | M | N | — | — | A |
| L13-2-18 | M | — | F | — | — | A | — | Y | — | — | — | — | — | — | K | — | M | N | R | — | A |
| L13-2-23 | M | — | F | — | — | A | — | Y | — | — | — | — | — | — | K | — | M | N | — | — | A |
| L13-2-24 | M | — | — | — | — | K | — | Y | L | — | — | — | — | — | — | — | M | N | — | — | C |
| L15-1 | M | — | — | — | — | Q | V | N | L | — | — | — | — | — | W | — | M | N | — | — | C |
| L15-14 | M | — | — | — | — | R | I | Y | L | — | — | — | — | — | K | — | M | N | — | — | C |
| L15-20 | M | — | — | — | P | K | I | Y | L | — | — | — | — | — | R | — | M | N | — | — | C |
| L15-35 | M | — | — | — | — | T | — | Y | L | — | — | — | — | N | W | — | M | N | — | — | C |
| L15-36 | M | — | — | G | — | K | — | Y | L | — | — | — | — | — | W | — | M | N | — | — | C |
| L15-41 | M | — | — | — | — | K | — | Y | L | — | — | — | — | — | K | — | M | I | — | — | C |

| | Sequence Position/Residue Substitution | | | | | | | | | | | | | |
|---|---|---|---|---|---|---|---|---|---|---|---|---|---|---|
| Clone | 101 | 104 | 105 | 108 | 113 | 116 | 118 | 121 | 129 | 134 | 135 | 139 | 140 | 144 |
| TetR (B) | L | R | P | K | L | Q | A | C | N | L | S | H | F | C |
| L10-A04 | — | G | F | Q | A | S | — | — | — | M | Q | I | Y | — |
| L10-A05 | — | G | F | Q | A | S | — | — | — | M | Q | I | Y | — |
| L10-A06 | — | G | F | Q | A | S | — | — | — | M | Q | I | Y | — |
| L10-A09 | — | G | F | Q | A | S | — | — | — | M | Q | I | Y | — |
| L10-A11 | — | G | F | Q | A | S | — | T | — | M | Q | I | Y | — |
| L10-B02 | — | G | F | Q | A | S | — | — | — | M | Q | I | Y | — |
| L10-B03 | — | G | F | Q | A | S | — | T | — | M | Q | I | Y | — |
| L10-B06 | — | G | F | Q | A | S | — | T | — | M | Q | I | Y | — |
| L10-B07 | — | G | F | Q | A | R | — | T | — | W | Q | I | Y | — |
| L10-B08 | — | G | F | Q | A | S | — | T | — | M | Q | I | Y | — |
| L11-C02 | — | G | F | Q | A | S | — | — | — | M | Q | I | Y | S |
| L11-C06 | — | G | F | Q | A | S | — | T | — | M | Q | I | Y | — |
| L12-1-10 | — | G | F | R | A | R | — | T | Q | W | Q | I | Y | — |
| L12-1-11 | — | G | F | Q | A | R | — | T | — | W | Q | I | Y | — |
| L12-1-21 | — | G | F | Q | A | H | — | T | — | W | Q | I | Y | — |
| L12-2-13 | — | G | F | Q | A | S | — | T | Q | W | Q | I | Y | — |
| L12-2-23 | — | G | F | R | A | R | — | T | — | W | Q | I | Y | — |
| L12-2-27 | — | G | F | Q | A | R | — | T | Q | W | Q | I | Y | — |
| L12-2-48 | — | G | F | Q | A | R | — | T | — | W | Q | I | Y | — |
| L13-1-9 | — | G | F | Q | M | S | — | T | — | F | Q | I | Y | — |
| L13-1-10 | — | G | F | Q | A | S | — | T | — | F | Q | I | Y | — |
| L13-1-16 | — | G | F | Q | M | S | — | T | — | M | Q | I | Y | — |
| L13-1-42 | — | G | F | Q | M | S | — | T | — | M | Q | I | Y | — |
| L13-1-43 | — | G | F | Q | M | S | — | T | — | F | Q | I | Y | — |
| L13-2-18 | — | G | F | Q | A | C | — | T | — | F | Q | I | Y | — |
| L13-2-23 | — | G | F | Q | A | C | — | T | — | F | Q | I | Y | — |
| L13-2-24 | — | G | F | Q | A | W | — | T | — | F | Q | I | Y | — |
| L15-1 | — | G | F | R | S | K | — | T | — | F | Q | I | Y | — |
| L15-14 | — | G | F | Q | Q | S | — | T | — | N | Q | I | Y | — |
| L15-20 | — | G | F | R | A | T | — | T | — | F | Q | I | Y | — |
| L15-35 | — | G | F | Q | M | S | — | T | — | M | Q | I | Y | — |
| L15-36 | — | G | F | Q | M | N | D | T | — | M | Q | I | Y | R |
| L15-41 | — | G | F | — | A | T | — | T | — | F | Q | I | Y | — |

| | Sequence Position/Residue Substitution | | | | | | | | | | | |
|---|---|---|---|---|---|---|---|---|---|---|---|---|
| Clone | 145 | 147 | 148 | 150 | 151 | 153 | 170 | 174 | 177 | 184 | 195 | 203 |
| TetR (B) | V | E | D | E | H | V | L | I | F | P | C | C |
| L10-A04 | — | L | — | — | L | — | V | L | K | — | A | A |
| L10-A05 | — | L | — | — | L | — | V | L | K | — | S | A |
| L10-A06 | — | L | — | — | L | — | V | L | K | — | A | S |
| L10-A09 | — | L | — | — | L | — | V | L | K | — | — | A |
| L10-A11 | — | L | — | — | L | — | V | L | K | L | R | — |
| L10-B02 | — | L | — | — | L | — | V | L | K | — | S | — |
| L10-B03 | — | L | — | — | L | — | V | L | K | — | A | A |
| L10-B06 | A | L | — | — | L | — | V | L | K | — | S | R |
| L10-B07 | — | L | — | — | L | — | V | L | K | — | G | S |
| L10-B08 | — | L | N | — | L | — | V | L | K | — | A | A |
| L11-C02 | A | L | — | — | L | — | V | L | K | — | R | — |
| L11-C06 | — | L | — | — | L | — | V | V | K | — | A | — |
| L12-1-10 | — | L | — | — | L | — | V | W | K | — | S | A |
| L12-1-11 | — | L | — | — | L | — | V | W | K | — | S | A |
| L12-1-21 | — | L | — | Q | L | — | V | W | K | — | S | A |
| L12-2-13 | — | L | — | — | L | F | V | V | K | — | S | A |
| L12-2-23 | — | L | — | — | L | — | V | W | K | — | S | A |
| L12-2-27 | — | L | — | — | L | — | V | W | K | — | S | A |
| L12-2-48 | — | L | — | — | L | — | V | L | K | — | S | A |
| L13-1-9 | — | L | — | — | L | — | V | Y | K | — | — | A |
| L13-1-10 | — | L | — | — | L | — | V | — | H | — | S | — |
| L13-1-16 | — | L | — | — | L | — | V | Y | K | — | — | A |
| L13-1-42 | — | L | — | — | L | — | V | Y | K | — | S | — |
| L13-1-43 | — | L | — | — | L | — | V | Y | K | — | — | — |
| L13-2-18 | — | L | — | — | L | — | V | — | K | — | — | — |
| L13-2-23 | — | L | — | — | L | — | V | Y | K | — | — | — |
| L13-2-24 | — | L | — | — | L | — | V | L | H | — | S | A |
| L15-1 | — | L | — | — | L | — | V | — | A | — | S | A |
| L15-14 | — | L | — | — | L | — | V | — | A | — | S | A |
| L15-20 | — | L | — | — | L | — | V | Y | A | — | S | A |
| L15-35 | — | L | — | — | L | — | V | V | A | — | S | A |
| L15-36 | — | L | — | — | L | — | V | F | — | — | S | A |
| L15-41 | — | L | — | — | L | — | V | F | A | — | S | A |

Various nucleotide sequences of the top hits from Libraries L10, L11, L12, L13, and L15 are set forth in SEQ ID NOS: 1193-1380. Various amino acid sequences of top hits from Libraries L10, L11, L12, L13, and L15 are set forth in SEQ ID NOS: 1381-1568.

### Example 5

### Chlorsulfuron Repressor Shuffling

### A. Second-Round Shuffling

The original library was designed to thifensulfuron, but once induction activity was established with other SU compounds having potentially better soil and in planta stability properties than the original ligand, the evolution process was re-directed towards these alternative ligands. Of particular interest were herbicides metsulfuron, sulfometuron, ethametsulfuron and chlorsulfuron. For this objective, parental clones L1-9, -22, -29 and -44 were chosen for further shuffling. Clone L1-9 has strong activity on both ethametsulfuron and chlorsulfuron; clone L1-22 has strong sulfometuron activity; clone L1-29 has moderate metsulfuron activity; and clone L1-44 has moderate activity on metsulfuron, ethametsulfuron and chlorsulfuron. (Data not shown.). No clones found in the initial screen were exceptionally reactive to metsulfuron. These four clones were also chosen due to their relatively strong repressor activity, showing low β-gal background activity without inducer. Strong repressor activity is important for establishing a system which is both highly sensitive to the presence of inducer, and tightly off in the absence of inducer.

Based on the sequence information from parental clones L1-9, -22, -29 and -44, two second round libraries were designed, constructed and screened. The first library, L2, consisted of a ‘family’ shuffle whereby the amino acid diversity between the selected parental clones was varied using synthetic assembly of oligonucleotides to find clones improved in responsiveness to any of the four new target ligands. A summary of the diversity used and the resulting hit sequences for library L2 is shown in Table 10.

TABLE 10

| | Amino acid residue position | | | | | | | | | | |
|---|---|---|---|---|---|---|---|---|---|---|---|
| Clone | 60 | 64 | 82 | 86 | 100 | 104 | 105 | 113 | 116 | 134 | 135 |
| wt | L | H | N | F | H | R | P | L | Q | L | S |
| Parents | | | | | | | | | | | |
| L1-9 | — | A | — | M | C | G | F | A | S | M | Q |
| L1-22 | M | — | T | Y | C | A | I | K | N | R | Q |
| L1-29 | M | Q | T | M | W | — | W | P | M | W | — |
| L1-44 | — | A | — | Y | Y | A | V | A | — | V | K |
| Hits | | | | | | | | | | | |
| L2-2 | — | Q | — | M | C | — | F | K | — | V | — |
| L2-9 | M | Q | — | M | Y | — | W | A | — | W | — |
| L2-10 | — | A | — | M | W | G | W | K | M | M | — |
| L2-13 | — | Q | — | M | C | — | W | A | — | W | Q |
| L2-14 | M | A | — | M | C | — | W | A | M | V | — |
| L2-18 | M | Q | T | M | W | — | W | A | — | M | — |
| L1-45 | A | Q | — | W | W | G | L | P | V | T | Q |
| Unselected | random | random | random | random | W > C, Y | R >> G, A | W > V > | random | random | random | S >> Q, K |
| frequency | | | | | | | I, F | | | | |

| | Amino acid residue position | | | | | | | | Inducer |
|---|---|---|---|---|---|---|---|---|---|
| Clone | 138 | 139 | 147 | 151 | 164 | 174 | 177 | 203 | preference |
| wt | G | H | E | H | D | I | F | C | atc |
| Parents | | | | | | | | | |
| L1-9 | C | I | L | L | — | L | K | — | 4, 9 (weak) |
| L1-22 | R | V | F | M | — | S | L | S | 3 |
| L1-29 | C | N | S | R | — | W | S | — | 9 (weak) |
| L1-44 | A | G | W | S | A | V | A | — | 9 (weak) |
| Hits | | | | | | | | | |
| L2-2 | R | I | W | M | — | W | L | — | 4 (inverse) |
| L2-9 | A | I | W | S | — | S | K | — | 9 (leaky) |
| L2-10 | R | I | L | L | — | W | K | — | 4 (leaky) |
| L2-13 | R | I | S | M | — | V | K | — | 9 |
| L2-14 | R | V | F | S | A | L | K | — | 9 |
| L2-18 | R | N | F | L | A | W | K | — | 9 |
| L1-45 | R | — | G | R | — | A | L | — | 3, 4 |
| Unselected | A >> C, R | G, N > V > I | random | random | random | random | random | C >> S | |
| frequency | | | | | | | | | |

The oligonucleotides used to construct the library are shown in Table 11. The L2 oligonucleotides were assembled, cloned and screened as per the protocol described for library L1 except that each ligand was tested at 2 ppm to increase the stringency of the assay, which is a 10-fold reduction from 1st round library screening concentration.

TABLE 11

| | | SEQ |
|---|---|---|
| Oligo | Sequence | ID |
| L2:01 | TATTGGCATGTAAAAAATAAGCGAGCTCTGCTCGACGCC | 1569 |
| | TTA | |
| L2:02 | GCCATTGAGATGWTGGATAGGCACCASACTCACTTTTGC | 1570 |
| | CCT | |
| L2:03 | GCCATTGAGATGWTGGATAGGCACGCAACTCACTTTTGC | 1571 |
| | CCT | |
| L2:04 | TTAGAAGGGGAAAGCTGGCAAGATTTTTTACGTAATAMT | 1572 |
| | GCT | |
| L2:05 | AAAAGTTACAGATGTGCTTTACTAAGTCATCGCGATGGA | 1573 |
| | GCA | |
| L2:06 | AAAAGTATGAGATGTGCTTTACTAAGTCATCGCGATGGA | 1574 |
| | GCA | |
| L2:07 | AAAGTATRTTTAGGTACACGCDTCACAGAAAAACAGTAT | 1575 |
| | GAA | |
| L2:08 | AAAGTATRTTTAGGTACACGCTGGACAGAAAAACAGTAT | 1576 |
| | GAA | |
| L2:09 | AAAGTATRTTTAGGTACAGSTDTCACAGAAAAACAGTAT | 1577 |
| | GAA | |
| L2:10 | AAAGTATRTTTAGGTACAGSTTGGACAGAAAAACAGTAT | 1578 |
| | GAA | |
| L2:11 | AAAGTATGGTTAGGTACACGCDTCACAGAAAAACAGTAT | 1579 |
| | GAA | |
| L2:12 | AAAGTATGGTTAGGTACACGCTGGACAGAAAAACAGTAT | 1580 |
| | GAA | |
| L2:13 | AAAGTATGGTTAGGTACAGSTDTCACAGAAAAACAGTAT | 1581 |
| | GAA | |
| L2:14 | AAAGTATGGTTAGGTACAGSTTGGACAGAAAAACAGTAT | 1582 |
| | GAA | |
| L2:15 | ACTAAAGAAAATARCTTAGCCTTTTTATGCCAACAAGGT | 1583 |
| | TTT | |
| L2:16 | ACTAAAGAAAATCAATTAGCCTTTTTATGCCAACAAGGT | 1584 |
| | TTT | |
| L2:17 | ACTAAAGAAAATATGTTAGCCTTTTTATGCCAACAAGGT | 1585 |
| | TTT | |
| L2:18 | ACTSCTGAAAATARCTTAGCCTTTTTATGCCAACAAGGT | 1586 |
| | TTT | |
| L2:19 | ACTSCTGAAAATCAATTAGCCTTTTTATGCCAACAAGGT | 1587 |
| | TTT | |
| L2:20 | ACTSCTGAAAATATGTTAGCCTTTTTATGCCAACAAGGT | 1588 |
| | TTT | |
| L2:21 | TCACTAGAGAATGCATTATATGCARTGAGTGCTGTGGCT | 1589 |
| | AWT | |
| L2:22 | TCACTAGAGAATGCATTATATGCARTGAGTGCTGTGGCT | 1590 |
| | GKT | |
| L2:23 | TCACTAGAGAATGCATTATATGCARTGAGTGCTGTGYGC | 1591 |
| | AWT | |
| L2:24 | TCACTAGAGAATGCATTATATGCARTGAGTGCTGTGYGC | 1592 |
| | GKT | |
| L2:25 | TCACTAGAGAATGCATTATATGCARTGMAAGCTGTGGCT | 1593 |
| | AWT | |
| L2:26 | TCACTAGAGAATGCATTATATGCARTGMAAGCTGTGGCT | 1594 |
| | GKT | |
| L2:27 | TCACTAGAGAATGCATTATATGCARTGMAAGCTGTGYGC | 1595 |
| | AWT | |
| L2:28 | TCACTAGAGAATGCATTATATGCARTGMAAGCTGTGYGC | 1596 |
| | GKT | |
| L2:29 | TCACTAGAGAATGCATTATATGCAWGGAGTGCTGTGGCT | 1597 |
| | AWT | |
| L2:30 | TCACTAGAGAATGCATTATATGCAWGGAGTGCTGTGGCT | 1598 |
| | GKT | |
| L2:31 | TCACTAGAGAATGCATTATATGCAWGGAGTGCTGTGYGC | 1599 |
| | AWT | |
| L2:32 | TCACTAGAGAATGCATTATATGCAWGGAGTGCTGTGYGC | 1600 |
| | GKT | |
| L2:33 | TCACTAGAGAATGCATTATATGCAWGGMAAGCTGTGGCT | 1601 |
| | AWT | |
| L2:34 | TCACTAGAGAATGCATTATATGCAWGGMAAGCTGTGGCT | 1602 |
| | GKT | |
| L2:35 | TCACTAGAGAATGCATTATATGCAWGGMAAGCTGTGYGC | 1603 |
| | AWT | |
| L2:36 | TCACTAGAGAATGCATTATATGCAWGGMAAGCTGTGYGC | 1604 |
| | GKT | |
| L2:37 | TTTACTTTAGGTTGCGTATTGTKGGATCAAGAGAGMCAA | 1605 |
| | GTC | |
| L2:38 | TTTACTTTAGGTTGCGTATTGTKGGATCAAGAGMTGCAA | 1606 |
| | GTC | |
| L2:39 | TTTACTTTAGGTTGCGTATTGTYTGATCAAGAGAGMCAA | 1607 |
| | GTC | |
| L2:40 | TTTACTTTAGGTTGCGTATTGTYTGATCAAGAGMTGCAA | 1608 |
| | GTC | |
| L2:41 | GCTAAAGAAGAAAGGGAAACACCTACTACTGMTAGTATG | 1609 |
| | CCG | |
| L2:42 | CCATTATTACGACAAGCTAGTGAATTATTGGATCACCAA | 1610 |
| | GGT | |
| L2:43 | CCATTATTACGACAAGCTAGTGAATTAKCAGATCACCAA | 1611 |
| | GGT | |
| L2:44 | CCATTATTACGACAAGCTAGTGAATTAAAGGATCACCAA | 1612 |
| | GGT | |
| L2:45 | CCATTATTACGACAAGCTTKGGAATTATTGGATCACCAA | 1613 |
| | GGT | |
| L2:46 | CCATTATTACGACAAGCTTKGGAATTAKCAGATCACCAA | 1614 |
| | GGT | |
| L2:47 | CCATTATTACGACAAGCTTKGGAATTAAAGGATCACCAA | 1615 |
| | GGT | |
| L2:48 | CCATTATTACGACAAGCTGTAGAATTATTGGATCACCAA | 1616 |
| | GGT | |
| L2:49 | CCATTATTACGACAAGCTGTAGAATTAKCAGATCACCAA | 1617 |
| | GGT | |
| L2:50 | CCATTATTACGACAAGCTGTAGAATTAAAGGATCACCAA | 1618 |
| | GGT | |
| L2:51 | GCAGAGCCAGCCTTCTTATTCGGCCTTGAATTGATCATA | 1619 |
| | TGC | |
| L2:52 | GGATTAGAAAAACAACTTAAATSCGAAAGTGGGTCTTAA | 1620 |
| L2:53 | CCTATCCAWCATCTCAATGGCTAAGGCGTCGAGCAGAGC | 1621 |
| | TCG | |
| L2:54 | TTGCCAGCTTTCCCCTTCTAAAGGGCAAAAGTGAGTSTG | 1622 |
| | GTG | |
| L2:55 | TTGCCAGCTTTCCCCTTCTAAAGGGCAAAAGTGAGTTGC | 1623 |
| | GTG | |
| L2:56 | TAAAGCACATCTGTAACTTTTAGCAKTATTACGTAAAAA | 1624 |
| | ATC | |
| L2:57 | TAAAGCACATCTCATACTTTTAGCAKTATTACGTAAAAA | 1625 |
| | ATC | |
| L2:58 | GCGTGTACCTAAAYATACTTTTGCTCCATCGCGATGACT | 1626 |
| | TAG | |
| L2:59 | ASCTGTACCTAAAYATACTTTTGCTCCATCGCGATGACT | 1627 |
| | TAG | |
| L2:60 | GCGTGTACCTAACCATACTTTTGCTCCATCGCGATGACT | 1628 |
| | TAG | |
| L2:61 | ASCTGTACCTAACCATACTTTTGCTCCATCGCGATGACT | 1629 |
| | TAG | |
| L2:62 | GGCTAAGYTATTTTCTTTAGTTTCATACTGTTTTTCTGT | 1630 |
| | GAH | |
| L2:63 | GGCTAATTGATTTTCTTTAGTTTCATACTGTTTTTCTGT | 1631 |
| | GAH | |
| L2:64 | GGCTAACATATTTTCTTTAGTTTCATACTGTTTTTCTGT | 1632 |
| | GAH | |
| L2:65 | GGCTAAGYTATTTTCAGSAGTTTCATACTGTTTTTCTGT | 1633 |
| | GAH | |
| L2:66 | GGCTAATTGATTTTCAGSAGTTTCATACTGTTTTTCTGT | 1634 |
| | GAH | |
| L2:67 | GGCTAACATATTTTCAGSAGTTTCATACTGTTTTTCTGT | 1635 |
| | GAH | |
| L2:68 | GGCTAAGYTATTTTCTTTAGTTTCATACTGTTTTTCTGT | 1636 |
| | CCA | |
| L2:69 | GGCTAATTGATTTTCTTTAGTTTCATACTGTTTTTCTGT | 1637 |
| | CCA | |
| L2:70 | GGCTAACATATTTTCTTTAGTTTCATACTGTTTTTCTGT | 1638 |
| | CCA | |
| L2:71 | GGCTAAGYTATTTTCAGSAGTTTCATACTGTTTTTCTGT | 1639 |
| | CCA | |
| L2:72 | GGCTAATTGATTTTCAGSAGTTTCATACTGTTTTTCTGT | 1640 |
| | CCA | |
| L2:73 | GGCTAACATATTTTCAGSAGTTTCATACTGTTTTTCTGT | 1641 |
| | CCA | |
| L2:74 | ATATAATGCATTCTCTAGTGAAAAACCTTGTTGGCATAA | 1642 |
| | AAA | |
| L2:75 | CAATACGCAACCTAAAGTAAAAWTAGCCACAGCACTCAY | 1643 |
| | TGC | |
| L2:76 | CAATACGCAACCTAAAGTAAAAMCAGCCACAGCACTCAY | 1644 |
| | TGC | |
| L2:77 | CAATACGCAACCTAAAGTAAAAWTGCRCACAGCACTCAY | 1645 |
| | TGC | |
| L2:78 | CAATACGCAACCTAAAGTAAAAMCGCRCACAGCACTCAY | 1646 |
| | TGC | |
| L2:79 | CAATACGCAACCTAAAGTAAAAWTAGCCACAGCTTKCAY | 1647 |
| | TGC | |
| L2:80 | CAATACGCAACCTAAAGTAAAAMCAGCCACAGCTTKCAY | 1648 |
| | TGC | |
| L2:81 | CAATACGCAACCTAAAGTAAAAWTGCRCACAGCTTKCAY | 1649 |
| | TGC | |
| L2:82 | CAATACGCAACCTAAAGTAAAAMCGCRCACAGCTTKCAY | 1650 |
| | TGC | |
| L2:83 | CAATACGCAACCTAAAGTAAAAWTAGCCACAGCACTCCW | 1651 |
| | TGC | |
| L2:84 | CAATACGCAACCTAAAGTAAAAMCAGCCACAGCACTCCW | 1652 |
| | TGC | |
| L2:85 | CAATACGCAACCTAAAGTAAAAWTGCRCACAGCACTCCW | 1653 |
| | TGC | |
| L2:86 | CAATACGCAACCTAAAGTAAAAMCGCRCACAGCACTCCW | 1654 |
| | TGC | |
| L2:87 | CAATACGCAACCTAAAGTAAAAWTAGCCACAGCTTKCCW | 1655 |
| | TGC | |
| L2:88 | CAATACGCAACCTAAAGTAAAAMCAGCCACAGCTTKCCW | 1656 |
| | TGC | |
| L2:89 | CAATACGCAACCTAAAGTAAAAWTGCRCACAGCTTKCCW | 1657 |
| | TGC | |
| L2:90 | CAATACGCAACCTAAAGTAAAAMCGCRCACAGCTTKCCW | 1658 |
| | TGC | |
| L2:91 | TGTTTCCCTTTCTTCTTTAGCGACTTGKCTCTCTTGATC | 1659 |
| | CMA | |
| L2:92 | TGTTTCCCTTTCTTCTTTAGCGACTTGCAKCTCTTGATC | 1660 |
| | CMA | |
| L2:93 | TGTTTCCCTTTCTTCTTTAGCGACTTGKCTCTCTTGATC | 1661 |
| | ARA | |
| L2:94 | TGTTTCCCTTTCTTCTTTAGCGACTTGCAKCTCTTGATC | 1662 |
| | ARA | |
| L2:95 | ACTAGCTTGTCGTAATAATGGCGGCATACTAKCAGTAGT | 1663 |
| | AGG | |
| L2:96 | CMAAGCTTGTCGTAATAATGGCGGCATACTAKCAGTAGT | 1664 |
| | AGG | |
| L2:97 | TACAGCTTGTCGTAATAATGGCGGCATACTAKCAGTAGT | 1665 |
| | AGG | |
| L2:98 | GAATAAGAAGGCTGGCTCTGCACCTTGGTGATCCAATAA | 1666 |
| | TTC | |
| L2:99 | GAATAAGAAGGCTGGCTCTGCACCTTGGTGATCTGMTAA | 1667 |
| | TTC | |
| L2:100 | GAATAAGAAGGCTGGCTCTGCACCTTGGTGATCCTTTAA | 1668 |
| | TTC | |
| L2:101 | TTTAAGTTGTTTTTCTAATCCGCATATGATCAATTCAAG | 1669 |
| | GCC | |
| L2:102 | GGGAACTTCGGCGCGCCTTAAGACCCACTTTCGSA | 1670 |

### Third Round Library Design and Screening

### Library L6: Shuffling for Enhanced Chlorsulfuron Response

Since clones L2-14 and L2-18 had the best chlorsulfuron activity profile from library L2, their amino acid diversity was used as the basis for the next round of shuffling. In addition to the diversity provided by these backbone sequences, additional residue changes thought to enhance packing of chlorsulfuron based on the 3D model predictions were included. New amino acid positions targeted were 67, 109, 112 and 173 (see, Table 12). Substitution of Gln (Q) at position 108 and Val (V) at position 170 were shown to likely be important changes in library L4 for gaining enhanced SU responsiveness and so were varied here as well. A summary of the diversity chose is shown in Table 12. The oligonucleotides designed and used to generate library 6 are shown in Table 13.

Library L6 was assembled, rescued, ligated into pVER7314, transformed into E. coli KM3 and plated out onto LB carbenicillin/kanamycin, and carbenicillin only control media as before. Library plates were then picked into 42 384-well microtiter plates (˜16,000 clones) containing 60 μl LB carbenicillin (Cb) broth per well. After overnight growth at 37° C. the cultures were stamped onto M9 assay plates containing no inducer, 0.2 ppm, and 2.0 ppm chlorsulfuron as test inducer. Following incubation at 30° C. for ˜48 hrs, putative hits responding to chlorsulfuron treatment as determined by increased blue colony color were re-arrayed into six 96-well microtiter plates and used to stamp a fresh set of M9 assay plates to confirm the above results. For a more detailed analysis of the relative induction by chlorsulfuron, digital photographs were taken of the plates after various time points of incubation at 30° C. and colony color intensity measured using the digital image analysis freeware program ImageJ (Rasband, US National Institutes of Health, Bethesda, Md., USA, rsb.info.nih.gov/ij/, 1997-2007). Using these results enabled ranking of clones in multiplex format by background activity (no inducer), activation with low or high level inducer application (blue color with inducer), and fold activation (activation divided by background). Activation studies using 0.2 μg/ml chlorsulfuron as inducer for the top set of clones shows an approximately 3 fold improvement in activation while obtaining lower un-induced levels of expression (Data not shown.) In addition to this analysis, DNA sequence information for most clones (490 clones) was obtained and the deduced polypeptides aligned with each other as well as with their corresponding activity information. From this analysis sequence-activity relationships were derived. (Data not shown.) Residues biased for improved activity are indicated in larger bold type. Briefly, C at position 100, and Q at positions 108 and 109 strongly correlated with activation, while R at position 138, L at position 170, and A or G at position 173 were highly preferred in clones with the lowest background activity. Though some positions were strongly biased, i.e., observed more frequently in the selected population, the entirety of introduced diversity was observed in the full hit population. This information will aid in the design of further libraries to improve responsiveness to chlorsulfuron.

TABLE 12

| Sequence | Amino acid residue position | | | | | | | | | | | | | | |
|---|---|---|---|---|---|---|---|---|---|---|---|---|---|---|---|
| Name | 60 | 64 | 67 | 82 | 86 | 100 | 105 | 108 | 109 | 112 | 113 | 116 | 134 | 138 | 139 |
| Library | | A | M | N | | C | | Q | M | S | | M | M | G | N |
| Diversity | | Q | Y | T | | W | | K | L | T | | Q | V | R | V |
| | | | F | | | | | | Q | A | | | | | |
| | | | L | | | | | | H | G | | | | | |
| | | | I | | | | | | | | | | | | |
| | | | V | | | | | | | | | | | | |
| wt reference | L | H | F | N | F | H | P | K | Q | T | L | Q | L | G | H |
| L2-14 | M | A | F | N | M | C | W | K | Q | T | A | M | V | R | V |
| L2-18 | M | Q | F | T | M | W | W | K | Q | T | A | Q | M | R | N |
| L6-1B03 | M | A | I | N | M | C | W | Q | Q | A | A | M | V | R | V |
| L6-2C09 | M | Q | Y | T | M | C | W | Q | L | T | A | Q | M | R | V |
| L6-2D07 | M | Q | F | T | M | C | W | Q | Q | T | A | M | M | R | V |
| L6-3H02 | M | A | Y | T | M | C | W | Q | H | S | A | M | V | R | V |
| L6-4D10 | M | Q | Y | N | M | C | W | K | Q | S | A | M | V | R | V |
| L6-5F05 | M | A | I | N | M | C | W | Q | Q | A | A | Q | V | R | V |
| L6-5G06 | M | Q | Y | N | M | C | W | Q | Q | T | A | Q | V | R | V |
| L6-5H06 | M | Q | I | N | M | C | W | K | Q | T | A | M | V | R | V |
| L6-5H12 | M | A | Y | N | M | C | W | K | Q | T | A | Q | M | R | V |
| L6-6F07 | M | A | L | T | M | C | W | Q | Q | S | A | M | M | R | V |
| Bias in top | | none | Y | N | | C | | Q | Q | none | | none | V | R | V |
| population | | | | | | | | | | | | | | | |

| Sequence | Amino acid residue position | | | | | | | | | | |
|---|---|---|---|---|---|---|---|---|---|---|---|
| Name | 147 | 151 | 164 | 170 | 173 | 174 | 177 | 178 | | | |
| Library | | S | | L | G | L | | | 0.2 ppm | Control | 0.2 ppm |
| Diversity | | L | | A | A | W | | | 48 hr | 84 hr | 48 hr/ |
| | | | | V | V | | | | | | Control |
| | | | | | | | | | | | 84 hr |
| wt reference | E | H | D | L | A | I | F | D | 5.2 | 5.3 | 1.0 |
| L2-14 | F | S | A | L | A | L | K | D | 11.8 | 6.6 | 1.8 |
| L2-18 | F | L | A | L | A | W | K | D | 5.9 | 5.7 | 1.0 |
| L6-1B03 | F | S | A | L | A | W | K | D | 30.0 | 6.6 | 4.6 |
| L6-2C09 | F | L | A | L | A | W | K | D | 13.6 | 5.2 | 2.6 |
| L6-2D07 | F | S | A | V | A | W | K | D | 20.0 | 5.8 | 3.4 |
| L6-3H02 | F | S | A | V | A | W | K | V | 15.8 | 5.6 | 2.8 |
| L6-4D10 | F | S | A | L | A | W | K | D | 18.4 | 5.0 | 3.7 |
| L6-5F05 | F | L | A | L | A | W | K | D | 22.0 | 5.4 | 4.1 |
| L6-5G06 | F | L | A | L | G | W | K | D | 34.4 | 7.0 | 4.9 |
| L6-5H06 | F | L | A | V | A | W | K | D | 13.7 | 5.1 | 2.7 |
| L6-5H12 | F | L | A | V | A | W | K | D | 23.7 | 5.7 | 4.2 |
| L6-6F07 | F | S | A | L | A | W | K | D | 11.6 | 5.1 | 2.3 |
| Bias in top | | none | | L | A/G | W | | D | | | |
| population | | | | | | | | | | | |

TABLE 13

| Oligo | Sequence | SEQ ID |
|---|---|---|
| L6:1 | TATTGGCATGTAAAAAATAAGCGAGCTCTGCTCGACG | 1671 |
| | CCTTA | |
| L6:2 | GCAGAGCCAGCCTTCTTATTCGGCCTTGAATTGATCA | 1672 |
| | TATGC | |
| L6:3 | ATATAATGCATTCTCTAGTGAAAAACCTTGTTGGCAT | 1673 |
| | AAAAA | |
| L6:4 | TTTAAGTTGTTTTTCTAATCCGCATATGATCAATTCA | 1674 |
| | AGGCC | |
| L6:5 | TTAGAAGGGGAAAGCTGGCAAGATTTTTTACGTAATA | 1675 |
| | MTGCT | |
| L6:6 | TAAAGCACATCTCATACTTTTAGCAKTATTACGTAAA | 1676 |
| | AAATC | |
| L6:7 | TTGCCAGCTTTCCCCTTCTAAAGGGCAMAHGTGAGTT | 1677 |
| | GCGTG | |
| L6:8 | TTGCCAGCTTTCCCCTTCTAAAGGGCAATAGTGAGTT | 1678 |
| | GCGTG | |
| L6:9 | GAATAAGAAGGCTGGCTCTGCACCTTGGTGATCCTTT | 1679 |
| | AATTC | |
| L6:10 | GCCATTGAGATGATGGATAGGCACGCAACTCACTATT | 1680 |
| | GCCCT | |
| L6:11 | RSTGCTGAAAATATGTTAGCCTTTTTATGCCAACAAG | 1681 |
| | GTTTT | |
| L6:12 | TTTACTTTAGGTTGCGTATTGTTTGATCAAGAGCTCC | 1682 |
| | AAGTC | |
| L6:13 | TGTTTCCCTTTCTTCTTTAGCGACTTGGAGCTCTTGA | 1683 |
| | TCAAA | |
| L6:14 | GCCATTGAGATGATGGATAGGCACGCAACTCACDTKT | 1684 |
| | GCCCT | |
| L6:15 | GCCATTGAGATGATGGATAGGCACCAAACTCACDTKT | 1685 |
| | GCCCT | |
| L6:16 | GCCATTGAGATGATGGATAGGCACCAAACTCACTATT | 1686 |
| | GCCCT | |
| L6:17 | AAAAGTATGAGATGTGCTTTACTAAGCCATCGCGATG | 1687 |
| | GAGCA | |
| L6:18 | AAAGTATGKTTAGGTACACGCTGGACAGAAMAACAWT | 1688 |
| | ATGAA | |
| L6:19 | AAAGTATGKTTAGGTACACGCTGGACAGAAMAAWTGT | 1689 |
| | ATGAA | |
| L6:20 | RSTGCTGAAAATCAATTAGCCTTTTTATGCCAACAAG | 1690 |
| | GTTTT | |
| L6:21 | TCACTAGAGAATGCATTATATGCARTGAGTGCGTGGR | 1691 |
| | GGGTG | |
| L6:22 | TCACTAGAGAATGCATTATATGCARTGAGTGCGTGGR | 1692 |
| | GGAAC | |
| L6:23 | TTTACTTTAGGTTGCGTATTGTTTGATCAAGAGAGCC | 1693 |
| | AAGTC | |
| L6:24 | GCTAAAGAAGAAAGGGAAACACCTACTACTGCTAGTA | 1694 |
| | TGCCG | |
| L6:25 | CCATTAKTGCGACAAGBTTKGGAATTAAAGGATCACC | 1695 |
| | AAGGT | |
| L6:26 | CCATTAGCCCGACAAGBTTKGGAATTAAAGGATCACC | 1696 |
| | AAGGT | |
| L6:27 | GGATTAGAAAAACAACTTAAATGCGAAAGTGGGTCTT | 1697 |
| | AA | |
| L6:28 | CCTATCCATCATCTCAATGGCTAAGGCGTCGAGCAGA | 1698 |
| | GCTCG | |
| L6:29 | TTGCCAGCTTTCCCCTTCTAAAGGGCAMAHGTGAGTT | 1699 |
| | TGGTG | |
| L6:30 | TTGCCAGCTTTCCCCTTCTAAAGGGCAATAGTGAGTT | 1700 |
| | TGGTG | |
| L6:31 | GCGTGTACCTAAMCATACTTTTGCTCCATCGCGATGG | 1701 |
| | CTTAG | |
| L6:32 | GGCTAACATATTTTCAGCASYTTCATAWTGTTKTTCT | 1702 |
| | GTCCA | |
| L6:33 | GGCTAATTGATTTTCAGCASYTTCATAWTGTTKTTCT | 1703 |
| | GTCCA | |
| L6:34 | GGCTAACATATTTTCAGCASYTTCATACAWTTKTTCT | 1704 |
| | GTCCA | |
| L6:35 | GGCTAATTGATTTTCAGCASYTTCATACAWTTKTTCT | 1705 |
| | GTCCA | |
| L6:36 | CAATACGCAACCTAAAGTAAACACCCYCACAGCACTC | 1706 |
| | AYTGC | |
| L6:37 | CAATACGCAACCTAAAGTAAAGTTCCYCACAGCACTC | 1707 |
| | AYTGC | |
| L6:38 | TGTTTCCCTTTCTTCTTTAGCGACTTGGCTCTCTTGA | 1708 |
| | TCAAA | |
| L6:39 | CMAAVCTTGTCGCAMTAATGGCGGCATACTAGCAGTA | 1709 |
| | GTAGG | |
| L6:40 | CMAAVCTTGTCGGGCTAATGGCGGCATACTAGCAGTA | 1710 |
| | GTAGG | |
| L6:41 | GGGAACTTCGGCGCGCCTTAAGACCCACTTTCGCA | 1711 |

### B. Fourth Round Shuffling:

Library L8 construction and screening. Fourth round shuffling incorporated the best diversity from Rd3 shuffling (BB1860) as well as computational diversity (Table 14). The fully synthetic library was constructed from oligonucleotides shown in Tables 15A and 15B. As diversity was very high the library oligo mix was spiked into the parental hit variant oligo mix (5, 10, and 25% mixes) to titer down the number of residue changes per clone. In addition, to varying residues for Cs activity, seven residues (C68, C86, C88, C121, C144, C195, and C203) were varied with TetR family phylogenetic substitutions in an attempt to reduce the number of cysteine residues in the repressor. The PCR assembled libraries were cloned Sac1/Asc1 into pVER7334. This plasmid encodes P_{BAD} promoter controlled expression of a plant optimized TetR DNA binding domain fused to the wt ligand binding domain of TetR(B) encoded by native Tn10 sequence on a Sac1 to Asc1 fragment. Approximately 15,000 clones were screened for blue colony color on the M9 Xgal assay plates +/−200 ppb Chlorsulfuron (Cs). Clones were ranked by ratio of color with inducer after 24 hrs incubation over colony color without inducer for 48 hrs of incubation. The sequence trend in the overall larger population of hits (first re-array) was that L55, R104, W105 and L170 were maintained while the C144A substitution was highly preferred. Sequence trends within the hit population were then noted with respect to repression, induction and fold induction (which corrects for leakiness). For repression C68L and C144A are favored in the highly repressed population: 57% and 93% in the top 40 repressed clones vs. 35% and 66% for the remaining 209 clones, respectively. the sequence analysis reveals that substitutions V134L and S135 to E, D, T, or Q were overrepresented. A sequence alignment of the top 20 clones is shown in Table 16.

TABLE 14
Library diversity summary for fourth, fifth and
sixth round Chlorsulfuron repressor shuffling.

| Sequence | TetR(B) | | | |
|---|---|---|---|---|
| position | Sequence | L8 | CsL3 | CsL4.2 |
| 55 | L | [symbol] | — | — |
| 60 | L | ML | HMN | [symbol] |
| 64 | H | QILV | [symbol] | [symbol] |
| 67 | F | Y | | — |
| 68 | C | LSC | L | L |
| 78 | F | — | — | [symbol] |
| 82 | N | NLIV | [symbol] | FY |
| 86 | F | WFYILMC | [symbol] | M |
| 88 | C | RNC | R | CLR |
| 100 | H | WMVC | AS | AS |
| 104 | R | [symbol] | R | R |
| 105 | P | [symbol] | W | W |
| 108 | K | Q | — | — |
| 112 | T | ST | — | — |
| 113 | L | AV | [symbol] | A |
| 116 | Q | M | [symbol] | M |
| 117 | L | ML | — | — |
| 121 | C | TC | T | T |
| 131 | L | ML | — | — |
| 134 | L | [symbol] | [symbol] | T |
| 135 | S | [symbol] | [symbol] | DS |
| 137 | V | AV | — | — |
| 138 | G | R | [symbol] | R |
| 139 | H | IV | [symbol] | V |
| 144 | C | [symbol] | A | A |
| 147 | E | LGKCRFWV | [symbol] | [symbol] |
| 151 | H | S | GQS | [symbol] |
| 155 | K | — | — | KN |
| 163 | T | — | — | PT |
| 165 | S | — | — | RS |
| 170 | L | [symbol] | L | — |
| 173 | A | — | — | — |
| 174 | I | W | W | W |
| 177 | F | QK | K | K |
| 178 | D | — | — | [symbol] |
| 195 | C | SRAC | A | A |
| 203 | C | SRAC | R | R |

TABLE 15A
Library L8 assembly oligonucleotides

| | SEQ ID | | |
|---|---|---|---|
| Oligo | NO | Sequence | Group |
| L8:1 | 1712 | CACACAGGAATCCATGGCCAGACTCGACAAGAGCAAGGTG | 1 |
| L8:2 | 1713 | ATCAACAGCGCACTGGAGCTGCTGAACGAGGTCGGAATCGAA | 2 |
| L8:3 | 1714 | GGCCTCACAACCCGTAAACTCGCCCAGAAGCTCGGGGTAGAG | 3 |
| L8:4 | 1715 | CAGCCTACATTGTATTGGCACGTCAAGAACAAGCGAGCTCTG | 4 |
| L8:5 | 1716 | CTAGACGCCWTGGCCATTGAGATGWTGGATAGGCACCAWACC | 5 |
| L8:6 | 1717 | CTAGACGCCWTGGCCATTGAGATGWTGGATAGGCACVTTACC | |
| L8:7 | 1718 | CACTACTGCCCTTTGGAAGGGGAAAGCTGGCAAGACTTCTTG | 6 |
| L8:8 | 1719 | AGGAACAACGCTAAGAGCWTSAGATGTGCTTTGCTCAGTCAC | 7 |
| L8:9 | 1720 | AGGAACAACGCTAAGAGCTGGAGATGTGCTTTGCTCAGTCAC | |
| L8:10 | 1721 | AGGAACAACGCTAAGAGCTACAGATGTGCTTTGCTCAGTCAC | |
| L8:11 | 1722 | AGGAACVTTGCTAAGAGCWTSAGATGTGCTTTGCTCAGTCAC | |
| L8:12 | 1723 | AGGAACVTTGCTAAGAGCTGGAGATGTGCTTTGCTCAGTCAC | |
| L8:13 | 1724 | AGGAACVTTGCTAAGAGCTACAGATGTGCTTTGCTCAGTCAC | |
| L8:14 | 1725 | CGTGATGGAGCCAAGGTCTGSCTAGGTACAGCGTKGACGGAG | 8 |
| L8:15 | 1726 | CGTGATGGAGCCAAGGTCTGSCTAGGTACAGCGTWCACGGAG | |
| L8:16 | 1727 | CGTGATGGAGCCAAGGTCTGSCTAGGTACASGGTKGACGGAG | |
| L8:17 | 1728 | CGTGATGGAGCCAAGGTCTGSCTAGGTACASGGTWCACGGAG | |
| L8:18 | 1729 | CGTGATGGAGCCAAGGTCRTGCTAGGTACAGCGTKGACGGAG | |
| L8:19 | 1730 | CGTGATGGAGCCAAGGTCRTGCTAGGTACAGCGTWCACGGAG | |
| L8:20 | 1731 | CGTGATGGAGCCAAGGTCRTGCTAGGTACASGGTKGACGGAG | |
| L8:21 | 1732 | CGTGATGGAGCCAAGGTCRTGCTAGGTACASGGTWCACGGAG | |
| L8:22 | 1733 | CAACAGTATGAAWCTGYGGAGAACATGWTGGCCTTCCTGTGC | 9 |
| L8:23 | 1734 | CAACAAGGTTTCTCCCTTGAGAATGCCWTGTACGCAVTCDCG | 10 |
| L8:24 | 1735 | CAACAAGGTTTCTCCCTTGAGAATGCCWTGTACGCAVTCMAG | |
| L8:25 | 1736 | CAACAAGGTTTCTCCCTTGAGAATGCCWTGTACGCAVTCYGC | |
| L8:26 | 1737 | CAACAAGGTTTCTCCCTTGAGAATGCCWTGTACGCAVTCGAM | |
| L8:27 | 1738 | GCTGYGCGGRTTTTCACTCTGGGTTGCGTATTGBKGGATCAA | 11 |
| L8:28 | 1739 | GCTGYGCGGRTTTTCACTCTGGGTTGCGTATTGAAGGATCAA | |
| L8:29 | 1740 | GCTGYGCGGRTTTTCACTCTGGGTTGCGTATTGTKTGATCAA | |
| L8:30 | 1741 | GAGTCCCAAGTCGCTAAGGAGGAGAGGGAAACACCTACTACT | 12 |
| L8:31 | 1742 | GATAGTATGCCGCCACTGMTTCGACAAGCTTGGGAACTCMAA | 13 |
| L8:32 | 1743 | GATCACCAAGGTGCAGAGCCAGCCTTCCTGTTCGGCCTTGAA | 14 |
| L8:33 | 1744 | TTGATCATATGCGGATTGGAGAAGCAGCTGAAGTGTGAAAGT | 15 |
| L8:34 | 1745 | GGGTCTTAAGGCGCGCCGAAGTTCCC | 16 |
| L8:35 | 1746 | CAGCTCCAGTGCGCTGTTGATCACCTTGCTCTTGTCGAGTCT | 17 |
| L8:36 | 1747 | GAGTTTACGGGTTGTGAGGCCTTCGATTCCGACCTCGTTCAG | 18 |
| L8:37 | 1748 | GTGCCAATACAATGTAGGCTGCTCTACCCCGAGCTTCTGGGC | 19 |
| L8:38 | 1749 | CTCAATGGCCAWGGCGTCTAGCAGAGCTCGCTTGTTCTTGAC | 20 |
| L8:39 | 1750 | CCCTTCCAAAGGGCAGTAGTGGGTWTGGTGCCTATCCAWCAT | 21 |
| L8:40 | 1751 | CCCTTCCAAAGGGCAGTAGTGGGTAABGTGCCTATCCAWCAT | |
| L8:41 | 1752 | SAWGCTCTTAGCGTTGTTCCTCAAGAAGTCTTGCCAGCTTTC | 22 |
| L8:42 | 1753 | CCAGCTCTTAGCGTTGTTCCTCAAGAAGTCTTGCCAGCTTTC | |
| L8:43 | 1754 | GTAGCTCTTAGCGTTGTTCCTCAAGAAGTCTTGCCAGCTTTC | |
| L8:44 | 1755 | SAWGCTCTTAGCAABGTTCCTCAAGAAGTCTTGCCAGCTTTC | |
| L8:45 | 1756 | CCAGCTCTTAGCAABGTTCCTCAAGAAGTCTTGCCAGCTTTC | |
| L8:46 | 1757 | GTAGCTCTTAGCAABGTTCCTCAAGAAGTCTTGCCAGCTTTC | |
| L8:47 | 1758 | SCAGACCTTGGCTCCATCACGGTGACTGAGCAAAGCACATCT | 23 |
| L8:48 | 1759 | CAYGACCTTGGCTCCATCACGGTGACTGAGCAAAGCACATCT | |
| L8:49 | 1760 | CTCCRCAGWTTCATACTGTTGCTCCGTCMACGCTGTACCTAG | 24 |
| L8:50 | 1761 | CTCCRCAGWTTCATACTGTTGCTCCGTGWACGCTGTACCTAG | |
| L8:51 | 1762 | CTCCRCAGWTTCATACTGTTGCTCCGTCMACCSTGTACCTAG | |
| L8:52 | 1763 | CTCCRCAGWTTCATACTGTTGCTCCGTGWACCSTGTACCTAG | |
| L8:53 | 1764 | CTCAAGGGAGAAACCTTGTTGGCACAGGAAGGCCAWCATGTT | 25 |
| L8:54 | 1765 | CAGAGTGAAAAYCCGCRCAGCCGHGABTGCGTACAWGGCATT | 26 |
| L8:55 | 1766 | CAGAGTGAAAAYCCGCRCAGCCTKGABTGCGTACAWGGCATT | |
| L8:56 | 1767 | CAGAGTGAAAAYCCGCRCAGCGCRGABTGCGTACAWGGCATT | |
| L8:57 | 1768 | CAGAGTGAAAAYCCGCRCAGCKTCGABTGCGTACAWGGCATT | |
| L8:58 | 1769 | CTCCTTAGCGACTTGGGACTCTTGATCCMVCAATACGCAACC | 27 |
| L8:59 | 1770 | CTCCTTAGCGACTTGGGACTCTTGATCCTTCAATACGCAACC | |
| L8:60 | 1771 | CTCCTTAGCGACTTGGGACTCTTGATCAMACAATACGCAACC | |
| L8:61 | 1772 | AAKCAGTGGCGGCATACTATCAGTAGTAGGTGTTTCCCTCTC | 28 |
| L8:62 | 1773 | TGGCTCTGCACCTTGGTGATCTTKGAGTTCCCAAGCTTGTCG | 29 |
| L8:63 | 1774 | CTCCAATCCGCATATGATCAATTCAAGGCCGAACAGGAAGGC | 30 |
| L8:64 | 1775 | CTTCGGCGCGCCTTAAGACCCACTTTCACACTTCAGCTGCTT | 31 |

TABLE 15B
Oligonucleotide mixes encoding parent clone for library L8.

| | SEQ | | |
|---|---|---|---|
| Oligo | ID NO | Oligo Sequence | Group |
| L6-4D10:01 | 1776 | CAGCCTACATTGTATTGGCACGTCAAGAACAAGCGAGCTCTG | 4 |
| L6-4D10:02 | 1777 | CTAGACGCCTTGGCCATTGAGATGATGGATAGGCACCAAACC | 5 |
| L6-4D10:03 | 1778 | CACTACTYGCCTTTGGAAGGGGAAAGCTGGCAAGACTTCTTG | 6 |
| L6-4D10:04 | 1779 | AGGAACAACGCTAAGAGCTGCAGACGTGCTTTGCTCAGTCAC | 7 |
| L6-4D10:05 | 1780 | AGGAACAACGCTAAGAGCTGCAGAAATGCTTTGCTCAGTCAC | |
| L6-4D10:06 | 1781 | CGTGATGGAGCCAAGGTCTGCCTAGGTACACGGTGGACGGAG | 8 |
| L6-4D10:07 | 1782 | CAACAGTATGAATCTGCGGAGAACATGTTGGCCTTCCTGACC | 9 |
| L6-4D10:08 | 1783 | CAACAAGGTTTCTCCCTTGAGAATGCCTTGTACGCAGTCTCC | 10 |
| L6-4D10:09 | 1784 | GCTGTGCGGGTTTTCACTCTGGGTTGGGTATTGTTCGATCAA | 11 |
| L6-4D10:10 | 1785 | GCTGTGCGGGTTTTCACTCTGGGTGCCGTATTGTTCGATCAA | |
| L6-4D10:11 | 1786 | GAGTCCCAAGTCGCTAAGGAGGAGAGGGAAACACCTACTACT | 12 |
| L6-4D10:12 | 1787 | GATAGTATGCCGCCACTGCTTCGACAAGCTTGGGAACTCAAA | 13 |
| L6-4D10:13 | 1788 | GATCACCAAGGTGCAGAGCCAGCCTTCCTGTTCGGCCTTGAA | 14 |
| L6-4D10:14 | 1789 | TTGATCATAKCCGGATTGGAGAAGCAGCTGAAGKCAGAAAGT | 15 |
| L6-4D10:15 | 1790 | TTGATCATAKCCGGATTGGAGAAGCAGCTGAAGAGAGAAAGT | |
| L6-4D10:16 | 1791 | TTGATCATACGCGGATTGGAGAAGCAGCTGAAGKCAGAAAGT | |
| L6-4D10:17 | 1792 | TTGATCATACGCGGATTGGAGAAGCAGCTGAAGAGAGAAAGT | |
| L6-4D10:18 | 1793 | GGGTCTTAATGATAGCTGCAGAAGGTACCTTGGCGCGCC | 16 |
| L6-4D10:19 | 1794 | CTCAATGGCCAAGGCGTCTAGCAGAGCTCGCTTGTTCTTGAC | 20 |
| L6-4D10:20 | 1795 | CCCTTCCAAAGGCRAGTAGTGGGTTTGGTGCCTATCCATCAT | 21 |
| L6-4D10:21 | 1796 | GCAGCTCTTAGCGTTGTTCCTCAAGAAGTCTTGCCAGCTTTC | 22 |
| L6-4D10:22 | 1797 | GCAGACCTTGGCTCCATCACGGTGACTGAGCAAAGCACGTCT | 23 |
| L6-4D10:23 | 1798 | GCAGACCTTGGCTCCATCACGGTGACTGAGCAAAGCATTTCT | |
| L6-4D10:24 | 1799 | CTCCGCAGATTCATACTGTTGCTCCGTCCACCGTGTACCTAG | 24 |
| L6-4D10:25 | 1800 | CTCAAGGGAGAAACCTTGTTGGGTCAGGAAGGCCAACATGTT | 25 |
| L6-4D10:26 | 1801 | CAGAGTGAAAACCCGCACAGCGGAGACTGCGTACAAGGCATT | 26 |
| L6-4D10:27 | 1802 | CTCCTTAGCGACTTGGGACTCTTGATCGAACAATACCCAACC | 27 |
| L6-4D10:28 | 1803 | CTCCTTAGCGACTTGGGACTCTTGATCGAACAATACGGCACC | |
| L6-4D10:29 | 1804 | AAGCAGTGGCGGCATACTATCAGTAGTAGGTGTTTCCCTCTC | 28 |
| L6-4D10:30 | 1805 | TGGCTCTGCACCTTGGTGATCTTTGAGTTCCCAAGCTTGTCG | 29 |
| L6-4D10:31 | 1806 | CTCCAATCCGGMTATGATCAATTCAAGGCCGAACAGGAAGGC | 30 |
| L6-4D10:32 | 1807 | CTCCAATCCGCGTATGATCAATTCAAGGCCGAACAGGAAGGC | |
| L6-4D10:33 | 1808 | CTGCAGCTATCATTAAGACCCACTTTCTGMCTTCAGCTGCTT | 31 |
| L6-4D10:34 | 1809 | CTGCAGCTATCATTAAGACCCACTTTCTCTCTTCAGCTGCTT | |

TABLE 16
Sequence alignment and relative performance of the top
20 L8 hits relative to parent clone L6-4D10.

| | Colony Assay | | | Residue and | | | | | | | | | | | | | |
|---|---|---|---|---|---|---|---|---|---|---|---|---|---|---|---|---|---|
| | Results | | | Sequence Position | | | | | | | | | | | | | |
| Clone | IND | REP | F. IND | 60 | 64 | 67 | 68 | 86 | 88 | 90 | 100 | 105 | 108 | 112 | 113 | 116 | 121 |
| TetR | ND | ND | ND | L | H | F | — | F | — | — | H | P | — | T | L | Q | — |
| L6-4D10 | 0.2 | 0.6 | 0.4 | M | Q | Y | C | C | C | L | C | W | K | S | A | M | C |
| L8-3F09 | 5.6 | 0.6 | 9.7 | — | — | — | S | L | — | — | — | — | Q | — | — | — | T |
| L8-1A04 | 12.2 | 2.0 | 6.2 | — | — | — | L | C | N | — | — | — | Q | T | — | — | T |
| L8-3B08 | 13.0 | 2.1 | 6.1 | — | — | — | S | L | — | — | — | — | Q | — | — | — | T |
| L8-1B12 | 12.5 | 2.4 | 5.1 | — | — | — | S | W | — | — | — | — | Q | — | — | — | T |
| L8-3D03 | 5.9 | 1.2 | 4.9 | — | — | — | L | C | N | — | — | — | Q | — | — | — | T |
| L8-2F12 | 2.7 | 0.7 | 3.6 | — | — | — | S | C | N | — | — | — | Q | — | — | — | T |
| L8-3F02 | 3.4 | 1.0 | 3.5 | — | — | — | S | C | R | — | — | — | Q | — | — | — | — |
| L8-3E05 | 1.4 | 0.4 | 3.4 | — | — | — | — | L | — | — | — | — | Q | — | — | — | T |
| L8-3A05 | 0.3 | 0.1 | 3.3 | — | — | — | S | C | N | — | — | — | Q | — | — | — | T |
| L8-3A04 | 0.5 | 0.1 | 3.3 | — | — | — | S | C | R | — | — | — | Q | — | — | — | T |
| L8-1A03 | 8.6 | 2.8 | 3.1 | — | — | — | S | C | N | — | — | — | Q | — | — | — | T |
| L8-3F01 | 1.7 | 0.6 | 3.0 | — | — | — | L | C | R | — | — | — | Q | T | — | — | T |
| L8-3A07 | 0.7 | 0.2 | 2.9 | — | — | — | S | M | — | — | — | — | Q | — | — | — | T |
| L8-1A06 | 2.1 | 0.8 | 2.7 | — | — | — | S | C | R | V | — | — | Q | — | — | — | T |
| L8-2H01 | 12.9 | 4.8 | 2.7 | — | — | — | S | C | R | — | — | — | Q | — | — | — | T |
| L8-3F08 | 1.5 | 0.6 | 2.7 | — | — | — | S | C | N | — | V | — | Q | — | — | — | T |
| L8-3A06 | 0.3 | 0.1 | 2.6 | — | — | — | L | C | N | — | — | — | Q | — | — | — | T |
| L8-1E04 | 1.5 | 0.6 | 2.5 | — | — | — | L | C | N | — | — | — | Q | — | — | — | T |
| L8-1A05 | 10.8 | 4.4 | 2.5 | — | — | — | S | C | R | — | — | — | Q | — | — | — | T |
| L8-3B03 | 0.6 | 0.3 | 2.4 | — | — | — | L | C | R | — | — | — | Q | — | — | — | T |

| | Residue and | | | | | | | | | | | | | | |
|---|---|---|---|---|---|---|---|---|---|---|---|---|---|---|---|
| | Sequence Position | | | | | | | | | | | | | | |
| Clone | 131 | 134 | 135 | 137 | 138 | 139 | 144 | 147 | 151 | 164 | 174 | 177 | 195 | 203 | 205 |
| TetR | — | L | — | — | G | H | — | E | H | D | I | F | — | — | — |
| L6-4D10 | L | V | S | V | R | V | C | F | S | A | W | K | C | C | S |
| L8-3F09 | — | L | T | — | — | — | W | — | — | D | — | — | A | R | — |
| L8-1A04 | — | L | E | A | — | — | A | — | — | D | — | — | S | R | — |
| L8-3B08 | — | — | D | A | — | — | W | — | — | D | — | — | R | R | — |
| L8-1B12 | — | L | E | — | — | — | W | — | — | D | — | — | A | A | — |
| L8-3D03 | — | L | D | — | — | — | A | — | — | D | — | — | R | S | — |
| L8-2F12 | — | L | Q | — | — | — | W | — | — | D | — | — | R | S | — |
| L8-3F02 | — | — | — | — | — | I | A | — | — | D | — | — | A | R | — |
| L8-3E05 | — | L | Q | — | — | I | — | — | — | D | — | — | R | R | — |
| L8-3A05 | — | — | — | — | — | — | A | — | — | D | — | — | S | S | — |
| L8-3A04 | — | — | — | — | — | — | A | — | — | D | — | — | R | R | C |
| L8-1A03 | — | L | D | — | — | — | A | — | — | D | — | — | A | R | — |
| L8-3F01 | — | — | — | — | — | — | A | — | — | D | — | — | A | R | — |
| L8-3A07 | — | — | — | — | — | — | W | — | — | D | — | — | A | R | — |
| L8-1A06 | — | — | — | — | — | — | A | — | — | D | — | — | A | R | — |
| L8-2H01 | — | L | E | — | — | — | A | — | — | D | — | — | R | R | — |
| L8-3F08 | M | L | E | A | — | I | W | — | — | D | — | — | R | S | — |
| L8-3A06 | — | — | — | — | — | — | A | — | — | D | — | — | S | R | — |
| L8-1E04 | — | — | — | — | — | — | A | Y | — | D | — | — | R | A | — |
| L8-1A05 | — | — | D | — | — | — | A | — | — | D | — | — | A | A | — |
| L8-3B03 | — | — | — | — | — | — | A | — | — | D | — | — | R | S | — |

Clones ranked by blue colony color intensity thru ImageJ analysis.
IND = induction with 200 ppb Cs at 24 hrs
REP = repression measured without inducer after 48 hrs
F. IND = fold induction: induction with 200 ppb Cs at 24 hrs/repression at 48 hrs

### C. Fifth Round Chlorsulfuron Repressor Shuffling

Saturation Mutagenesis of Ligand Binding Pocket:

To generate novel diversity for further rounds of shuffling residues 60, 64, 82, 86, 100, 104, 105, 113, 116, 134, 135, 138, 139, 147, 151, 174, and 177 in L8 hit L8-3F01 were subjected to NNK substitution mutagenesis with the following primers shown in Table 17.

TABLE 17
Oligonucleotides used for saturation mutagenesis of
putative ligand binding pocket residues.

| | Residue/ | | SEQ ID |
|---|---|---|---|
| Oligo | Strand | Sequence | NO |
| 3F1-60T | 60 top | CCTTGGCCATTGAGATGNNKGATAGGCACCAAACCCACTAC | 1810 |
| 3F1-60B | 60 bottom | GTAGTGGGTTTGGTGCCTATCMNNCATCTCAATGGCCAAGG | 1811 |
| 3F1-64T | 64 top | GAGATGATGGATAGGCACNNKACCCACTACTTGCCTTTG | 1812 |
| 3F1-64B | 64 bottom | CAAAGGCAAGTAGTGGGTMNNGTGCCTATCCATCATCTC | 1813 |
| 3F1-82T | 60 top | CAAGACTTCTTGAGGAACNNKGCTAAGAGCTGCAGACGTG | 1814 |
| 3F1-82B | 82 bottom | CACGTCTGCAGCTCTTAGCMNNGTTCCTCAAGAAGTCTTG | 1815 |
| 3F1-86T | 86 top | GAGGAACAACGCTAAGAGCNNKAGACGTGCTTTGCTCAGTC | 1816 |
| 3F1-86B | 86 bottom | GACTGAGCAAAGCACGTCTMNNGCTCTTAGCGTTGTTCCTC | 1817 |
| 3F1-100T | 100 top | CGTGATGGAGCCAAGGTCNNKCTAGGTACACGGTGGACG | 1818 |
| 3F1-100B | 100 bottom | CGTCCACCGTGTACCTAGMNNGACCTTGGCTCCATCACG | 1819 |
| 3F1-104T | 104 top | CAAGGTCTGCCTAGGTACANNKTGGACGGAGCAACAGTATG | 1820 |
| 3F1-104B | 104 bottom | CATACTGTTGCTCCGTCCAMNNTGTACCTAGGCAGACCTTG | 1821 |
| 3F1-105T | 105 top | GTCTGCCTAGGTACACGGNNKACGGAGCAACAGTATGAAAC | 1822 |
| 3F1-105B | 105 bottom | GTTTCATACTGTTGCTCCGTMNNCCGTGTACCTAGGCAGAC | 1823 |
| 3F1-113T | 113 top primer | GAGCAACAGTATGAAACTNNKGAGAACATGTTGGCCTTCC | 1824 |
| 3F1-113B | 113 bottom | GGAAGGCCAACATGTTCTCMNNAGTTTCATACTGTTGCTC | 1825 |
| 3F1-116T | 116 top | GTATGAAACTGCGGAGAACNNKTTGGCCTTCCTGACCCAAC | 1826 |
| 3F1-116B | 116 bottom | GTTGGGTCAGGAAGGCCAAMNNGTTCTCCGCAGTTTCATAC | 1827 |
| 3F1-134T | 134 top | GAGAATGCCTTGTACGCANNKTCCGCTGTGCGGGTTTTCAC | 1828 |
| 3F1-134B | 134 bottom | GTGAAAACCCGCACAGCGGAMNNTGCGTACAAGGCATTCTC | 1829 |
| 3F1-135T | 135 top | GAATGCCTTGTACGCAGTCNNKGCTGTGCGGGTTTTCACTC | 1830 |
| 3F1-135B | 135 bottom | GAGTGAAAACCCGCACAGCMNNGACTGCGTACAAGGCATTC | 1831 |
| 3F1-138T | 138 top | GTACGCAGTCTCCGCTGTGNNKGTTTTCACTCTGGGTGCC | 1832 |
| 3F1-138B | 138 bottom | GGCACCCAGAGTGAAAACMNNCACAGCGGAGACTGCGTAC | 1833 |
| 3F1-139T | 139 top | ACGCAGTCTCCGCTGTGCGGNNKTTCACTCTGGGTGCCGTA | 1834 |
| 3F1-139B | 139 bottom | TACGGCACCCAGAGTGAAMNNCCGCACAGCGGAGACTGCGT | 1835 |
| 3F1-147T | 147 top | CACTCTGGGTGCCGTATTGNNKGATCAAGAGTCCCAAGTC | 1836 |
| 3F1-147B | 147 bottom | GACTTGGGACTCTTGATCMNNCAATACGGCACCCAGAGTG | 1837 |
| 3F1-151T | 151 top | CGTATTGTTCGATCAAGAGNNKCAAGTCGCTAAGGAGGAGAG | 1838 |
| 3F1-151B | 151B | CTCTCCTCCTTAGCGACTTGMNNCTCTTGATCGAACAATACG | 1839 |
| 3F1-174T | 174 top | GCCACTGCTTCGACAAGCTNNKGAACTCAAAGATCACCAAG | 1840 |
| 3F1-174B | 174 bottom | CTTGGTGATCTTTGAGTTCMNNAGCTTGTCGAAGCAGTGGC | 1841 |
| 3F1-177T | 177 top | TCGACAAGCTTGGGAACTCNNKGATCACCAAGGTGCAGAGC | 1842 |
| 3F1-177B | 177 bottom | GCTCTGCACCTTGGTGATCMNNGAGTTCCCAAGCTTGTCGA | 1843 |

Mutagenesis reactions were transformed into library strain Km3 and 96 colonies tested for substitution by DNA sequence analysis. Substitutions representing each possible residue at each position were then re-arrayed in triplicate onto M9 X-gal assay plates with 0, 20 and 200 ppb Chlorsulfuron. Plates were incubated at 37° C. for 24 and 48 hrs prior to imaging. Residue substitutions were then ranked by activation (emphasis on 20 ppb Cs) and repression characteristics (emphasis on 48 hr time point). The mutation with the greatest impact on activity was substitution of residue N82 to phenylalanine or tyrosine. Tryptophan substitution also improved activity at N82 but not nearly as much as either phe or tyr. Substitutions S135D, S135E, F147Q, F147V and S151Q all dramatically increase sensitivity to Chlorsulfuron induction however partially at the expense of repressor function. All other preferred substitutions shown in Table 18 either improved repression or improved sensitivity to inducer without compromising repressor function. Certain residues were indispensible to function such as R104, W105, and W174 as substitutions were not allowed. Other residue positions such as R138 and K177 were also flagged as critical since functional substitutions were extremely limited.

TABLE 18
Summary of saturation mutagenesis results.

| | Residue targeted for mutagenesis | | | | | | | | | | | | | | | | |
|---|---|---|---|---|---|---|---|---|---|---|---|---|---|---|---|---|---|
| | M60 | Q64 | N82 | C86 | C100 | R104 | W105 | A113 | M116 | V134 | S135 | R138 | V139 | F147 | S151 | W174 | K177 |
| Top | H | D | [symbol] | G | A | [symbol] | [symbol] | A | L | L | D | H | I | F | G | [symbol] | [symbol] |
| Substitutions | M | E | [symbol] | M | C | | | G | M | T | E | [symbol] | L | L | Q | | R |
| | N | G | | ST | S | | | V | Q | V | G | | V | M | S | | |
| | | Q | | | | | | | | | S | | | Q | | | |
| | | S | | | | | | | | | | | | V | | | |

Bold = highly sensitive response but slightly leaky;
Bold and italic = highly selected residues;
*= only residue that functions at the respective position

Library CsL3 construction and screening: Based on the IVM results the top performing residue substitutions were incorporated into library CsL3 (Table 14). The library was assembled with the oligonucleotides shown below in Table 19. The first and last primers in each set were used as rescue primers. To enable purification of hit proteins, a 6×His-tag between was added to the C-terminus of the ligand binding domain of each clone during the assembly and rescue process. The library was then inserted into pVER7334 Sac1/Asc1, transformed into E. coli assay strain Km3 and selected on LB+40 ug/ml Kanamycin and 50 ug/ml Carbenicillin. Approximately 10,000 colonies were then re-arrayed into 384-well format, and replica plated onto M9 Xgal assay medium containing 0 or 20 ppb Cs. Colony color was then assessed at 24 and 96 hrs of incubation at 37° C. Results showed that residue substitutions N82F, V134T, and F147Q were highly preferred as was the maintenance of residues Q64, A113, M116, 5135, R138, and V139. Interestingly the very best hits had a random F147L substitution resulting in an additional ˜2× increase in activity over the next best clones. Also, while the C86M substitution was less frequent in the overall hit population it occurred in all top 26 clones.

TABLE 19
Oligonucleotides encoding library CsL3.

| | SEQ ID | | |
|---|---|---|---|
| Oligo | NO | Sequence | Group |
| CsL3:1 | 1844 | TGGCACGTCAAGAACAAGCGAGCTCTGCTAGACGCCTTGGCC | 1 |
| CsL3:2 | 1845 | ATTGAGATGMATGATAGGCACRGCACCCACTACTTGCCTTTG | 2 |
| CsL3:3 | 1846 | ATTGAGATGMATGATAGGCACCAGACCCACTACTTGCCTTTG | |
| CsL3:4 | 1847 | ATTGAGATGATGGATAGGCACRGCACCCACTACTTGCCTTTG | |
| CsL3:5 | 1848 | ATTGAGATGATGGATAGGCACCAGACCCACTACTTGCCTTTG | |
| CsL3:6 | 1849 | GAAGGGGAAAGCTGGCAAGACTTCTTGAGGAACTWCGCTAAG | 3 |
| CsL3:7 | 1850 | AGCTCCCGACGTGCTTTGCTCAGTCACCGTGATGGAGCCAAG | 4 |
| CsL3:8 | 1851 | AGCATGCGACGTGCTTTGCTCAGTCACCGTGATGGAGCCAAG | |
| CsL3:9 | 1852 | GTCKCGCTTGGTACACGGTGGACGGAGCAACAGTATGAAACT | 5 |
| CsL3:10 | 1853 | GSAGAGAACWTGTTGGCCTTCCTGACCCAACAAGGTTTCTCC | 6 |
| CsL3:11 | 1854 | CTTGAGAATGCCTTGTACGCAACCGRCGCTGTGCRTRTTTTC | 7 |
| CsL3:12 | 1855 | CTTGAGAATGCCTTGTACGCAACCTCAGCTGTGCRTRTTTTC | |
| CsL3:13 | 1856 | CTTGAGAATGCCTTGTACGCASTGGRCGCTGTGCRTRTTTTC | |
| CsL3:14 | 1857 | CTTGAGAATGCCTTGTACGCASTGTCAGCTGTGCRTRTTTTC | |
| CsL3:15 | 1858 | ACTCTGGGTGCCGTATTGGTGGATCAAGAGRGCCAAGTCGCT | 8 |
| CsL3:16 | 1859 | ACTCTGGGTGCCGTATTGGTGGATCAAGAGCAGCAAGTCGCT | |
| CsL3:17 | 1860 | ACTCTGGGTGCCGTATTGCAAGATCAAGAGRGCCAAGTCGCT | |
| CsL3:18 | 1861 | ACTCTGGGTGCCGTATTGCAAGATCAAGAGCAGCAAGTCGCT | |
| CsL3:19 | 1862 | AAGGAGGAGAGGGAAACACCTACTACTGATAGTATGCCGCCA | 9 |
| CsL3:20 | 1863 | CTGCTTCGACAAGCCTGGGAACTCAAAGATCACCAAGGTGCA | 10 |
| CsL3:21 | 1864 | GAGCCAGCCTTCCTGTTCGGCCTTGAATTGATCATAGCCGGA | 11 |
| CsL3:22 | 1865 | TTGGAGAAGCAGCTGAAGAGAGAAAGTGGGTCTCACCATCAC | 12 |
| CsL3:23 | 1866 | GTGCCTATCATKCATCTCAATGGCCAAGGCGTCTAGCAGAGC | 13 |
| CsL3:24 | 1867 | GTGCCTATCCATCATCTCAATGGCCAAGGCGTCTAGCAGAGC | |
| CsL3:25 | 1868 | GTCTTGCCAGCTTTCCCCTTCCAAAGGCAAGTAGTGGGTGCT | 14 |
| CsL3:26 | 1869 | GTCTTGCCAGCTTTCCCCTTCCAAAGGCAAGTAGTGGGTGCC | |
| CsL3:27 | 1870 | GTCTTGCCAGCTTTCCCCTTCCAAAGGCAAGTAGTGGGTCTG | |
| CsL3:28 | 1871 | GAGCAAAGCACGTCGGGAGCTCTTAGCGWAGTTCCTCAAGAA | 15 |
| CsL3:29 | 1872 | GAGCAAAGCACGTCGCATGCTCTTAGCGWAGTTCCTCAAGAA | |
| CsL3:30 | 1873 | CCACCGTGTACCAAGCGMGACCTTGGCTCCATCACGGTGACT | 16 |
| CsL3:31 | 1874 | GAAGGCCAACAWGTTCTCTSCAGTTTCATACTGTTGCTCCGT | 17 |
| CsL3:32 | 1875 | TGCGTACAAGGCATTCTCAAGGGAGAAACCTTGTTGGGTCAG | 18 |
| CsL3:33 | 1876 | CACCAATACGGCACCCAGAGTGAAAAYAYGCACAGCGYCGGT | 19 |
| CsL3:34 | 1877 | TTGCAATACGGCACCCAGAGTGAAAAYAYGCACAGCGYCGGT | |
| CsL3:35 | 1878 | CACCAATACGGCACCCAGAGTGAAAAYAYGCACAGCTGAGGT | |
| CsL3:36 | 1879 | TTGCAATACGGCACCCAGAGTGAAAAYAYGCACAGCTGAGGT | |
| CsL3:37 | 1880 | CACCAATACGGCACCCAGAGTGAAAAYAYGCACAGCGYCCAC | |
| CsL3:38 | 1881 | CACCAATACGGCACCCAGAGTGAAAAYAYGCACAGCGYCCAG | |
| CsL3:39 | 1882 | TTGCAATACGGCACCCAGAGTGAAAAYAYGCACAGCGYCCAC | |
| CsL3:40 | 1883 | TTGCAATACGGCACCCAGAGTGAAAAYAYGCACAGCGYCCAG | |
| CsL3:41 | 1884 | CACCAATACGGCACCCAGAGTGAAAAYAYGCACAGCTGACAC | |
| CsL3:42 | 1885 | CACCAATACGGCACCCAGAGTGAAAAYAYGCACAGCTGACAG | |
| CsL3:43 | 1886 | TTGCAATACGGCACCCAGAGTGAAAAYAYGCACAGCTGACAC | |
| CsL3:44 | 1887 | TTGCAATACGGCACCCAGAGTGAAAAYAYGCACAGCTGACAG | |
| CsL3:45 | 1888 | AGGTGTTTCCCTCTCCTCCTTAGCGACTTGGCYCTCTTGATC | 20 |
| CsL3:46 | 1889 | AGGTGTTTCCCTCTCCTCCTTAGCGACTTGCTGCTCTTGATC | |
| CsL3:47 | 1890 | TTCCCAGGCTTGTCGAAGCAGTGGCGGCATACTATCAGTAGT | 21 |
| CsL3:48 | 1891 | GCCGAACAGGAAGGCTGGCTCTGCACCTTGGTGATCTTTGAG | 22 |
| CsL3:49 | 1892 | TCTCTTCAGCTGCTTCTCCAATCCGGCTATGATCAATTCAAG | 23 |
| CsL3:50 | 1893 | CACAGGCGCGCCTTAGTGATGGTGGTGATGGTGAGACCCACTTTC | 24 |

TABLE 20
Performance of the top 20 CsL3 hits and associated residue substitutions
relative to the parent clone L8-F301.

| | Colony Assay | | | Residue and | | | | | | | | | |
|---|---|---|---|---|---|---|---|---|---|---|---|---|---|
| | Results | | | Sequence Position | | | | | | | | | |
| CsL3 Hit | REP | IND | F. IND | 60 | 64 | 82 | 86 | 100 | 113 | 121 | 126 | 128 | 134 |
| L8-F301 | 0.7 | 1.6 | 2.5 | M | Q | N | C | C | A | T | S | E | V |
| 1C12 | 0.9 | 8.8 | 9.5 | H | G | F | M | S | — | — | — | — | T |
| 1B11 | 1.3 | 10.8 | 8.0 | — | — | F | M | A | — | — | — | — | T |
| 1A07 | 1.5 | 8.1 | 5.4 | — | — | F | M | S | — | — | P | — | T |
| 1B04 | 2.2 | 10.5 | 4.8 | H | — | F | M | S | — | — | — | — | T |
| 2E09 | 1.3 | 5.7 | 4.5 | H | — | F | M | A | G | — | — | — | T |
| 2D11 | 0.9 | 3.9 | 4.3 | N | — | F | M | S | — | — | — | — | T |
| 2B09 | 0.9 | 3.8 | 4.3 | — | — | Y | M | S | — | — | — | — | T |
| 2B06 | 1.3 | 5.6 | 4.2 | H | — | F | M | S | G | — | — | — | — |
| 2A01 | 1.4 | 5.9 | 4.2 | — | G | F | M | S | — | — | — | — | T |
| 2D10 | 1.1 | 4.7 | 4.2 | H | — | F | M | S | — | — | — | — | T |
| 2D02 | 1.6 | 6.3 | 3.9 | — | — | F | M | S | — | — | P | — | T |
| 2E07 | 0.9 | 3.4 | 3.8 | — | — | Y | M | A | — | — | — | — | T |
| 2E12 | 1.2 | 4.4 | 3.8 | — | — | Y | M | A | — | — | — | — | T |
| 1C01 | 1.5 | 5.5 | 3.7 | — | G | Y | M | A | — | — | — | — | T |
| 1B05 | 1.3 | 4.8 | 3.6 | — | — | Y | M | A | — | — | — | — | T |
| 2E10 | 0.4 | 1.3 | 3.5 | H | R | Y | M | S | — | — | — | — | T |
| 2B12 | 1.7 | 6.1 | 3.5 | — | — | F | M | S | — | — | — | — | T |
| 2E08 | 2.2 | 7.6 | 3.4 | — | — | F | M | A | — | — | — | — | T |
| 2E11 | 2.1 | 7.2 | 3.4 | — | — | F | M | S | — | I | — | Q | L |
| 2D12 | 2.1 | 7.0 | 3.4 | — | S | F | M | A | G | — | — | — | T |

| | Residue and | | | | | | | | | | | |
|---|---|---|---|---|---|---|---|---|---|---|---|---|
| | Sequence Position | | | | | | | | | | | |
| CsL3 Hit | 135 | 139 | 147 | 151 | 152 | 155 | 156 | 157 | 158 | 163 | 192 | 202 |
| L8-F301 | S | V | F | S | Q | K | E | E | R | T | L | K |
| 1C12 | — | — | L | Q | — | — | — | — | — | — | — | * |
| 1B11 | — | — | L | Q | — | — | — | — | — | — | — | — |
| 1A07 | D | — | Q | G | — | — | — | — | — | — | — | — |
| 1B04 | — | — | Q | Q | — | — | — | — | — | P | — | — |
| 2E09 | — | — | Q | G | — | — | — | — | — | — | — | — |
| 2D11 | — | — | Q | — | — | — | — | — | — | — | — | — |
| 2B09 | — | — | Q | G | — | N | — | — | — | — | — | — |
| 2B06 | D | — | Q | G | — | — | — | D | — | — | — | — |
| 2A01 | — | — | Q | — | — | — | — | — | — | — | — | — |
| 2D10 | — | — | Q | — | — | — | — | — | — | — | — | — |
| 2D02 | D | — | Q | G | — | — | — | — | — | — | — | — |
| 2E07 | — | — | Q | G | — | — | — | — | — | — | — | — |
| 2E12 | G | — | Q | — | H | — | V | — | — | — | — | — |
| 1C01 | G | I | Q | — | — | — | — | — | — | — | V | — |
| 1B05 | — | — | Q | G | — | — | — | — | — | — | — | — |
| 2E10 | — | — | V | Q | — | — | — | — | T | — | — | — |
| 2B12 | — | — | Q | — | — | — | — | — | — | — | — | N |
| 2E08 | — | — | Q | G | — | — | — | — | — | — | — | — |
| 2E11 | — | — | Q | — | — | — | — | — | — | — | — | — |
| 2D12 | — | — | Q | — | — | — | — | — | — | — | — | — |

IND = induction with 20 ppb CS;
REP = repression in absence of inducer;
F. IND = fold induction (IND/REP)

### D. Sixth Round Chlorsulfuron Repressor Shuffling.

Creating Novel Diversity Through Random Mutagenesis.

In order to create new diversity for shuffling the top clone from CsL3 was subjected to error prone PCR mutagenesis using Mutazyme (Stratagene). The mutated PCR product encoding the CsR ligand binding domain was inserted into library expression vector pVER7334 as a Sac1 to Asc1 fragment, transformed into library strain Km3 and plated onto LB+40 ug/ml Kanamycin and 50 ug/ml Carbenecillin. Approximately 10,000 colonies were then replica plated onto M9 Xgal assay medium +/−20 ppb Cs. Putative hits were then re-arrayed and replica plated onto the same assay medium. Performance was gauged by the level of blue colony color after 24 hrs incubation on inducer (induction) and 72 hrs incubation without inducer (repression). The top hits were then subjected to liquid B-galactosidase assays for quantitative assessment (Table 21). The results reveal that modification of position D178 is important as mutation to either V or E improves activity at least two-fold. Substitutions F78Y, R88C, and S165R may also have made contributions to activity.

TABLE 21
Performance of the top CsL3-MTZ hits and associated residue
substitutions relative to the parent clone CsL3-C12 and L8-F301.

| | B-galacto- | | | Residue and | | | | | | | | | | | | |
|---|---|---|---|---|---|---|---|---|---|---|---|---|---|---|---|---|
| | sidase assay | | | Sequence Position | | | | | | | | | | | | |
| Clone | IND | REP | F. IND | 60 | 64 | 78 | 82 | 86 | 88 | 100 | 134 | 147 | 151 | 165 | 178 | 202 |
| L8-3F01 | 8 | 7 | 1 | M | Q | F | N | C | R | C | V | F | S | S | D | K |
| CsL3-C12 | 218 | 17 | 13 | H | G | — | F | M | — | S | T | L | Q | — | — | * |
| CsL3-C12-MTZ-2 | 287 | 9 | 30 | H | G | — | F | M | — | S | T | L | Q | — | V | * |
| CsL3-C12-MTZ-4 | 460 | 18 | 25 | H | G | Y | F | M | — | S | T | L | Q | — | E | * |
| CsL3-C12-MTZ-3 | 347 | 21 | 16 | H | G | — | F | M | C | S | T | L | Q | R | — | * |
| CsL3-C12-MTZ-5 | 440 | 29 | 15 | H | G | — | F | M | — | S | T | L | Q | — | E | * |

IND = induction with 20 ppb CS;
REP = repression in absence of inducer;
F. IND = fold induction (IND/REP)

Construction and Screening of Library CsL4.2.

Seventh round library CsL4.2 was designed based on the best diversity from CsL3 and CsL3-MTZ library screens (Table 14). The library was assembled with oligonucleotides shown below in Table 22. The first and last primers were used as rescue primers. CsL4.2 included a C-terminal 6×His-tag extension to facilitate protein purification. The library was assembled and cloned into vector pVER7334 Sac1 to Asc1, transformed into library assay strain Km3 and plated onto LB+40 ug/m1 Kanamycin and 50 ug/ml carbenecillin. Approximately 8,000 colonies were re-arrayed into 384-well format and replica plated onto M9 Xgal assay medium +/−2 ppb Cs. Putative hits were re-arrayed in 96-well format onto the same media for re-testing. Confirmed hits were then tested for induction and repression aspects in liquid culture using B-galactosidase assays. Results show that F82, L147, V178, and to a lesser extent Q151 were strongly selected for in the hit population. Although there was no preference at position 135 in the larger hit population, the top six clones all had the S135D substitution (Table 23).

TABLE 22
Library 4.2 assembly oligonucleotides.

| | SEQ | | |
|---|---|---|---|
| Oligo | ID NO | Sequence | Group |
| CsL4.2-1 | 1894 | TGGCACGTCAAGAACAAGCGAGCTCTGCTAGACGCCTTGGCC | 1 |
| CsL4.2-2 | 1895 | ATTGAGATGCATGATAGGCACGGAACCCACTACTTGCCTTTG | 2 |
| CsL4.2-3 | 1896 | ATTGAGATGCATGATAGGCACCAAACCCACTACTTGCCTTTG | |
| CsL4.2-4 | 1897 | ATTGAGATGATGGATAGGCACGGAACCCACTACTTGCCTTTG | |
| CsL4.2-5 | 1898 | ATTGAGATGATGGATAGGCACCAAACCCACTACTTGCCTTTG | |
| CsL4.2-6 | 1899 | GAAGGGGAAAGCTGGCAAGACTWTTTGAGGAACTWTGCTAAG | 3 |
| CsL4.2-7 | 1900 | AGCATGCGACKAGCTTTGCTCAGTCACCGTGATGGAGCCAAG | 4 |
| CsL4.2-8 | 1901 | AGCATGCGATGCGCTTTGCTCAGTCACCGTGATGGAGCCAAG | |
| CsL4.2-9 | 1902 | GTCKCCCTTGGTACACGGTGGACGGAGCAACAGTATGAAACT | 5 |
| CsL4.2-10 | 1903 | GCGGAGAACATGTTGGCCTTCCTGACCCAACAAGGTTTCTCC | 6 |
| CsL4.2-11 | 1904 | CTTGAGAATGCCTTGTACGCAACAGATGCTGTGCGGGTTTTC | 7 |
| CsL4.2-12 | 1905 | CTTGAGAATGCCTTGTACGCAACAAGCGCTGTGCGGGTTTTC | |
| CsL4.2-13 | 1906 | ACTCTGGGTGCCGTATTGCWGGATCAAGAGGGACAAGTCGCT | 8 |
| CsL4.2-14 | 1907 | ACTCTGGGTGCCGTATTGCWGGATCAAGAGCAACAAGTCGCT | |
| CsL4.2-15 | 1908 | AAKGAGGAGAGGGAAACACCTACTMCTGATAGWATGCCGCCA | 9 |
| CsL4.2-16 | 1909 | CTGCTTCGACAAGCCTGGGAACTCAAAGWKCACCAAGGTGCA | 10 |
| CsL4.2-17 | 1910 | GAGCCAGCCTTCCTGTTCGGCCTTGAATTGATCATAGCCGGA | 11 |
| CsL4.2-18 | 1911 | TTGGAGAAGCAGCTGAAGAGAGAAAGTGGGTCTCACCATCAC | 12 |
| CsL4.2-19 | 1912 | GTGCCTATCATGCATCTCAATGGCCAAGGCGTCTAGCAGAGC | 13 |
| CsL4.2-20 | 1913 | GTGCCTATCCATCATCTCAATGGCCAAGGCGTCTAGCAGAGC | |
| CsL4.2-21 | 1914 | GTCTTGCCAGCTTTCCCCTTCCAAAGGCAAGTAGTGGGTTCC | 14 |
| CsL4.2-22 | 1915 | GTCTTGCCAGCTTTCCCCTTCCAAAGGCAAGTAGTGGGTTTG | |
| CsL4.2-23 | 1916 | GAGCAAAGCTMGTCGCATGCTCTTAGCAWAGTTCCTCAAAWA | 15 |
| CsL4.2-24 | 1917 | GAGCAAAGCGCATCGCATGCTCTTAGCAWAGTTCCTCAAAWA | |
| CsL4.2-25 | 1918 | CCACCGTGTACCAAGGGMGACCTTGGCTCCATCACGGTGACT | 16 |
| CsL4.2-26 | 1919 | GAAGGCCAACATGTTCTCCGCAGTTTCATACTGTTGCTCCGT | 17 |
| CsL4.2-27 | 1920 | TGCGTACAAGGCATTCTCAAGGGAGAAACCTTGTTGGGTCAG | 18 |
| CsL4.2-28 | 1921 | CWGCAATACGGCACCCAGAGTGAAAACCCGCACAGCATCTGT | 19 |
| CsL4.2-29 | 1922 | CWGCAATACGGCACCCAGAGTGAAAACCCGCACAGCGCTTGT | |
| CsL4.2-30 | 1923 | AGGTGTTTCCCTCTCCTCMTTAGCGACTTGTCCCTCTTGATC | 20 |
| CsL4.2-31 | 1924 | AGGTGTTTCCCTCTCCTCMTTAGCGACTTGTTGCTCTTGATC | |
| CsL4.2-32 | 1925 | TTCCCAGGCTTGTCGAAGCAGTGGCGGCATWCTATCAGKAGT | 21 |
| CsL4.2-33 | 1926 | GCCGAACAGGAAGGCTGGCTCTGCACCTTGGTGMWCTTTGAG | 22 |
| CsL4.2-34 | 1927 | TCTCTTCAGCTGCTTCTCCAATCCGGCTATGATCAATTCAAG | 23 |
| CsL4.2-35 | 1928 | CACAGGCGCGCCTTAGTGATGGTGGTGATGGTGAGACCCACTTTC | 24 |

TABLE 23
Performance of the top 20 CsL4.2 hits and associated residue substitutions relative to
the parent clone L8-F301.

| | B-galactosidase | | | Residue and Sequence Position | | | | | | | | | | | |
|---|---|---|---|---|---|---|---|---|---|---|---|---|---|---|---|
| Clone | REP | IND | F. IND | 60 | 64 | 78 | 82 | 86 | 88 | 99 | 100 | 119 | 123 | 134 | 135 |
| L8-3F01 | 0.4 | 0.9 | 2.0 | M | Q | F | N | C | R | V | C | F | Q | V | S |
| CsL4.2-20 | 0.2 | 7.4 | 39.8 | H | — | — | F | M | L | — | S | — | — | T | D |
| CsL4.2-15 | 0.2 | 4.0 | 25.5 | H | — | — | F | M | — | — | S | — | — | T | D |
| CsL4.2-22 | 0.3 | 5.4 | 20.8 | H | — | — | F | M | — | I | A | — | — | T | D |
| CsL4.2-07 | 0.3 | 6.5 | 18.9 | H | — | Y | F | M | C | I | A | — | — | T | D |
| CsL4.2-16 | 0.3 | 3.8 | 15.2 | — | — | Y | F | M | C | — | S | — | — | T | D |
| CsL4.2-08 | 0.7 | 10.7 | 15.0 | — | — | — | F | M | — | — | A | — | H | T | D |
| CsL4.2-24 | 0.4 | 5.4 | 14.3 | H | — | Y | F | M | C | — | A | — | — | T | — |
| CsL4.2-21 | 0.2 | 3.2 | 13.2 | — | G | — | Y | M | — | — | A | C | — | T | — |
| CsL4.2-28 | 0.5 | 5.3 | 11.3 | — | — | Y | F | M | C | — | A | — | — | T | — |
| CsL4.2-30 | 0.5 | 4.9 | 10.8 | H | — | — | F | M | — | — | A | — | — | T | — |
| CsL4.2-26 | 0.3 | 3.1 | 10.6 | H | — | Y | F | M | C | — | S | — | — | T | — |
| CsL4.2-23 | 1.0 | 10.4 | 10.5 | — | — | Y | F | M | C | — | A | — | — | T | — |
| CsL4.2-04 | 0.4 | 4.3 | 10.2 | H | — | — | F | M | C | — | A | — | — | T | D |
| CsL4.2-01 | 0.4 | 3.8 | 9.8 | H | — | Y | F | M | — | — | A | — | — | T | D |
| CsL4.2-17 | 0.3 | 3.1 | 9.7 | — | — | Y | F | M | C | — | A | — | — | T | — |
| CsL4.2-12 | 0.7 | 6.4 | 9.5 | H | G | — | F | M | — | — | A | — | — | T | — |
| CsL4.2-18 | 0.7 | 6.8 | 9.3 | — | — | — | F | M | C | — | A | — | — | T | — |
| CsL4.2-27 | 0.4 | 3.2 | 9.1 | — | — | — | F | M | C | — | S | — | — | T | D |
| CsL4.2-11 | 0.5 | 4.8 | 8.9 | H | G | Y | F | M | — | — | S | — | — | T | — |

| | Residue and Sequence Position | | | | | | | | | | | |
|---|---|---|---|---|---|---|---|---|---|---|---|---|
| Clone | 147 | 151 | 155 | 156 | 157 | 163 | 165 | 171 | 178 | 193 | 202 | 204 |
| L8-3F01 | F | S | K | E | E | T | S | R | D | I | K | E |
| CsL4.2-20 | L | Q | — | — | — | P | — | — | V | — | — | — |
| CsL4.2-15 | L | Q | — | — | — | — | — | — | V | — | — | — |
| CsL4.2-22 | L | Q | — | — | — | P | — | — | — | — | — | — |
| CsL4.2-07 | L | Q | — | — | — | P | — | — | V | — | — | — |
| CsL4.2-16 | L | G | N | — | — | P | R | — | V | — | — | — |
| CsL4.2-08 | L | Q | — | — | — | P | — | — | V | — | — | — |
| CsL4.2-24 | L | G | N | — | — | P | — | — | V | — | — | — |
| CsL4.2-21 | L | Q | N | — | — | — | — | — | V | — | — | — |
| CsL4.2-28 | L | Q | — | Q | — | P | — | — | V | — | — | — |
| CsL4.2-30 | L | G | N | — | — | P | — | — | V | — | — | — |
| CsL4.2-26 | L | Q | — | — | — | P | R | — | V | — | — | — |
| CsL4.2-23 | L | Q | — | — | — | P | R | — | V | — | — | — |
| CsL4.2-04 | L | Q | N | — | G | — | — | — | E | — | — | — |
| CsL4.2-01 | L | G | — | — | — | — | — | — | V | — | — | — |
| CsL4.2-17 | L | Q | — | — | — | — | — | — | V | — | — | — |
| CsL4.2-12 | L | G | N | — | — | — | — | — | V | — | — | — |
| CsL4.2-18 | L | Q | — | — | — | P | R | — | V | L | — | — |
| CsL4.2-27 | L | Q | — | — | — | P | R | Q | E | — | — | D |
| CsL4.2-11 | L | Q | — | — | — | — | — | — | E | — | X | — |

IND = induction with 20 ppb CS;
REP = repression in absence of inducer;
F. IND = fold induction (IND/REP)

### E. In Vitro Mutagenesis of Residue D178.

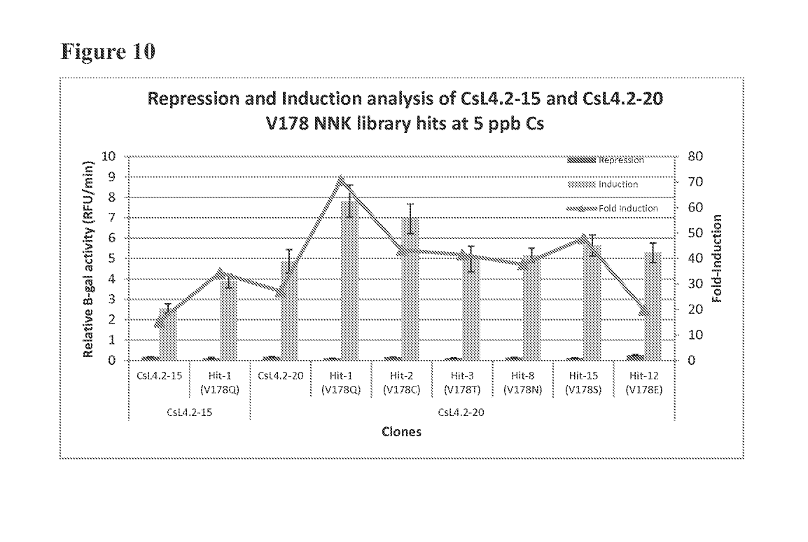

Since residue position D178 [relative to TetR(B)] was found by random mutagenesis to be important for activity further mining was sought. To this end, saturation mutagenesis was performed at this position on top CsR hits CsL4.2-15 and CsL4.2-20 using the following top and bottom strand primers in a Phusion DNA polymerase PCR reaction (New England Biolabs): GCCTGGGAACTCAAANNKCACCAAGGTGCAGAGC and GCTCTGCACCTTGGTGMNNTTTGAGTTCCCAGGC. Mutagenesis reactions were transformed into E. coli assay strain Km3 and plated onto LB+50 ug/ml Carbenecillin. Colonies were then re-arrayed into 384 well format and replica plated onto M9 Xgal assay medium +/−5 ppb Chlorsulfuron. Putative hits were then re-arrayed and analyzed by B-galactosidase assays relative to the parent clones (FIG. 10). The results show that V178 substitutions in CsL4.2-20 to C, N, Q, S, or T all yield improved activity. However, the most active substitution, V178Q, led to an approximately 2× improvement in both CsL4.2-15 and CsL4.2-20 backbones.

### F. Enhancement of Ligand Selectivity Thru Structure Guided Mutagenesis.

Chlorsulfuron (Cs) repressor CsL4.2-20 is approximately 2- and 30-fold more sensitive to Cs than Metsulfuron (Ms) and Ethametsulfuron (Es), respectively (Table 26). In order to develop non-overlapping SU herbicide responsive repressors it is desired to further separate their ligand spectrum. From the CsL4.2-20 structural model we determined that residues A56, T103, Y110, L117, L131, T134, R138, P161, M166, and A173 could potentially influence docking of related sulfonylurea compounds (e.g. note L131 and T134 in FIG. 14). Cs and Es differ in decoration of both the phenyl and triazine ring structures (circled in FIG. 14). Cs has a chloride (C1) group in the ortho position on the phenyl ring whereas it is a carboxymethyl group in Es. In addition, the meta-positions of the triazine moiety on both molecules have different substitutions: methyl and methyl-ether on Cs vs secondary amine and ethyl-ether groups on Es. Metsulfuron is essentially a hybrid between these two herbicides in that it has the triazine moiety from Cs and the phenyl moiety from Es. Saturation mutagenesis primers for each residue target are shown below. Mutagenesis reactions were carried out using Phusion DNA polymerase (New England Biolabs) and the primers listed in Table 24 and Table 25. Reactions were transformed into E. coli assay strain Km3 and plated onto LB+50 ug/ml Carbenecillin. Colonies were re-arrayed into 384-well format and replica plated onto M9 X-gal assay medium with no inducer, 10 ppb Es, 200 ppb Es, and 25 ppb Ms. Mutants having shifted selectivity relative to parent Cs activity were re-arrayed into 96-well format for further study. Putative hits were tested for repression and induction with 1, 2.5, 5, and 10 ppb Cs; 25, 50, 100, and 200 ppb Ms; and 200, 250, 300, 350, 400, 450 and 500 ppb Es. The dose of each ligand required to elicit an equal response was then used to determine relative selectivity for each clone. The ratio of Cs to Es and Cs to Ms activities as well as the relative Cs activity for the top hits is presented in Table 25. These data show that positions L131 and T134 were especially useful in modifying ligand selectivity. Mutations L131K and T134W effectively blocked Es activation: 500 ppb Es gave a similar response to 1 ppb Cs. The latter substitution unfortunately reduces Cs activity by ˜2-fold. Other residue substitutions at these positions also impact selectivity to a lesser degree. Interestingly, some mutations increased the response to Cs such as L131C while reducing, but not eliminating, Es activity. Changes in selectivity towards Ms, while occurring in most of the L131 and T134 mutants, were more modest as Cs and Ms are more similar than Cs and Es in structure.

TABLE 24
Oligonucleotides used for saturation
mutagenesis of residues potentially
involved in selectivity of different
sulfonylurea herbicides.

| Oligo | Sequence | SEQ ID NO |
|---|---|---|
| A56NNKT | GCTCTGCTAGACGCCTTGNNKATTGAGATGCA | 1929 |
| | TGATAGGC | |
| A56NNKB | GCCTATCATGCATCTCAATMNNCAAGGCGTCT | 1930 |
| | AGCAGAGC | |
| T103NNKT | GCCAAGGTCTCCCTTGGTNNKCGGTGGACGGA | 1931 |
| | GCAAC | |
| T103NNKB | GTTGCTCCGTCCACCGMNNACCAAGGGAGACC | 1932 |
| | TTGGC | |
| Y110NNKT | GGTGGACGGAGCAACAGNNKGAAACTGCGGAG | 1933 |
| | AAC | |
| Y110NNKB | GTTCTCCGCAGTTTCMNNCTGTTGCTCCGTCC | 1934 |
| | ACC | |
| L117NNKT | GAAACTGCGGAGAACATGNNKGCCTTCCTGAC | 1935 |
| | CCAAC | |
| L117NNKB | GTTGGGTCAGGAAGGCMNNCATGTTCTCCGCA | 1936 |
| | GTTTC | |
| L131NNKT | GGTTTCTCCCTTGAGAATGCCNNKTACGCAAC | 1937 |
| | AGATGC | |

TABLE 25
Oligonucleotides used for saturation
mutagenesis of residues potentially
involved in selectivity of different
sulfonylurea herbicides.

| Oligo | Sequence | SEQ ID NO |
|---|---|---|
| L131NNKB | GCATCTGTTGCGTAMNNGGCATTCTCAAGGGA | 1938 |
| | GAAACC | |
| T134NNKT | GAATGCCTTGTACGCANNKGATGCTGTGCGGG | 1939 |
| | TTTTC | |
| T134NNKB | GAAAACCCGCACAGCATCMNNTGCGTACAAGG | 1940 |
| | CATTC | |
| R138NNKT | GCAACAGATGCTGTGNNKGTTTTCACTCTGGG | 1941 |
| | TGC | |
| R138NNKB | GCACCCAGAGTGAAAACMNNCACAGCATCTGT | 1942 |
| | TGC | |
| P161NNKT | GAGGAGAGGGAAACANNKACTCCTGATAGTAT | 1943 |
| | GC | |
| P161NNKB | GCATACTATCAGGAGTMNNTGTTTCCCTCTCC | 1944 |
| | TC | |
| M166NNKT | GAAACACCTACTCCTGATAGTNNKCCGCCACT | 1945 |
| | GCTTC | |
| M166NNKB | GAAGCAGTGGCGGMNNACTATCAGGAGTAGGT | 1946 |
| | GTTTC | |
| A173NNKT | GCCACTGCTTCGACAANNKTGGGAACTCAAAG | 1947 |
| | TTC | |
| A173NNKB | GAACTTTGAGTTCCCAMNNTTGTCGAAGCAGT | 1948 |
| | GGC | |

TABLE 26
Relative Cs, Es, and Ms selectivity of various
hits based on B-galactosidase assays.

| Residue | Relative B-galactosidase activity | | |
|---|---|---|---|
| Substitution | Cs | Cs/Es | Cs/Ms |
| None | 1.0 | 30 | 2.0 |
| L131K | 1.0 | 200 | 20.0 |
| L131H | 1.0 | 80 | 3.3 |
| L131A | 0.5 | 60 | 1.7 |
| L131C | 2.0 | 60 | 4.0 |
| T134S | 0.5 | 40 | 2.5 |
| T134W | 0.5 | 100 | 1.6 |

Relative B-galactosidase activity was determined at various doses of Cs, Es, and Ms. The amount of each inducer required to achieve the same level of activity was used to determine relative ligand selectivity.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A recombinant polynucleotide construct comprising:
(a) a polynucleotide of interest operably linked to a first repressible promoter active in a plant, wherein said first repressible promoter comprises at least one operator;
(b) a polynucleotide encoding a chemically-regulated transcriptional repressor operably linked to a promoter active in said plant; and
(c) a gene silencing construct operably linked to a second repressible promoter, wherein said gene silencing construct encodes a silencing element that decreases said chemically-regulated transcriptional repressor, wherein said second repressible promoter comprises at least one operator, and wherein said chemically-regulated transcriptional repressor can bind to each of said operators in the absence of a chemical ligand and thereby repress transcription from said first and said second repressible promoters in the absence of said chemical ligand.

2. The recombinant polynucleotide construct of claim 1, wherein
(i) said first repressible promoter operably linked to said polynucleotide of interest comprises three of said operators; and/or
(ii) said promoter operably linked to said polynucleotide encoding said chemically-regulated transcriptional repressor comprises a third repressible promoter, wherein said third repressible promoter comprises at least one operator; and/or
(iii) said second repressible promoter operably linked to said gene silencing construct comprises three of said operators.

3-4. (canceled)

5. The recombinant polynucleotide construct of claim 1, wherein said polynucleotide encoding said chemically-regulated transcriptional repressor is regulated by a sulfonylurea compound.

6. (canceled)

7. The recombinant polynucleotide construct of claim 1, wherein said polynucleotide encoding said chemically-regulated transcriptional repressor is regulated by tetracycline.

8. The recombinant polynucleotide construct of claim 1, wherein said gene silencing construct encodes a cell non-autonomous silencing element that decreases said chemically-regulated transcriptional repressor.

9. The recombinant polynucleotide construct of claim 1, wherein said silencing element comprises a siRNA, a trans-acting siRNA (TAS), a hairpin RNA or an miRNA.

10-11. (canceled)

12. A plant cell comprising
(a) a first polynucleotide construct comprising a polynucleotide of interest operably linked to a first repressible promoter active in said plant cell, wherein said first repressible promoter comprises at least one operator;
(b) a second polynucleotide construct comprising a polynucleotide encoding a chemically-regulated transcriptional repressor operably linked to a promoter active in said plant cell; and,
(c) a third polynucleotide construct comprising a gene silencing construct operably linked to a second repressible promoter comprising at least one operator,
wherein (i) said gene silencing construct encodes a cell non-autonomous silencing element that decreases the level of said chemically-regulated transcriptional repressor, (ii) said second repressible promoter comprises at least one operator regulating expression of the gene silencing construct, (iii) said chemically-regulated transcriptional repressor can bind to each of said operators in the absence of a chemical ligand and thereby repress transcription of said first and said second repressible promoters in the absence of said chemical ligand, and (iv) said plant cell is tolerant to the chemical ligand.

13. The plant cell of claim 12, wherein said first, second, and third polynucleotide constructs are contained on the same recombinant polynucleotide.

14. The plant cell of claim 12, wherein
(i) said first repressible promoter operably linked to said polynucleotide of interest comprises three of said operators; and/or
(ii) said promoter operably linked to said polynucleotide encoding said chemically-regulated transcriptional repressor comprises a third repressible promoter, wherein said third repressible promoter comprises at least one operator regulating expression of said repressor; and/or
(iii) said second repressible promoter operably linked to said gene silencing construct comprises three of said operators.

15-16. (canceled)

17. The plant cell of claim 12, wherein said chemically-regulated transcriptional repressor has a chemical ligand comprising a sulfonylurea compound.

18. (canceled)

19. The plant cell of claim 12, wherein said chemically-regulated transcriptional repressor has a chemical ligand comprising tetracycline.

20. The plant cell of claim 12, wherein said gene silencing construct encodes a cell non-autonomous silencing element that decreases said chemically-regulated transcriptional repressor.

21. The plant cell of claim 12, wherein said silencing element comprises a siRNA, a trans-acting siRNA (TAS), a hairpin RNA or an amiRNA.

22-23. (canceled)

24. A plant comprising the plant cell of claim 12.

25-26. (canceled)

27. The plant of claim 24, wherein providing the plant with an effective amount of the chemical ligand (i) increases expression of said polynucleotide of interest and said silencing construct and (ii) decreases the level of said chemically-regulated transcriptional repressor in said plant or a part thereof.

28. The plant of claim 27, wherein providing an effective amount of said chemical ligand to said plant results in spatially or temporally extended expression of said polynucleotide of interest in said plant as compared to expression in a plant having been contacted with said effective amount of said chemical ligand and lacking said gene silencing construct.

29-30. (canceled)

31. The plant of claim 27, wherein providing said chemical ligand results in the complete penetration of expression of the polynucleotide of interest in the shoot apical meristem of said plant.

32. The plant of claim 27, wherein providing said chemical ligand results in the complete penetration of expression of said polynucleotide of interest throughout the plant.

33. A transformed seed of the plant of claim 24, wherein said seed comprises said first, second, and third polynucleotide construct.

34. The transformed seed of claim 33, wherein said first, second, and third polynucleotide constructs are contained on the same recombinant polynucleotide.

35. A method to regulate expression in a plant, comprising
(a) providing a plant comprising (i) a first polynucleotide construct comprising a polynucleotide encoding a chemically-regulated transcriptional repressor operably linked to a promoter active in said plant, (ii) a second polynucleotide construct comprising a polynucleotide of interest operably linked to a first repressible promoter, and (iii) a third polynucleotide construct comprising a gene silencing construct operably linked to a second repressible promoter,
wherein said gene silencing construct encodes a silencing element that decreases the level said chemically-regulated transcriptional repressor, wherein said first and second repressible promoters each comprise at least one operator, wherein said chemically-regulated transcriptional repressor can bind to each of said operators in the absence of a chemical ligand and thereby repress transcription from said first and said second repressible promoters in the absence of said chemical ligand, and wherein said plant is tolerant to said chemical ligand; and
(b) providing the plant with an effective amount of the chemical ligand whereby (i) expression of said polynucleotide of interest and said silencing construct are increased and (ii) the level of said chemically-regulated transcriptional repressor is decreased.

36-55. (canceled)
